SEQUENCE LISTING (20 sequences)

SEQ ID NO: 1 - protein, 5 aa, synthetic construct
  REGION 1..5: note = HCDR1 Sequence of anti-TRDV2 mAb for antibody B6
ENPMH

SEQ ID NO: 2 - protein, 17 aa, synthetic construct
  REGION 1..17: note = HCDR2 Sequence of anti-TRDV2 mAb for antibody B6
IIYTDTGEPTYAAEFKG

SEQ ID NO: 3 - protein, 10 aa, synthetic construct
  REGION 1..10: note = HCDR3 Sequence of anti-TRDV2 mAb for antibody B6
EGGSHWYLDV

SEQ ID NO: 4 - protein, 15 aa, synthetic construct
  REGION 1..15: note = LCDR1 Sequence of anti-TRDV2 mAb of antibody B6
RASESVDKYGISFMN

SEQ ID NO: 5 - protein, 7 aa, synthetic construct
  REGION 1..7: note = LCDR2 Sequence of anti-TRDV2 mAb of antibody B6
AASNQGS

SEQ ID NO: 6 - protein, 9 aa, synthetic construct
  REGION 1..9: note = LCDR3 Sequence of anti-TRDV2 mAb of antibody B6
QQSKEVPRT

SEQ ID NO: 7 - protein, 119 aa, synthetic construct
  REGION 1..119: note = Heavy chain sequence of anti-TRDV2 mAb of antibody B6
QIQLVQSGPELKKPGETVKISCKASGYIFTENPMHWVKQAPGKGFKWMGIIYTDTGEPTYAAEFKGRFAFSLETSASTAYLQINYIKTEDTATYFCVREGGSHWYLDVWGTGTTVTVSS

SEQ ID NO: 8 - protein, 111 aa, synthetic construct
  REGION 1..111: note = Light chain sequence of anti-TRDV2 mAb of antibody B6
DIVLTQSPASLAVSLGQRATISCRASESVDKYGISFMNWFQQKPGQPPKLLIYAASNQGSGVPARFSGSGSGTDFSLNIHPMEEDDTAMYFCQQSKEVPRTFGGGTKLEIK

SEQ ID NO: 9 - protein, 5 aa, synthetic construct
  REGION 1..5: note = HCDR1 Sequence of anti-CD33 mAb of antibody C33B904
DYAMH

SEQ ID NO: 10 - protein, 17 aa, synthetic construct
  REGION 1..17: note = HCDR2 Sequence of anti-CD33 mAb of antibody C33B904
GIGWSGGSIVYADSVKG

SEQ ID NO: 11 - protein, 10 aa, synthetic construct
  REGION 1..10: note = HCDR3 Sequence of anti-CD33 mAb of antibody C33B904
DSPYGDFFDY

SEQ ID NO: 12 - protein, 17 aa, synthetic construct
  REGION 1..17: note = LCDR1 Sequence of anti-CD33 mAb of antibody C33B904
KSSQTVFYSSNNKNYLA

SEQ ID NO: 13 - protein, 7 aa, synthetic construct
  REGION 1..7: note = LCDR2 Sequence of anti-CD33 mAb of antibody C33B904
WASTRKS

SEQ ID NO: 14 - protein, 9 aa, synthetic construct
  REGION 1..9: note = LCDR3 Sequence of anti-CD33 mAb of antibody C33B904
QHYYSTPYT

SEQ ID NO: 15 - protein, 119 aa, synthetic construct
  REGION 1..119: note = Heavy chain sequence of anti-CD33 mAb of antibody C33B904
EVQLVESGGGLVQPGRSLRLSCAASGFTFDDYAMHWVRQAPGKGLEWVSGIGWSGGSIVYADSVKGRFTISRDNAKNSLYLQMNSLRAEDTALYYCAKDSPYGDFFDYWGQGTLVTVSS

SEQ ID NO: 16 - protein, 113 aa, synthetic construct
  REGION 1..113: note = Light chain sequence of anti-CD33 mAb of antibody C33B904
DIVMTQSPDSLAVSLGERATINCKSSQTVFYSSNNKNYLAWYQQKPGQPPKLLISWASTRKSGVPDRFSGSGSGTDFTLTVSSLQAEDVAVYYCQHYYSTPYTFGQGTKLEIK

SEQ ID NO: 17 - protein, 237 aa, synthetic construct
  REGION 1..237: note = ANTI-TRDV2 (light chain) sequence expressed in CHO Cells
MAWVWTLLFLMAAAQSIQADIVLTQSPASLAVSLGQRATISCRASESVDKYGISFMNWFQQKPGQPPKLLIYAASNQGSGVPARFSGSGSGTDFSLNIHPMEEDDTAMYFCQQSKEVPRTFGGGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 18 - protein, 468 aa, synthetic construct
  REGION 1..468: note = ANTI-TRDV2 ('hole' arm) sequence expressed in CHO Cells
MAWVWTLLFLMAAAQSIQAQIQLVQSGPELKKPGETVKISCKASGYIFTENPMHWVKQAPGKGFKWMGIIYTDTGEPTYAAEFKGRFAFSLETSASTAYLQINYIKTEDTATYFCVREGGSHWYLDVWGTGTTVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLSCAVKGFYPSDIAVEWESNGQPENNYKTTPPVLDSDGSFFLVSKLTVDKSRWQQGNVFSCSVMHEALHNRFTQKSLSLSPGK

SEQ ID NO: 19 - protein, 503 aa, synthetic construct
  REGION 1..503: note = ANTI-CD33 scFv ('knob' arm) sequence expressed in CHO Cells
MAWVWTLLFLMAAAQSIQADIVMTQSPDSLAVSLGERATINCKSSQTVFYSSNNKNYLAWYQQKPGQPPKLLISWASTRKSGVPDRFSGSGSGTDFTLTVSSLQAEDVAVYYCQHYYSTPYTFGQGTKLEIKGGSEGKSSGSGSESKSTGGSEVQLVESGGGLVQPGRSLRLSCAASGFTFDDYAMHWVRQAPGKGLEWVSGIGWSGGSIVYADSVKGRFTISRDNAKNSLYLQMNSLRAEDTALYYCAKDSPYGDFFDYWGQGTLVTVSSEPKSSDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLWCLVKGFYPSDIAVEWESNGQPENNYKTTPPVLDSDGSFFLYSKLTVDKSRWQQGNVFSCSVMHEALHNHYTQKSLSLSPGK

SEQ ID NO: 20 - protein, 502 aa, synthetic construct
  REGION 1..502: note = ANTI-RSV scFv ('knob' arm) sequence expressed in CHO Cells
MAWVWTLLFLMAAAQSIQADIVMTQSPDSLAVSLGERATINCRASQSVDYNGISYMHWYQQKPGQPPKLLIYAASNPESGVPDRFSGSGSGTDFTLTISSLQAEDVAVYYCQQIIEDPWTFGQGTKVEIKGGSEGKSSGSGSESKSTGGSQITLKESGPTLVKPTQTLTLTCTFSGFSLSTSGMGVSWIRQPPGKALEWLAHIYWDDDKRYNPSLKSRLTITKDTSKNQVVLTMTNMDPVDTATYYCARLYGFTYGFAYWGQGTLVTVSSEPKSSDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLWCLVKGFYPSDIAVEWESNGQPENNYKTTPPVLDSDGSFFLYSKLTVDKSRWQQGNVFSCSVMHEALHNHYTQKSLSLSPGK

(19) United States
(12) Patent Application Publication
(10) Pub. No.: US 2026/0055185 A1
(43) Pub. Date: Feb. 26, 2026

(54) MATERIALS AND METHODS FOR MODULATING DELTA CHAIN MEDIATED IMMUNITY

(71) Applicant: JANSSEN BIOTECH, INC., HORSHAM, PA (US)

(72) Inventors: RAJKUMAR GANESAN, BLUE BELL, PA (US); IQBAL S. GREWAL, NEWTON, PA (US); SANJAYA SINGH, BLUE BELL, PA (US)

(73) Assignee: JANSSEN BIOTECH, INC., HORSHAM, PA (US)

(21) Appl. No.: 19/303,802

(22) Filed: Aug. 19, 2025

Related U.S. Application Data
(63) Continuation of application No. 17/200,287, filed on Mar. 12, 2021.
(60) Provisional application No. 62/989,111, filed on Mar. 13, 2020.

(51) Int. Cl. C07K 16/28; C07K 16/30

(52) U.S. Cl. CPC C07K 16/2809; C07K 16/2803; C07K 16/30; C07K 2317/31; C07K 2317/56; C07K 2317/565; C07K 2317/73; C07K 2317/74

(57) ABSTRACT

Anti-TRDV2 multispecific antibodies or antigen binding fragments thereof are described. Also described are nucleic acids encoding the antibodies, compositions comprising the antibodies, methods of producing the antibodies, and methods of using the antibodies for treating or preventing diseases.

### CROSS-REFERENCE TO RELATED APPLICATIONS

This application is a continuation of U.S. Ser. No. 17/200,287, filed Mar. 12, 2021, which claims the benefit of priority to U.S. Ser. No. 62/989,111 filed Mar. 13, 2020, the contents of both of which are herein incorporated by reference in their entirety.

### FIELD

This invention relates to, among other things, T Cell Receptor (TCR) redirection technologies, such as those targeting T cell Receptor Delta Variable 2 (TRDV2) molecules. Provided herein, in certain aspects, are molecules that bind TRDV2, such as monoclonal TRDV2 multispecific antibodies or epitope binding fragments thereof, including bispecific antibodies, nucleic acids and expression vectors encoding the antibodies, recombinant cells containing the vectors, and compositions comprising the antibodies. Methods of making the molecules that bind TRDV2, such as antibodies, and methods of using the antibodies to modulate an immune response to cancer cells, are also provided.

### REFERENCE TO SEQUENCE LISTING SUBMITTED ELECTRONICALLY

This application contains a sequence listing, which is submitted electronically via EFS-Web as an ASCII formatted sequence listing with a file name “14620-109-999_SL” and a creation date of Mar. 8, 2021 and having a size of 22,941 bytes. The sequence listing submitted via EFS-Web is part of the specification and is herein incorporated by reference in its entirety.

### BACKGROUND

T cells are the most abundant (˜75% of blood lymphocytes) and potent immune killer cells. The role of effector T cells in the anti-cancer immune response is strongly supported by in vitro studies and the observation that a high infiltration of CD8+ T cells in several types of cancers correlates with a favorable clinical prognostic. A few different strategies for redirecting T cells to lyse cancer cells are currently explored in clinical trials, but all have significant limitations or side effects. There is still a need in the art for improved T cell redirecting molecules and methods.

### SUMMARY

In one aspect, provided herein are molecules that bind TRDV2, such as a bispecific antibody comprising: (a) a first binding domain that binds to a TRDV2 antigen, and (b) a second binding domain that binds to an antigen on the surface of a cancer cell.

In some embodiments, the first binding domain comprises: (i) a VH comprising a VH CDR1 having an amino acid sequence of SEQ ID NO:1, a VH CDR2 having an amino acid sequence of SEQ ID NO:2, and a VH CDR3 having an amino acid sequence of SEQ ID NO:3; and (ii) a VL comprising a VL CDR1 having an amino acid sequence of SEQ ID NO:4, a VL CDR2 having an amino acid sequence of SEQ ID NO:5, and a VL CDR3 having an amino acid sequence of SEQ ID NO: 6. In some embodiments, the first binding domain comprises a VH having an amino acid sequence of SEQ ID NO:7. In some embodiments, wherein the first binding domain comprises a VL having an amino acid sequence of SEQ ID NO:8. In some embodiments, wherein the first binding domain comprises a VH having an amino acid sequence of SEQ ID NO:7, and a VL having an amino acid sequence of SEQ ID NO:8.

In some embodiments, TRDV2 is present on the surface of a γδ T cell. In some embodiments, the TRDV2 is present on the surface of a γδ T cell, and the antigen expressed on the surface of the cancer cell is a cancer antigen. In some embodiments, the cancer cell is killed when the bispecific antibody binds to the TRDV2 on the surface of the γδ T cell and the antigen on the surface of the cancer cell.

In some embodiments, the first binding domain is humanized, the second binding domain is humanized, or both the first binding domain and the second binding domain are humanized.

In some embodiments, the bispecific antibody is an IgG antibody. In some embodiments, the IgG antibody is an IgG1, IgG2, IgG3, IgG4 antibody.

In some embodiments, the bispecific antibody induces γδ T cell dependent cytotoxicity of the cancer cell in vitro with an EC₅₀ of less than about 500 pM. In some embodiments, the bispecific antibody induces γδ T cell dependent cytotoxicity of the cancer cell in vitro with an EC₅₀ of less than about 300 pM. In some embodiments, the bispecific antibody induces γδ T cell dependent cytotoxicity of the cancer cell in vitro with an EC₅₀ of less than about 160 pM. In some embodiments, the EC₅₀ is assessed with a mixture of γδ T effector cells and target cells expressing the cancer antigen. In some embodiments, the effector cell to target cell ratio is about 0.01 to 1 to about 5 to 1. In some embodiments, the effector cell to target cell ratio is about 0.1 to 1 to about 2 to 1. In some embodiments, the effector cell to target cell ratio is about 1:1.

In some embodiments, the bispecific antibody is multivalent. In some embodiments, the bispecific antibody is capable of binding at least three antigens. In some embodiments, the bispecific antibody is capable of binding at least five antigens.

In another aspect, provided is a bispecific antibody comprising: a first means capable of binding TRDV2 on the surface of the γδ T cell; and a second means capable of binding a cancer antigen. In some embodiments, the cancer antigen is on the surface of a cancer cell.

In another aspect, provided is a nucleic acid encoding a bispecific antibody provided herein. In some embodiments, also provided is a vector comprising the nucleic acid. In some embodiments, also provided is a host cell comprising the vector. In some embodiments, also provided is a kit comprising the vector and packaging for the same.

In another aspect, provided is a pharmaceutical composition comprising a bispecific antibody provided herein, and a pharmaceutically acceptable carrier. In some embodiments, provided is a method of producing the pharmaceutical composition. In some embodiments, the method comprises combining the bispecific antibody with a pharmaceutically acceptable carrier to obtain the pharmaceutical composition.

In another aspect, provided is a process for making an antibody that binds to more than one target molecule, the molecule comprising: a step for performing a function of obtaining a binding domain capable of binding to TRDV2 antigen on a γδ T cell; a step for performing a function of obtaining a binding domain capable of binding to an antigen on the surface of a cancer cell; and a step for performing a function of providing an antibody capable of binding to a TRDV2 antigen on a γδ T cell and an antigen on the surface of a cancer cell. In some embodiments, the step for performing a function of obtaining a binding domain capable of binding to an antigen on the surface of a cancer cell is repeated n times and further comprising n steps for performing a function of providing a binding domain capable of binding to a TRDV2 antigen on a γδ T cell and n number of target molecules, wherein n is at least 2.

In another aspect provided is a method of directing a γδ T cell expressing TRDV2 to a cancer cell, the method comprising contacting the γδ T cell with a bispecific antibody provided herein, wherein the contacting directs the γδ T cell to the cancer cell.

In another aspect, provided herein is a method of inhibiting growth or proliferation of cancer cells expressing a cancer antigen on the cell surface, the method comprising contacting the cancer cells with a bispecific antibody provided herein, wherein contacting the cancer cells with the pharmaceutical composition inhibits growth or proliferation of the cancer cells. In some embodiments, the cancer cells are in the presence of a γδ T cell expressing TRDV2 while in contact with the bispecific antibody.

In another aspect, provided is a method for eliminating cancer cells or treating cancer in a subject, comprising administering an effective amount of a bispecific antibody provided herein to the subject. In some embodiments, the subject is a subject in need thereof. In some embodiments, the subject is a human.

In another aspect, provided is a method of activating a γδ T cell expressing TRDV2, comprising contacting the γδ T cell with a bispecific antibody provided herein. In some embodiments, the contacting results in an increase in CD69, CD25, and/or Granzyme B expression, as compared to a control γδ T cell expressing TRDV2.

In some embodiments, the antigen on the surface of the cancer cell is a tumor-specific antigen, a tumor-associated antigen, or a neoantigen.

In some embodiments, the cancer cell is a cell of an adrenal cancer, anal cancer, appendix cancer, bile duct cancer, bladder cancer, bone cancer, brain cancer, breast cancer, cervical cancer, colorectal cancer, esophageal cancer, gallbladder cancer, gestational trophoblastic, head and neck cancer, Hodgkin lymphoma, intestinal cancer, kidney cancer, leukemia, liver cancer, lung cancer, melanoma, mesothelioma, multiple myeloma, neuroendocrine tumor, non-Hodgkin lymphoma, oral cancer, ovarian cancer, pancreatic cancer, prostate cancer, sinus cancer, skin cancer, soft tissue sarcoma spinal cancer, stomach cancer, testicular cancer, throat cancer, thyroid cancer, uterine cancer endometrial cancer, vaginal cancer, or vulvar cancer.

In some embodiments, the cancer is an adrenal cancer, anal cancer, appendix cancer, bile duct cancer, bladder cancer, bone cancer, brain cancer, breast cancer, cervical cancer, colorectal cancer, esophageal cancer, gallbladder cancer, gestational trophoblastic, head and neck cancer, Hodgkin lymphoma, intestinal cancer, kidney cancer, leukemia, liver cancer, lung cancer, melanoma, mesothelioma, multiple myeloma, neuroendocrine tumor, non-Hodgkin lymphoma, oral cancer, ovarian cancer, pancreatic cancer, prostate cancer, sinus cancer, skin cancer, soft tissue sarcoma spinal cancer, stomach cancer, testicular cancer, throat cancer, thyroid cancer, uterine cancer endometrial cancer, vaginal cancer, or vulvar cancer.

In some embodiments, the adrenal cancer is an adrenocortical carcinoma (ACC), adrenal cortex cancer, pheochromocytoma, or neuroblastoma.

In some embodiments, the anal cancer is a squamous cell carcinoma, cloacogenic carcinoma, adenocarcinoma, basal cell carcinoma, or melanoma.

In some embodiments, the appendix cancer is a neuroendocrine tumor (NET), mucinous adenocarcinoma, goblet cell carcinoid, intestinal-type adenocarcinoma, or signet-ring cell adenocarcinoma.

In some embodiments, the bile duct cancer is an extrahepatic bile duct cancer, adenocarcinomas, hilar bile duct cancer, perihilar bile duct cancer, distal bile duct cancer, or intrahepatic bile duct cancer.

In some embodiments, the bladder cancer is transitional cell carcinoma (TCC), papillary carcinoma, flat carcinoma, squamous cell carcinoma, adenocarcinoma, small-cell carcinoma, or sarcoma.

In some embodiments, the bone cancer is a primary bone cancer, sarcoma, osteosarcoma, chondrosarcoma, sarcoma, fibrosarcoma, malignant fibrous histiocytoma, giant cell tumor of bone, chordoma, or metastatic bone cancer.

In some embodiments, the brain cancer is an astrocytoma, brain stem glioma, glioblastoma, meningioma, ependymoma, oligodendroglioma, mixed glioma, pituitary carcinoma, pituitary adenoma, craniopharyngioma, germ cell tumor, pineal region tumor, medulloblastoma, or primary CNS lymphoma.

In some embodiments, the breast cancer is a breast adenocarcinoma, invasive breast cancer, noninvasive breast cancer, breast sarcoma, metaplastic carcinoma, adenocystic carcinoma, phyllodes tumor, angiosarcoma, HER2-positive breast cancer, triple-negative breast cancer, or inflammatory breast cancer.

In some embodiments, the cervical cancer is a squamous cell carcinoma, or adenocarcinoma.

In some embodiments, the colorectal cancer is a colorectal adenocarcinoma, primary colorectal lymphoma, gastrointestinal stromal tumor, leiomyosarcoma, carcinoid tumor, mucinous adenocarcinoma, signet ring cell adenocarcinoma, gastrointestinal carcinoid tumor, or melanoma.

In some embodiments, the esophageal cancer is an adenocarcinoma or squamous cell carcinoma.

In some embodiments, the gall bladder cancer is an adenocarcinoma, papillary adenocarcinoma, adenosquamous carcinoma, squamous cell carcinoma, small cell carcinoma, or sarcoma.

In some embodiments, the gestational trophoblastic disease (GTD) is a hydatidiform mole, gestational trophoblastic neoplasia (GTN), choriocarcinoma, placental-site trophoblastic tumor (PSTT), or epithelioid trophoblastic tumor (ETT).

In some embodiments, the head and neck cancer is a laryngeal cancer, nasopharyngeal cancer, hypopharyngeal cancer, nasal cavity cancer, paranasal sinus cancer, salivary gland cancer, oral cancer, oropharyngeal cancer, or tonsil cancer.

In some embodiments, the Hodgkin lymphoma is a classical Hodgkin lymphoma, nodular sclerosis, mixed cellularity, lymphocyte-rich, lymphocyte-depleted, or nodular lymphocyte-predominant Hodgkin lymphoma (NLPHL).

In some embodiments, the intestinal cancer is a small intestine cancer, small bowel cancer, adenocarcinoma, sarcoma, gastrointestinal stromal tumors, carcinoid tumors, or lymphoma.

In some embodiments, the kidney cancer is a renal cell carcinoma (RCC), clear cell RCC, papillary RCC, chromophobe RCC, collecting duct RCC, unclassified RCC, transitional cell carcinoma, urothelial cancer, renal pelvis carcinoma, or renal sarcoma.

In some embodiments, the leukemia is an acute lymphocytic leukemia (ALL), acute myeloid leukemia (AML), chronic lymphocytic leukemia (CLL), chronic myeloid leukemia (CML), hairy cell leukemia (HCL), or a myelodysplastic syndrome (MDS). In a specific embodiment, the leukemia is AML.

In some embodiments, the liver cancer is a hepatocellular carcinoma (HCC), fibrolamellar HCC, cholangiocarcinoma, angiosarcoma, or liver metastasis.

In some embodiments, the lung cancer is a small cell lung cancer, small cell carcinoma, combined small cell carcinoma, non-small cell lung cancer, lung adenocarcinoma, squamous cell lung cancer, large-cell undifferentiated carcinoma, pulmonary nodule, metastatic lung cancer, adenosquamous carcinoma, large cell neuroendocrine carcinoma, salivary gland-type lung carcinoma, lung carcinoid, mesothelioma, sarcomatoid carcinoma of the lung, or malignant granular cell lung tumor.

In some embodiments, the melanoma is a superficial spreading melanoma, nodular melanoma, acral-lentiginous melanoma, lentigo maligna melanoma, amelanotic melanoma, desmoplastic melanoma, ocular melanoma, or metastatic melanoma.

In some embodiments, the mesothelioma is a pleural mesothelioma, peritoneal mesothelioma, pericardial mesothelioma, or testicular mesothelioma.

In some embodiments, the multiple myeloma is an active myeloma or smoldering myeloma.

In some embodiments, the neuroendocrine tumor, is a gastrointestinal neuroendocrine tumor, pancreatic neuroendocrine tumor, or lung neuroendocrine tumor.

In some embodiments, the non-Hodgkin's lymphoma is an anaplastic large-cell lymphoma, lymphoblastic lymphoma, peripheral T cell lymphoma, follicular lymphoma, cutaneous T cell lymphoma, lymphoplasmacytic lymphoma, marginal zone B-cell lymphoma, MALT lymphoma, small-cell lymphocytic lymphoma, Burkitt lymphoma, chronic lymphocytic leukemia (CLL), small lymphocytic lymphoma (SLL), precursor T-lymphoblastic leukemia/lymphoma, acute lymphocytic leukemia (ALL), adult T cell lymphoma/leukemia (ATLL), hairy cell leukemia, B-cell lymphomas, diffuse large B-cell lymphoma (DLBCL), primary mediastinal B-cell lymphoma, primary central nervous system (CNS) lymphoma, mantle cell lymphoma (MCL), marginal zone lymphomas, mucosa-associated lymphoid tissue (MALT) lymphoma, nodal marginal zone B-cell lymphoma, splenic marginal zone B-cell lymphoma, lymphoplasmacytic lymphoma, B-cell non-Hodgkin lymphoma, T cell non-Hodgkin lymphoma, natural killer cell lymphoma, cutaneous T cell lymphoma, Alibert-Bazin syndrome, Sezary syndrome, primary cutaneous anaplastic large-cell lymphoma, peripheral T cell lymphoma, angioimmunoblastic T cell lymphoma (AITL), anaplastic large-cell lymphoma (ALCL), systemic ALCL, enteropathy-type T cell lymphoma (EATL), or hepatosplenic gamma/delta T cell lymphoma.

In some embodiments, the oral cancer is a squamous cell carcinoma, verrucous carcinoma, minor salivary gland carcinomas, lymphoma, benign oral cavity tumor, eosinophilic granuloma, fibroma, granular cell tumor, karatoacanthoma, leiomyoma, osteochondroma, lipoma, schwannoma, neurofibroma, papilloma, condyloma acuminatum, verruciform xanthoma, pyogenic granuloma, rhabdomyoma, odontogenic tumors, leukoplakia, erythroplakia, squamous cell lip cancer, basal cell lip cancer, mouth cancer, gum cancer, or tongue cancer.

In some embodiments, the ovarian cancer is a ovarian epithelial cancer, mucinous epithelial ovarian cancer, endometrioid epithelial ovarian cancer, clear cell epithelial ovarian cancer, undifferentiated epithelial ovarian cancer, ovarian low malignant potential tumors, primary peritoneal carcinoma, fallopian tube cancer, germ cell tumors, teratoma, dysgerminoma ovarian germ cell cancer, endodermal sinus tumor, sex cord-stromal tumors, sex cord-gonadal stromal tumor, ovarian stromal tumor, granulosa cell tumor, granulosa-theca tumor, Sertoli-Leydig tumor, ovarian sarcoma, ovarian carcinosarcoma, ovarian adenosarcoma, ovarian leiomyosarcoma, ovarian fibrosarcoma, Krukenberg tumor, or ovarian cyst.

In some embodiments, the pancreatic cancer is a pancreatic exocrine gland cancer, pancreatic endocrine gland cancer, or pancreatic adenocarcinoma, islet cell tumor, or neuroendocrine tumor.

In some embodiments, the prostate cancer is a prostate adenocarcinoma, prostate sarcoma, transitional cell carcinoma, small cell carcinoma, or neuroendocrine tumor.

In some embodiments, the sinus cancer is a squamous cell carcinoma, mucosa cell carcinoma, adenoid cystic cell carcinoma, acinic cell carcinoma, sinonasal undifferentiated carcinoma, nasal cavity cancer, paranasal sinus cancer, maxillary sinus cancer, ethmoid sinus cancer, or nasopharynx cancer.

In some embodiments, the skin cancer is a basal cell carcinoma, squamous cell carcinoma, melanoma, Merkel cell carcinoma, Kaposi sarcoma (KS), actinic keratosis, skin lymphoma, or keratoacanthoma.

In some embodiments, the soft tissue cancer is an angiosarcoma, dermatofibrosarcoma, epithelioid sarcoma, Ewing's sarcoma, fibrosarcoma, gastrointestinal stromal tumors (GISTs), Kaposi sarcoma, leiomyosarcoma, liposarcoma, dedifferentiated liposarcoma (DL), myxoid/round cell liposarcoma (MRCL), well-differentiated liposarcoma (WDL), malignant fibrous histiocytoma, neurofibrosarcoma, rhabdomyosarcoma (RMS), or synovial sarcoma.

In some embodiments, the spinal cancer is a spinal metastatic tumor.

In some embodiments, the stomach cancer is a stomach adenocarcinoma, stomach lymphoma, gastrointestinal stromal tumors, carcinoid tumor, gastric carcinoid tumors, Type I ECL-cell carcinoid, Type II ECL-cell carcinoid, or Type III ECL-cell carcinoid.

In some embodiments, the testicular cancer is a seminoma, non-seminoma, embryonal carcinoma, yolk sac carcinoma, choriocarcinoma, teratoma, gonadal stromal tumor, leydig cell tumor, or sertoli cell tumor.

In some embodiments, the throat cancer is a squamous cell carcinoma, adenocarcinoma, sarcoma, laryngeal cancer, pharyngeal cancer, nasopharynx cancer, oropharynx cancer, hypopharynx cancer, laryngeal cancer, laryngeal squamous cell carcinoma, laryngeal adenocarcinoma, lymphoepithelioma, spindle cell carcinoma, verrucous cancer, undifferentiated carcinoma, or lymph node cancer.

In some embodiments, the thyroid cancer is a papillary carcinoma, follicular carcinoma, Hürthle cell carcinoma, medullary thyroid carcinoma, or anaplastic carcinoma.

In some embodiments, the uterine cancer is an endometrial cancer, endometrial adenocarcinoma, endometroid carcinoma, serous adenocarcinoma, adenosquamous carcinoma, uterine carcinosarcoma, uterine sarcoma, uterine leiomyosarcoma, endometrial stromal sarcoma, or undifferentiated sarcoma.

In some embodiments, the vaginal cancer is a squamous cell carcinoma, adenocarcinoma, melanoma, or sarcoma.

In some embodiments, the vulvar cancer is a squamous cell carcinoma or adenocarcinoma.

In some embodiments, the cancer antigen is angiopoietin, BCMA, CD19, CD20, CD22, CD25 (IL2-R), CD30, CD33, CD37, CD38, CD52, CD56, CD123 (IL-3R), cMET, DLL/Notch, EGFR, EpCAM, FGF, FGF-R, GD2, HER2, Mesothelin, Nectin-4, PDGFRα, RANKL, SLAMF7, TROP2, VEGF, or VEGF-R.

In some embodiments, the cancer antigen is CEA, immature laminin receptor, TAG-72, HPV E6, HPV E7, BING-4, calcium-activated chloride channel 2, cyclin-B1, 9D7, EpCAM, EphA3, Her2/neu, telomerase, mesothelin, SAP-1, surviving, a BAGE family antigen, CAGE family antigen, GAGE family antigen, MAGE family antigen, SAGE family antigen, XAGE family antigen, NY-ESO-1/LAGE-1, PRAME, SSX-2, Melan-A, MART-1, Gp100, pmel17, tyrosinase, TRP-1, TRP-2, P. polypeptide, MC1R, prostate-specific antigen, β-catenin, BRCA1, BRCA2, CDK4, CML66, fibronectin, MART-2, p53, Ras, TGF-βRII, or MUC1.

In another aspect, provided herein are isolated TRDV2 bispecific antibodies or antigen binding fragments thereof, the isolated TRDV2 bispecific antibody or antigen binding fragment thereof comprising:
a. a first heavy chain (HC1);
b. a second heavy chain (HC2);
c. a first light chain (LC1); and
d. a second light chain (LC2),
   wherein HC1 is associated with LC1 and HC2 is associated with LC2, and wherein HC1 comprises a heavy chain complementarity determining region 1 (HCDR1), HCDR2, and HCDR3 comprising the amino acid sequences of SEQ ID NO:1, SEQ ID NO: 2, and SEQ ID NO:3, respectively, and LC1 comprises a light chain complementarity determining region 1 (LCDR1), LCDR2, and LCDR3 comprising the amino acid sequences of SEQ ID NO:4, SEQ ID NO:5, and SEQ ID NO:6, respectively, to form a binding site for a first antigen, and wherein HC2 and LC2 form a binding site for a second antigen.

In one embodiment, the isolated TRDV2 bispecific antibody or antigen binding fragment thereof comprises an HC1 comprising an amino acid sequence having at least 95% identity to an amino acid sequence of SEQ ID NO:7, and LC1 comprises an amino acid sequence having at least 95% identity to the amino acid sequence of SEQ ID NO:8.

In another embodiment, the isolated TRDV2 bispecific antibody or antigen binding fragment thereof comprises an HC1 comprising the amino acid sequence of SEQ ID NO:7, and LC1 comprises the amino acid sequence of SEQ ID NO: 8.

In another embodiment, the binding site for a first antigen binds to TRDV2 on a γδ T cell.

In another embodiment, the binding site for a second antigen binds to a cancer antigen present on the surface of a cancer cell.

In another embodiment, the bispecific antibody binds to TRDV2 present on the surface of the γδ T cell and the binding of the cancer antigen present on the surface of the cancer cell results in the killing of the cancer cell.

In another embodiment, TRDV2 bispecific antibody comprises a humanized HC1 and a humanized LC1.

In another embodiment, the HC2 and LC2 of the TRDV2 antibody bind to CD33. In certain embodiments, the HC2 and LC2 of the TRDV2 antibody bind to the C2 domain of CD33. In certain embodiments, the HC2 and LC2 of the TRDV2 antibody bind to the V domain of CD33.

In another embodiment the bispecific antibody or antigen binding fragment thereof is an IgG1, an IgG2, an IgG3, or an IgG4 isotype.

In a specific embodiment, the bispecific antibody or antigen binding fragment thereof is an IgG4 isotype.

In another embodiment, the TRDV2 bispecific antibody or antigen binding fragment thereof induces γδ T cell dependent cytotoxicity of a cancer cell in vitro with an EC₅₀ of less than about 500 pM.

In another embodiment, the TRDV2 bispecific antibody or antigen binding fragment thereof induces γδ T cell dependent cytotoxicity of a cancer cell in vitro with an EC₅₀ of less than about 300 pM.

In another embodiment, the TRDV2 bispecific antibody or antigen binding fragment thereof induces γδ T cell dependent cytotoxicity of a cancer cell in vitro with an EC₅₀ of less than about 160 pM.

In one embodiment, the EC₅₀ is assessed with a mixture of γδ T effector cells and Kasumi3 AML target cells.

In another embodiment, effector cell to target cell ratio is about 0.01 to 1 to about 5 to 1.

In yet another embodiment, the effector cell to target cell ratio is about 0.1 to 1 to about 2 to 1.

In a specific embodiment, the effector cell to target cell ratio is about 1:1.

In another embodiment, the TRDV2 bispecific antibody or antigen binding fragment thereof is multivalent.

In another embodiment, the TRDV2 bispecific antibody or antigen binding fragment thereof is capable of binding at least three antigens.

In another embodiment, the TRDV2 bispecific antibody or antigen binding fragment thereof is capable of binding at least five antigens.

Also provided are isolated γδ T cell bispecific antibodies or antigen binding fragments thereof, the isolated γδ T cell bispecific antibody or antigen binding fragment thereof comprising:
a. a HC1;
b. a HC2;
c. a LC1; and
d. a LC2,
   wherein HC1 is associated with LC1 and HC2 is associated with LC2, wherein HC1 and LC1 form a binding site for a first antigen on a γδ T cell, and wherein HC2 and LC2 form a binding site for a second antigen.

Also provided herein are bispecific antibodies comprising: a first means capable of specifically binding a T cell receptor gamma chain; and a second means capable of specifically binding a target molecule that is not a T cell receptor gamma chain.

Also provided are processes for making a molecule capable of specifically binding to more than one target molecule, the molecule comprising: a step for performing a function of obtaining an oligopeptide or polypeptide capable of binding to a T cell receptor gamma chain; a step for performing a function of obtaining an oligopeptide or polypeptide capable of binding to a target; and a step for performing a function of providing a molecule capable of specifically binding to a T cell receptor gamma chain and a target molecule.

In one embodiment, the step in the process for performing a function of obtaining an oligopeptide or polypeptide capable of binding to a target is repeated n times and further comprising n steps for performing a function of providing a molecule capable of specifically binding to a T cell receptor gamma chain and n number of target molecules, wherein n is at least 2.

In another aspect, provided herein are isolated anti-TRDV2/anti-CD33 bispecific antibodies or antigen binding fragments thereof comprising:
a. a HC1;
b. a HC2
c. a LC1; and
d. a LC2,
   wherein HC1 is associated with LC1 and HC2 is associated with LC2, and wherein HC1 comprises a HCDR1, HCDR2, and HCDR3 comprising the amino acid sequences of SEQ ID NO:1, SEQ ID NO:2, and SEQ ID NO:3, respectively, and LC1 comprises a LCDR1, LCDR2, and LCDR3 comprising the amino acid sequences of SEQ ID NO:4, SEQ ID NO:5, and SEQ ID NO:6, respectively, to form a binding site for a first antigen that specifically binds Vδ2, and wherein HC2 comprises a HCDR1, HCDR2, and HCDR3 comprising the amino acid sequences of SEQ ID NO:9, SEQ ID NO: 10, and SEQ ID NO: 11, respectively, and LC2 comprises a LCDR1, LCDR2, and LCDR3 comprising the amino acid sequences of SEQ ID NO: 12, SEQ ID NO:13, and SEQ ID NO:14, respectively, to form a binding site for a second antigen that specifically binds CD33.

In certain embodiments, a binding site is formed for a second antigen that specifically binds the C2 domain of CD33. In other embodiments, a binding site is formed for a second antigen that specifically binds the V domain of CD33.

In one embodiment, the isolated anti-TRDV2/anti-CD33 bispecific antibody or antigen binding fragment comprises an HC1 comprising an amino acid sequence having at least 95% identity to an amino acid sequence of SEQ ID NO:7, and LC1 comprises an amino acid sequence having at least 95% identity to the amino acid sequence of SEQ ID NO:8.

In another embodiment, the isolated anti-TRDV2/anti-CD33 bispecific antibody or antigen binding fragment comprises an HC1 comprising the amino acid sequence of SEQ ID NO:7, and LC1 comprises the amino acid sequence of SEQ ID NO: 8.

In another embodiment, the isolated anti-TRDV2/anti-CD33 bispecific antibody or antigen binding fragment comprises an HC2 comprising an amino acid sequence having at least 95% identity to the amino acid sequence of SEQ ID NO:15 and LC2 comprises an amino acid sequence having at least 95% identity to the amino acid sequence of SEQ ID NO:16.

In another embodiment, the isolated anti-TRDV2/anti-CD33 bispecific antibody or antigen binding fragment comprises an HC2 comprising the amino acid sequence of SEQ ID NO: 15 and LC2 comprises the amino acid sequence of SEQ ID NO: 16.

In another embodiment, the TRDV2 is on the surface of a γδ T cell.

In another embodiment, the CD33 is on the surface of a tumor cell or a CD34+ stem cell.

In another embodiment, the binding of the bispecific antibody to TRDV2 present on the surface of the γδ T cell and the binding of the CD33 on the surface of the cancer cell results in the killing of the cancer cell.

In another embodiment, the isolated anti-TRDV2/anti-CD33 bispecific antibody or antigen binding fragment thereof comprises a humanized HC1 and a humanized LC1.

In another embodiment, the isolated anti-TRDV2/anti-CD33 bispecific antibody or antigen binding fragment comprises a humanized HC2 and a humanized LC2.

In another embodiment, the isolated anti-TRDV2/anti-CD33 bispecific antibody or antigen binding fragment thereof is an IgG1, an IgG2, an IgG3, or an IgG4 isotype. In a specific embodiment, the bispecific antibody is an IgG4 isotype.

In another embodiment, the isolated anti-TRDV2/anti-CD33 bispecific antibody or antigen binding fragment thereof induces γδ T cell dependent cytotoxicity of a cancer cell in vitro with an EC₅₀ of less than about 500 pM.

In another embodiment, the isolated anti-TRDV2/anti-CD33 bispecific antibody or antigen binding fragment thereof induces γδ T cell dependent cytotoxicity of a cancer cell in vitro with an EC₅₀ of less than about 300 pM. In another embodiment, the isolated anti-TRDV2/anti-CD33 bispecific antibody or antigen binding fragment thereof induces γδ T cell dependent cytotoxicity of a cancer cell in vitro with an EC₅₀ of less than about 160 pM.

In one embodiment, the EC₅₀ is assessed with a mixture of γδ T effector cells and Kasumi3 AML target cells.

In another embodiment, the effector cell to target cell ratio is about 0.01 to 1 to about 5 to 1.

In yet another embodiment, the effector cell to target cell ratio is about 0.1 to 1 to about 2 to 1.

In a specific embodiment, the effector cell to target cell ratio is about 1:1.

Also provided are methods of making the isolated anti-TRDV2/anti-CD33 bispecific antibody or antigen binding fragment provided herein, the method comprising culturing a cell comprising a nucleic acid encoding the anti-TRDV2/anti-CD33 bispecific antibody or antigen binding fragment thereof under conditions to produce the bispecific antibody or antigen binding fragment thereof and recovering the bispecific antibody or antigen binding fragment thereof.

In another aspect, provided herein are isolated TRDV2 bispecific antibodies or antigen epitope binding fragments thereof, wherein the isolated TRDV2 bispecific antibodies or antigen epitope binding fragments thereof comprise a binding site for a first antigen and a binding site for a second antigen, wherein the binding site for the first antigen binds a TRDV2 epitope on a γδ T cell and the binding site for the second antigen binds an epitope of the second antigen on a surface of a target cell, and the binding of the TRDV2 epitope on the γδ T cell and the binding of the second antigen epitope on the target cell results in the killing of the target cell.

In one embodiment, the TRDV2 bispecific antibodies or antigen binding fragments comprise:
a. a HC1;
b. a HC2;
c. a LC1; and
d. a LC2,
   wherein HC1 is associated with LC1 and HC2 is associated with LC2, and wherein HC1 comprises a HCDR1, HCDR2, and HCDR3 comprising the amino acid sequences of SEQ ID NO:1, SEQ ID NO:2, and SEQ ID NO:3, respectively, and LC1 comprises a LCDR1, LCDR2, and LCDR3 comprising the amino acid sequences of SEQ ID NO:4, SEQ ID NO:5, and SEQ ID NO:6, respectively, to form the binding site for the first antigen, and wherein HC2 and LC2 form the binding site for the second antigen epitope.

In another embodiment, the TRDV2 bispecific antibodies or antigen binding fragments comprise an HC1 comprising an amino acid sequence having at least 95% identity to an amino acid sequence of SEQ ID NO:7, and LC1 comprises an amino acid sequence having at least 95% identity to the amino acid sequence of SEQ ID NO:8.

In another embodiment, the TRDV2 bispecific antibodies or antigen binding fragments comprise an HC1 comprising the amino acid sequence of SEQ ID NO: 7, and LC1 comprises the amino acid sequence of SEQ ID NO:8.

In another embodiment, the TRDV2 bispecific antibodies or antigen binding fragments comprise a humanized HC1 and a humanized LC.

In another embodiment, the TRDV2 bispecific antibodies or antigen binding fragments bind to a CD33 epitope.

In certain embodiments, the TRDV2 bispecific antibodies or antigen binding fragments bind to a CD33 C2 domain epitope. In other embodiments, the TRDV2 bispecific antibodies or antigen binding fragments bind to a CD33 V domain epitope.

In another embodiment, the TRDV2 bispecific antibodies or antigen binding fragments comprise and HC2 comprising an amino acid sequence having at least 95% identity to the amino acid sequence of SEQ ID NO:15 and LC2 comprises an amino acid sequence having at least 95% identity to the amino acid sequence of SEQ ID NO:16.

In another embodiment, the TRDV2 bispecific antibodies or antigen binding fragments comprise an HC2 comprising the amino acid sequence of SEQ ID NO: 15 and LC2 comprises the amino acid sequence of SEQ ID NO:16.

In another embodiment, the TRDV2 bispecific antibodies or antigen binding fragments thereof are an IgG1, an IgG2, an IgG3, or an IgG4 isotype. In a specific embodiment, the bispecific antibodies or antigen binding fragments thereof fragment thereof are an IgG4 isotype.

In another embodiment, the TRDV2 bispecific antibodies or antigen binding fragments thereof induce γδ T cell dependent cytotoxicity of a cancer cell in vitro with an EC₅₀ of less than about 500 pM.

In another embodiment, the TRDV2 bispecific antibodies or antigen binding fragments thereof induce yd T cell dependent cytotoxicity of a cancer cell in vitro with an EC₅₀ of less than about 300 pM.

In another embodiment, the TRDV2 bispecific antibodies or antigen binding fragments thereof induce γδ T cell dependent cytotoxicity of a cancer cell in vitro with an EC₅₀ of less than about 160 pM.

In one embodiment, the EC₅₀ is assessed with a mixture of γδ T effector cells and Kasumi3 AML target cells.

In another embodiment, the effector cell to target cell ratio is about 0.01 to 1 to about 5 to 1. In another embodiment, the effector cell to target cell ratio is about 0.1 to 1 to about 2 to 1. In a specific embodiment, the effector cell to target cell ratio is about 1:1.

Also provided are isolated γδ T cell bispecific antibodies or antigen binding fragments thereof, wherein the isolated γδ T cell bispecific antibody or antigen binding fragment thereof comprises a binding site for a first antigen epitope and a binding site for a second antigen epitope, wherein the binding site for the first antigen epitope binds a first antigen on a γδ T cell and the binding site for the second antigen epitope binds the second antigen epitope on a surface of a target cell, and the binding of the first antigen epitope on the γδ T cell and the binding of the second antigen epitope on the target cell results in the killing of the target cell.

In another aspect, provided herein are isolated nucleic acids encoding a TRDV2 bispecific antibody or antigen binding fragment thereof, the isolated TRDV2 bispecific antibody or antigen binding fragment thereof comprising:
a. a HC1;
b. a HC2;
c. a LC1; and
d. a LC2,
   wherein HC1 is associated with LC1 and HC2 is associated with LC2, and wherein HC1 comprises a HCDR1, HCDR2, and HCDR3 comprising the amino acid sequences of SEQ ID NO:1, SEQ ID NO:2, and SEQ ID NO:3, respectively, and LC1 comprises a LCDR1, LCDR2, and LCDR3 comprising the amino acid sequences of SEQ ID NO:4, SEQ ID NO:5, and SEQ ID NO:6, respectively, to form a binding site for a first antigen, and wherein HC2 and LC2 form a binding site for a second antigen.

In one embodiment, the isolated nucleic acid encodes a TRDV2 bispecific antibody comprising an HC1 comprising an amino acid sequence having at least 95% identity to the amino acid sequence of SEQ ID NO:7, and LC1 comprises an amino acid sequence having at least 95% identity to the amino acid sequence of SEQ ID NO: 8.

In another embodiment, the isolated nucleic acid encodes a TRDV2 bispecific antibody comprising an HC1 comprising the amino acid sequence of SEQ ID NO: 7, and LC1 comprises the amino acid sequence of SEQ ID NO:8.

In another embodiment, the isolated nucleic acid encodes a TRDV2 bispecific antibody comprising a binding site comprising a first antigen that binds to TRDV2 on a γδ T cell.

In another embodiment, the isolated nucleic acid encodes a TRDV2 bispecific antibody comprising a binding site for a second antigen that binds to a cancer antigen present on the surface of a cancer cell.

In another embodiment, the isolated nucleic acid encodes a TRDV2 bispecific antibody, wherein the binding of the bispecific antibody to TRDV2 present on the surface of the γδ T cell and the binding of the cancer antigen present on the surface of the cancer cell results in the killing of the cancer cell.

In another embodiment, the isolated nucleic acid encodes a TRDV2 bispecific antibody, wherein HC1 and LC1 are humanized.

In another embodiment, the isolated nucleic acid encodes a TRDV2 bispecific antibody, wherein HC2 and LC2 bind to CD33. In certain embodiments, the HC2 and LC2 bind to a CD33 C2 domain epitope. In certain embodiments, the HC2 and LC2 bind to a CD33 V domain epitope.

In another embodiment, the isolated nucleic acid encodes a TRDV2 bispecific antibody, wherein the bispecific antibody or antigen binding fragment thereof is an IgG1, an IgG2, an IgG3, or an IgG4 isotype.

In a specific embodiment, the bispecific antibody or antigen binding fragment thereof is an IgG4 isotype.

In another embodiment, the isolated nucleic acid encodes a TRDV2 bispecific antibody, wherein the bispecific antibody or antigen binding fragment thereof induces γδ T cell dependent cytotoxicity of a cancer cell in vitro with an EC₅₀ of less than about 500 pM.

In another embodiment, the isolated nucleic acid encodes a TRDV2 bispecific antibody, wherein the bispecific antibody or antigen binding fragment thereof induces γδ T cell dependent cytotoxicity of a cancer cell in vitro with an EC₅₀ of less than about 300 pM.

In another embodiment, the isolated nucleic acid encodes a TRDV2 bispecific antibody, wherein the bispecific antibody or antigen binding fragment thereof induces γδ T cell dependent cytotoxicity of a cancer cell in vitro with an EC₅₀ of less than about 160 pM.

In another embodiment, the isolated nucleic acid encodes a TRDV2 bispecific antibody, wherein the EC₅₀ is assessed with a mixture of γδ T effector cells and Kasumi3 AML target cells.

In another embodiment, the isolated nucleic acid encodes a TRDV2 bispecific antibody, wherein the effector cell to target cell ratio is about 0.01 to 1 to about 5 to 1. In one embodiment, the effector cell to target cell ratio is about 0.1 to 1 to about 2 to 1. In yet another embodiment, the effector cell to target cell ratio is about 1:1.

In another embodiment, the isolated nucleic acid encodes a TRDV2 bispecific antibody, wherein the bispecific antibody or antigen binding fragment thereof is multivalent.

In another embodiment, the isolated nucleic acid encodes a TRDV2 bispecific antibody, wherein the bispecific antibody or antigen binding fragment thereof is capable of binding at least three antigens.

In another embodiment, the isolated nucleic acid encodes a TRDV2 bispecific antibody, wherein the bispecific antibody or antigen binding fragment thereof is capable of binding at least five antigens.

Also provided are vectors comprising the isolated nucleic acids provided herein.

Also provided are host cells comprising the vectors provided herein.

Also provided are kits comprising the vectors provided herein and

packaging for the same.

In another aspect, provided herein are pharmaceutical compositions comprising an isolated TRDV2 bispecific antibody or antigen binding fragment thereof, the isolated TRDV2 bispecific antibody or antigen binding fragment thereof comprising:
a. a HC1;
b. a HC2;
c. a LC1; and
d. a LC2,
   wherein HC1 is associated with LC1 and HC2 is associated with LC2, and wherein HC1 comprises a HCDR1, HCDR2, and HCDR3 comprising the amino acid sequences of SEQ ID NO:1, SEQ ID NO:2, and SEQ ID NO:3, respectively, and LC1 comprises a LCDR1, LCDR2, and LCDR3 comprising the amino acid sequences of SEQ ID NO:4, SEQ ID NO:5, and SEQ ID NO:6, respectively, to form a binding site for a first antigen, and wherein HC2 and LC2 form a binding site for a second antigen, and a pharmaceutically acceptable carrier.

In one embodiment, the pharmaceutical composition comprises a bispecific antibody comprising an HC1 comprising an amino acid sequence having at least 95% identity to the amino acid sequence of SEQ ID NO:7, and an LC1 comprising an amino acid sequence having at least 95% identity to the amino acid sequence of SEQ ID NO:8.

In another embodiment, the pharmaceutical composition comprises a bispecific antibody comprising an HC1 comprising the amino acid sequence of SEQ ID NO: 7, and an LC1 comprising the amino acid sequence of SEQ ID NO:8.

In another embodiment, the pharmaceutical composition comprises a bispecific antibody comprising a binding site for a first antigen binds to TRDV2 on a γδ T cell.

In another embodiment, the pharmaceutical composition comprises a bispecific antibody, wherein the binding site for a second antigen binds to a cancer antigen present on the surface of a cancer cell.

In another embodiment, the pharmaceutical composition comprises a bispecific antibody, wherein the binding of the bispecific antibody to TRDV2 present on the surface of the yd T cell and the binding of the cancer antigen present on the surface of the cancer cell results in the killing of the cancer cell.

In another embodiment, the pharmaceutical composition comprises a bispecific antibody, wherein HC1 and LC1 are humanized.

In another embodiment, the pharmaceutical composition comprises a bispecific antibody, wherein HC2 and LC2 bind to CD33. In certain embodiments, the pharmaceutical composition comprises a bispecific antibody, wherein HC2 and LC2 bind to a CD33 C2 domain epitope. In other embodiments, the pharmaceutical composition comprises a bispecific antibody, wherein HC2 and LC2 bind to a CD33 V domain epitope.

In another embodiment, the pharmaceutical composition comprises a bispecific antibody, wherein the bispecific antibody or antigen binding fragment thereof is an IgG1, an IgG2, an IgG3, or an IgG4 isotype.

Also provided are methods of directing a Vδ2-expressing γδ T cell to a cancer cell, the method comprising contacting a Vδ2-expressing γδ T cell with the pharmaceutical compositions provided herein, wherein contacting the Vδ2-expressing yd T cell with the pharmaceutical composition directs the Vδ2-expressing γδ T cell to a cancer cell.

Also provided are methods of inhibiting growth or proliferation of cancer cells expressing a cancer antigen on the cell surface, the method comprising contacting the cancer cells with the pharmaceutical compositions provided herein, wherein contacting the cancer cells with the pharmaceutical composition inhibits growth or proliferation of the cancer cells.

In one embodiment, the cancer cell is in the presence of a Vδ2-expressing γδ T cell while in contact with anti-TRDV2 bispecific antibody or antigen binding fragment thereof.

Also provided are methods for treating a cancer in a subject in need thereof, the method comprising:
a. identifying a subject in need of cancer treatment; and
b. administering to the subject in need thereof the pharmaceutical compositions provided herein,
   wherein administering the pharmaceutical composition to the subject in need thereof treats the cancer in the subject.

Also provided are methods of activating a Vδ2-expressing γδ T cell, the method comprising contacting the Vδ2-expressing γδ T cell with a pharmaceutical composition provided herein, wherein contacting the Vδ2-expressing γδ T cell with the pharmaceutical composition results in an increase in CD69, CD25, and/or Granzyme B expression as compared to a control Vδ2-expressing γδ T cell.

Also provided are methods of producing the pharmaceutical composition provided herein, the method comprising combining the bispecific antibody or antigen binding fragment thereof with a pharmaceutically acceptable carrier to obtain the pharmaceutical composition.

### BRIEF DESCRIPTION OF THE DRAWINGS

The foregoing summary, as well as the following detailed description of specific embodiments of the present application, will be better understood when read in conjunction with the appended drawings. It should be understood, however, that the application is not limited to the precise embodiments shown in the drawings.

FIG. 1 shows a schematic demonstrating the binding of an exemplary anti-TRDV2/anti-CD33 bispecific antibody to recruit γδ T-cells to a cancer cell that is CD33+ and to induce cancer cell death.

FIG. 2 shows the integrity of the VG56 bispecific antibody when assessed by SDS-PAGE (reducing and non-reducing) gel.

FIG. 3 shows a graph demonstrating that Zoledronic acid selectively expands Vγ9Vδ2 cells from whole peripheral blood mononuclear cells (PBMCs).

FIG. 4 shows the binding of an anti-CD33 antibody (clone C33B904) to a MOLM-13 tumor cell line as measured by FACS. The EC₅₀ for MOLM-13 (high receptor density) was 134.3 nM

FIG. 5 shows the binding of an anti-CD33 antibody (clone C33B904) to a Kasumi-1 tumor cell line as measured by FACS. The EC₅₀ for Kasumi-1 (moderate density) was 82.2 nM.

FIG. 6 shows the binding of an anti-CD33 antibody (clone C33B904) to a OCI-AML-3 tumor cell line as measured by FACS. The EC₅₀ for OCI-AML-3 (low surface density) was 16.4 nM.

FIG. 7 shows Vδ2×CD33 bispecific-mediated whole PBMC-based cytotoxicity against Kasumi-3 cells with E:T ratio of 1:1. The EC₅₀ value for Vδ2×CD33 (VG56) was 92.8 pM.

FIG. 8 shows a graph demonstrating that the anti-TRDV2/anti-CD33 bispecific antibody mediated γδ T cell cytotoxicity against CD33 expressing Kasumi-3 cells at 1:1 effector to target cell ratio. The effector cells were enriched γδ T cells isolated from PBMCs.

FIG. 9 shows a graph demonstrating that the anti-TRDV2/anti-CD33 bispecific antibody mediated γδ T cell cytotoxicity against CD33 expressing Kasumi-3 cells at 1:1 effector to target cell ratio. The effector cells were enriched γδ T cells isolated from PBMCs.

### DETAILED DESCRIPTION

T cells are the most abundant (˜75% of blood lymphocytes) and potent immune killer cells. The role of effector T cells in the anti-cancer immune response is strongly supported by in vitro studies and the observation that a high infiltration of CD8+ T cells in several types of cancers correlates with a favorable clinical prognostic.

Recently, substantial progress has been made to harness the therapeutic potential of T cells for the treatment of cancers. Two different strategies to redirect T cells to lyse cancer cells are currently explored in clinical trials: 1) Donor T cells engineered ex vivo with chimeric antigen receptor (CAR) by using antibody fragments that bind to cancer cells and 2) recombinant bispecific protein therapeutics consisting of one arm binding to CD3 on T cells while the second arm binding to a cancer-associated antigen. Focusing on the latter, bispecific proteins allow efficient engagement of T cells with cancer cells. This results in CD3 co-receptor stimulation induced by cancer-bound bispecific molecules, which elicits an MHC-independent polyclonal T cell activation and potent cancer cell lysis. This approach bypasses some of the cancer specific tolerance mechanisms and allows the recruited T cells to kill the cancer cells.

Indeed, one of the CD3 bispecific proteins, blinatumomab, a CD3/CD19 Bispecific T Cell Engager (BiTE) has been approved by the FDA for the treatment of refractory B-acute lymphoblastic leukemia (ALL). Although, the mechanism of action of how BiTE like molecules work is still not fully understood, it does provide evidence that bi-specific reagents can induce formation of an artificial lytic synapse between the two cells, which mimics the naturally occurring lytic mediated killing of cancer cells by T cells. Preclinical experiments with chimeric antigen receptor (CAR) T cells and blinatumomab has validated the concept and provided strong rationale for this approach, and clinical trials have now provided proof of concept (POC) in human patients. Due to the clinical success of this approach, the field of CD3-directed bispecifics is rapidly growing, and a variety of antibody formats are being used to generate therapeutics to target a large number of cancer antigens. Some of the formats hold the promise of mitigation of key issues seen with blinatumomab, for example, blinatumomab is rapidly cleared from circulation and requires a continuous i.v. infusion during 4-week treatment cycle. New formats are designed for a longer serum half-life, thereby circumventing the continuous infusion.

Since T cell mediated responses are extremely potent, severe side effects can arise by inducing cytokine storm or directing T cells towards healthy tissues that express low levels of target antigen. Most CD3 bispecific proteins currently in clinical trials are targeting receptors where expression is confined to the hematopoietic lineage (CD19, CD20, CD123, etc.), or highly specific cancer antigens, such as CEA, PSMA, and MHCI-gp100. Thus, the applicability of CD3-based redirection could be limited to antigens with cancer specificity or hematological cancers, which impedes the application to many solid cancer types. In addition, CD3-directed T cell redirection with currently available technologies has not shown much efficacy in solid cancers due to various reasons (e.g., recruiting all types of CD3+ T cells, including immature, CD4+, Tregs, pan CD8 (no CTL), exhausted T cells, etc., that could lead to inefficient cancer removal; premature T cell activation that could result in a narrow therapeutic index; suboptimal T cell activation; T cell exhaustion or activation induced death of T cells; induction of cytokine release syndrome that could limit optimal dosing level; inhibition of cancer cell apoptosis; less activation of anti-cancer adaptive immune response; limited ability to combine with other immunotherapies, etc.).

Although, redirecting T cells via CD3 is attractive, as it results in a polyclonal cytotoxic response bypassing the classical antigen-specific T cell response, it raises two key concerns: 1) CD3+ T cells can be indiscriminately stimulated including various immunoregulatory and immunosuppressive T cells, which are described as playing an active role in immune evasion, and 2) Pan T cell activation that can result in severe side effects can arise by inducing cytokine storm. Thus, redirection via CD3 could potentially result in suboptimal efficacy and a narrow therapeutic index. To alleviate some of the CD3-redirection limitations, alternative strategies to re-direct T cells to cancer cells must be sought. One approach would be to select re-direction of only cytotoxic cells (a subset) that are capable of lysing cancer cells rather than indiscriminately stimulating and recruiting pan-T cells.

Another way to recruit T cells is to target specific subset of T cells. Recently, γδ T cells have provided a great interest in the cancer immunotherapy field. These unconventional T cells, well known for their innate immunity, represent only a minor proportion of the peripheral CD3+ T cells (1%-5%), but constitute a major subset (20%-50%) of T cells in epithelial tissues.

Circulating γδ T cells mainly express heterodimers of Vγ9 (TRGV9) and Vδ2 (TRDV2) chains whereas tissue γδ T cells preferentially express Vδ1 chains associated with different Vγ chains.

In humans, γδ T cells are endowed with potent anti-cancer functions (high cytotoxicity and interferon γ secretion). Moreover, γδ T cells are capable of phagocytosis, a function previously exclusive to innate myeloid lineage cells, and behave as efficient antigen-presenting cells for αβ T cells and induce adaptive immune response. γδ T cells have been shown to infiltrate cancers, but the clinical relevance of their presence is still debated. Up to now, all the research efforts have been focused on Vγ9Vδ2 T cells, and mainly aimed at activating γδ T cells in vivo or ex vivo for adoptive transfer. Although clinical studies are not yet abundant, preliminary data highlight the importance of considering the γδ T cells subset in T cell-based immunotherapy.

Thus, against this backdrop, approaches that help to overcome the limitations of CD3-based redirection, avoid pan activation of T cells and induce potent cancer lysis by selectively recruiting γδ T cells are sought. Specifically, strategies centered on bispecific antibody therapies with one arm binding to a cancer-associated antigen and the other arm binding to γδ T cell expressed TRDV2 receptor to recruit and activate γδ T cells, could address this unmet medical need by having a bispecific antibody that binds to an antigen on a bonafide cytotoxic T cell and an antigen expressed on a cancer cell in the treatment of cancers.

Various publications, articles and patents are cited or described in the background and throughout the specification; each of these references is herein incorporated by reference in its entirety. Discussion of documents, acts, materials, devices, articles or the like which has been included in the present specification is for the purpose of providing context for the invention. Such discussion is not an admission that any or all of these matters form part of the prior art with respect to any inventions disclosed or claimed.

Unless defined otherwise, all technical and scientific terms used herein have the same meaning as commonly understood to one of ordinary skill in the art to which this invention pertains. Otherwise, certain terms used herein have the meanings as set forth in the specification.

It must be noted that as used herein and in the appended claims, the singular forms “a,” “an,” and “the” include plural reference unless the context clearly dictates otherwise.

Unless otherwise stated, any numerical values, such as a concentration or a concentration range described herein, are to be understood as being modified in all instances by the term “about.” Thus, a numerical value typically includes ±10% of the recited value. For example, a concentration of 1 mg/mL includes 0.9 mg/mL to 1.1 mg/mL. Likewise, a concentration range of 1% to 10% (w/v) includes 0.9% (w/v) to 11% (w/v). As used herein, the use of a numerical range expressly includes all possible subranges, all individual numerical values within that range, including integers within such ranges and fractions of the values unless the context clearly indicates otherwise.

Unless otherwise indicated, the term “at least” preceding a series of elements is to be understood to refer to every element in the series. Those skilled in the art will recognize or be able to ascertain using no more than routine experimentation, many equivalents to the specific embodiments of the invention described herein. Such equivalents are intended to be encompassed by the invention.

As used herein, the terms “comprises,” “comprising,” “includes,” “including,” “has,” “having,” “contains” or “containing,” or any other variation thereof, will be understood to imply the inclusion of a stated integer or group of integers but not the exclusion of any other integer or group of integers and are intended to be non-exclusive or open-ended. For example, a composition, a mixture, a process, a method, an article, or an apparatus that comprises a list of elements is not necessarily limited to only those elements but can include other elements not expressly listed or inherent to such composition, mixture, process, method, article, or apparatus. Further, unless expressly stated to the contrary, “or” refers to an inclusive or and not to an exclusive or. For example, a condition A or B is satisfied by any one of the following: A is true (or present) and B is false (or not present), A is false (or not present) and B is true (or present), and both A and B are true (or present).

As used herein, the conjunctive term “and/or” between multiple recited elements is understood as encompassing both individual and combined options. For instance, where two elements are conjoined by “and/or,” a first option refers to the applicability of the first element without the second. A second option refers to the applicability of the second element without the first. A third option refers to the applicability of the first and second elements together. Any one of these options is understood to fall within the meaning, and therefore satisfy the requirement of the term “and/or” as used herein. Concurrent applicability of more than one of the options is also understood to fall within the meaning, and therefore satisfy the requirement of the term “and/or.”

As used herein, the term “consists of,” or variations such as “consist of” or “consisting of,” as used throughout the specification and claims, indicate the inclusion of any recited integer or group of integers, but that no additional integer or group of integers can be added to the specified method, structure, or composition.

As used herein, the term “consists essentially of,” or variations such as “consist essentially of” or “consisting essentially of,” as used throughout the specification and claims, indicate the inclusion of any recited integer or group of integers, and the optional inclusion of any recited integer or group of integers that do not materially change the basic or novel properties of the specified method, structure or composition. Sec M.P.E.P. § 2111.03.

As used herein, “subject” means any animal, such as a mammal, such as a human. The term “mammal” as used herein, encompasses any mammal. Examples of mammals include, but are not limited to, cows, horses, sheep, pigs, cats, dogs, mice, rats, rabbits, guinea pigs, monkeys, humans, etc. In a specific embodiment, the mammal is a human.

It should also be understood that the terms “about,” “approximately,” “generally,” “substantially,” and like terms, used herein when referring to a dimension or characteristic of a component of the preferred invention, indicate that the described dimension/characteristic is not a strict boundary or parameter and does not exclude minor variations therefrom that are functionally the same or similar, as would be understood by one having ordinary skill in the art. At a minimum, such references that include a numerical parameter would include variations that, using mathematical and industrial principles accepted in the art (e.g., rounding, measurement or other systematic errors, manufacturing tolerances, etc.), would not vary the least significant digit.

The terms “identical” or percent “identity,” in the context of two or more nucleic acids or polypeptide sequences (e.g., anti-TRDV2/anti-cancer-associated antigen bispecific antibodies and polynucleotides that encode them, anti-TRDV2/anti-CD33 bispecific antibodies and polynucleotides that encode them, TRDV2 polypeptides and TRDV2 polynucleotides that encode them, CD33 polypeptides and CD33 polynucleotides that encode them), refer to two or more sequences or subsequences that are the same or have a specified percentage of amino acid residues or nucleotides that are the same, when compared and aligned for maximum correspondence, as measured using one of the following sequence comparison algorithms or by visual inspection.

For sequence comparison, typically one sequence acts as a reference sequence, to which test sequences are compared. When using a sequence comparison algorithm, test and reference sequences are input into a computer, subsequence coordinates are designated, if necessary, and sequence algorithm program parameters are designated. The sequence comparison algorithm then calculates the percent sequence identity for the test sequence(s) relative to the reference sequence, based on the designated program parameters.

Optimal alignment of sequences for comparison can be conducted, e.g., by the local homology algorithm of Smith & Waterman, Adv. Appl. Math. 2:482 (1981), by the homology alignment algorithm of Needleman & Wunsch, J. Mol. Biol. 48:443 (1970), by the search for similarity method of Pearson & Lipman, Proc. Nat'l. Acad. Sci. USA 85:2444 (1988), by computerized implementations of these algorithms (GAP, BESTFIT, FASTA, and TFASTA in the Wisconsin Genetics Software Package, Genetics Computer Group, 575 Science Dr., Madison, WI), or by visual inspection (see generally, Current Protocols in Molecular Biology, F. M. Ausubel et al., eds., Current Protocols, a joint venture between Greene Publishing Associates, Inc. and John Wiley & Sons, Inc., (1995 Supplement) (Ausubel)).

Examples of algorithms that are suitable for determining percent sequence identity and sequence similarity are the BLAST and BLAST 2.0 algorithms, which are described in Altschul et al. (1990) J. Mol. Biol. 215:403-410 and Altschul et al. (1997) Nucleic Acids Res. 25:3389-3402, respectively. Software for performing BLAST analyses is publicly available through the National Center for Biotechnology Information. This algorithm involves first identifying high scoring sequence pairs (HSPs) by identifying short words of length W in the query sequence, which either match or satisfy some positive-valued threshold score T when aligned with a word of the same length in a database sequence. T is referred to as the neighborhood word score threshold (Altschul et al., supra). These initial neighborhood word hits act as seeds for initiating searches to find longer HSPs containing them. The word hits are then extended in both directions along each sequence for as far as the cumulative alignment score can be increased.

Cumulative scores are calculated using, for nucleotide sequences, the parameters M (reward score for a pair of matching residues; always >0) and N (penalty score for mismatching residues; always <0). For amino acid sequences, a scoring matrix is used to calculate the cumulative score. Extension of the word hits in each direction are halted when: the cumulative alignment score falls off by the quantity X from its maximum achieved value; the cumulative score goes to zero or below, due to the accumulation of one or more negative-scoring residue alignments; or the end of either sequence is reached. The BLAST algorithm parameters W, T, and X determine the sensitivity and speed of the alignment. The BLASTN program (for nucleotide sequences) uses as defaults a word length (W) of 11, an expectation (E) of 10, M=5, N=−4, and a comparison of both strands. For amino acid sequences, the BLASTP program uses as defaults a word length (W) of 3, an expectation (E) of 10, and the BLOSUM62 scoring matrix (scc Henikoff & Henikoff, Proc. Natl. Acad. Sci. USA 89:10915 (1989)).

In addition to calculating percent sequence identity, the BLAST algorithm also performs a statistical analysis of the similarity between two sequences (see, e.g., Karlin & Altschul, Proc. Nat'l. Acad. Sci. USA 90:5873-5787 (1993)). One measure of similarity provided by the BLAST algorithm is the smallest sum probability (P(N)), which provides an indication of the probability by which a match between two nucleotide or amino acid sequences would occur by chance. For example, a nucleic acid is considered similar to a reference sequence if the smallest sum probability in a comparison of the test nucleic acid to the reference nucleic acid is less than about 0.1, such as less than about 0.01, or less than about 0.001.

A further indication that two nucleic acid sequences or polypeptides are substantially identical is that the polypeptide encoded by the first nucleic acid is immunologically cross reactive with the polypeptide encoded by the second nucleic acid, as described below. Thus, a polypeptide is typically substantially identical to a second polypeptide, for example, where the two peptides differ only by conservative substitutions. Another indication that two nucleic acid sequences are substantially identical is that the two molecules hybridize to each other under stringent conditions.

As used herein, the term “polynucleotide,” synonymously referred to as “nucleic acid molecule,” “nucleotides” or “nucleic acids,” refers to any polyribonucleotide or polydeoxyribonucleotide, which can be unmodified RNA or DNA or modified RNA or DNA. “Polynucleotides” include, without limitation single- and double-stranded DNA, DNA that is a mixture of single- and double-stranded regions, single- and double-stranded RNA, and RNA that is mixture of single- and double-stranded regions, hybrid molecules comprising DNA and RNA that can be single-stranded or, more typically, double-stranded or a mixture of single- and double-stranded regions. In addition, “polynucleotide” refers to triple-stranded regions comprising RNA or DNA or both RNA and DNA. The term polynucleotide also includes DNAs or RNAs containing one or more modified bases and DNAs or RNAs with backbones modified for stability or for other reasons. “Modified” bases include, for example, tritylated bases and unusual bases such as inosine. A variety of modifications can be made to DNA and RNA; thus, “polynucleotide” embraces chemically, enzymatically or metabolically modified forms of polynucleotides as typically found in nature, as well as the chemical forms of DNA and RNA characteristic of viruses and cells. “Polynucleotide” also embraces relatively short nucleic acid chains, often referred to as oligonucleotides.

As used herein, the term “vector” is a replicon in which another nucleic acid segment can be operably inserted so as to bring about the replication or expression of the segment.

As used herein, the term “host cell” refers to a cell comprising a nucleic acid molecule provided herein. The “host cell” can be any type of cell, e.g., a primary cell, a cell in culture, or a cell from a cell line. In one embodiment, a “host cell” is a cell transfected with a nucleic acid molecule provided herein. In another embodiment, a “host cell” is a progeny or potential progeny of such a transfected cell. A progeny of a cell may or may not be identical to the parent cell, e.g., due to mutations or environmental influences that can occur in succeeding generations or integration of the nucleic acid molecule into the host cell genome.

The term “expression” as used herein, refers to the biosynthesis of a gene product. The term encompasses the transcription of a gene into RNA. The term also encompasses translation of RNA into one or more polypeptides, and further encompasses all naturally occurring post-transcriptional and post-translational modifications. The expressed bispecific antibody can be within the cytoplasm of a host cell, into the extracellular milieu such as the growth medium of a cell culture or anchored to the cell membrane.

As used herein, the terms “peptide,” “polypeptide,” or “protein” can refer to a molecule comprised of amino acids and can be recognized as a protein by those of skill in the art. The conventional one-letter or three-letter code for amino acid residues is used herein. The terms “peptide,” “polypeptide,” and “protein” can be used interchangeably herein to refer to polymers of amino acids of any length. The polymer can be linear or branched, it can comprise modified amino acids, and it can be interrupted by non-amino acids. The terms also encompass an amino acid polymer that has been modified naturally or by intervention; for example, disulfide bond formation, glycosylation, lipidation, acetylation, phosphorylation, or any other manipulation or modification, such as conjugation with a labeling component. Also included within the definition are, for example, polypeptides containing one or more analogs of an amino acid (including, for example, unnatural amino acids, etc.), as well as other modifications known in the art.

The peptide sequences described herein are written according to the usual convention whereby the N-terminal region of the peptide is on the left and the C-terminal region is on the right. Although isomeric forms of the amino acids are known, it is the L-form of the amino acid that is represented unless otherwise expressly indicated.

### Antibodies

In certain aspects, provided herein are isolated anti-TRDV2 bispecific antibodies or antigen-binding fragments thereof, nucleic acids and expression vectors encoding the antibodies, recombinant cells containing the vectors, and compositions comprising the antibodies.

In certain embodiments, provided are isolated anti-TRDV2 bispecific antibodies or antigen-binding fragments thereof, nucleic acids and expression vectors encoding the antibodies, recombinant cells containing the vectors, and compositions comprising the bispecific antibodies. Methods of making the antibodies, and methods of using the antibodies to treat diseases are also provided. The antibodies disclosed herein possess one or more desirable functional properties, including but not limited to high-affinity binding to TRDV2 or high specificity to TRDV2. In certain embodiments, the antibodies disclosed herein possess the ability to treat or prevent a disease or disorder when administered to a subject alone or in combination with other therapies. In certain embodiments, the TRDV2 antibody comprises a TRDV2 antigen binding fragment. In some embodiments, the TRDV2 antibody consists of a TRDV2 antigen binding fragment. In other embodiments, the TRDV2 antibody is a multispecific TRDV2 antibody. In yet other embodiments, the multispecific TRDV2 antibody is a bispecific TRDV2 antibody. While TRDV2 antibodies are exemplified herein, it is understood that other molecules that bind to TRDV2 are also contemplated. Such molecules include other alternative binding agents, including equivalents of the antibodies and other antibody binding fragments provided herein. In addition, while TRDV2 bispecific antibodies are exemplified herein, it is understood that other TRDV2 multispecific antibodies are also contemplated. In certain embodiments, the TRDV2 bispecific is comprised in a TRDV2 multispecific antibody. In certain embodiments, the TRDV2 multispecific is a TRDV2 bispecific antibody.

In other aspects, provided herein are isolated anti-TRDV2 bispecific antibodies or antigen-binding fragments thereof, nucleic acids and expression vectors encoding the antibodies, recombinant cells containing the vectors, and compositions comprising the bispecific antibodies. Methods of making the antibodies, and methods of using the antibodies to treat diseases, including cancer, are also provided. The antibodies disclosed herein possess one or more desirable functional properties. In some embodiments, the bispecific antibodies provided herein have high-affinity binding to TRDV2. In some embodiments, the bispecific antibodies provided herein have high-affinity binding to a second target antigen. In some embodiments, the bispecific antibodies provided herein have high specificity to TRDV2. In some embodiments, the bispecific antibodies provided herein have high specificity to a second target antigen. In some embodiments, the bispecific antibodies provided herein have the ability to treat or prevent a disease or disorder when administered alone. In some embodiments, the bispecific antibodies provided herein have the ability to treat or prevent a disease or disorder when administered in combination with other therapies. In a specific embodiment, the multispecific antibody is a bispecific antibody. In some embodiments, the TRDV2 antibody comprises an antigen binding fragment thereof.

As used herein, the term “antibody” is used in a broad sense and includes immunoglobulin or antibody molecules including human, humanized, composite and chimeric antibodies and antibody fragments that are monoclonal or polyclonal. In general, antibodies are proteins or peptide chains that exhibit binding specificity to a specific antigen. Antibody structures are well known. Immunoglobulins can be assigned to five major classes (i.e., IgA, IgD, IgE, IgG and IgM), depending on the heavy chain constant domain amino acid sequence. IgA and IgG are further sub-classified as the isotypes IgAQ1, IgA2, IgG1, IgG2, IgG3 and IgG4. Accordingly, the antibodies provided herein can be of any of the five major classes or corresponding sub-classes. In specific embodiments, the antibodies provided herein are IgG1, IgG2, IgG3 or IgG4. Antibody light chains of vertebrate species can be assigned to one of two clearly distinct types, namely kappa and lambda, based on the amino acid sequences of their constant domains. Accordingly, the antibodies provided herein can contain a kappa or lambda light chain constant domain. According to particular embodiments, the antibodies provided herein include heavy and/or light chain constant regions from rat or human antibodies.

In addition to the heavy and light constant domains, antibodies contain an antigen-binding region that is made up of a light chain variable region (VL) and a heavy chain variable region (VH), each of which contains three domains (i.e., complementarity determining regions 1 (CDR1), CDR2 and CDR3. A “CDR” refers to one of three hypervariable regions (HCDR1, HCDR2 or HCDR3) within the non-framework region of the immunoglobulin (Ig or antibody) VH β-sheet framework, or one of three hypervariable regions (LCDR1, LCDR2 or LCDR3) within the non-framework region of the antibody VL β-sheet framework. Accordingly, CDRs are variable region sequences interspersed within the framework region sequences. CDR regions are well known to those skilled in the art and have been defined by, for example, Kabat as the regions of most hypervariability within the antibody variable (V) domains (Kabat et al., J. Biol. Chem. 252:6609-6616 (1977); Kabat, Adv. Prot. Chem. 32:1-75 (1978)). CDR region sequences also have been defined structurally by Chothia as those residues that are not part of the conserved β-sheet framework, and thus are able to adapt different conformations (Chothia and Lesk, J. Mol. Biol. 196:901-917 (1987)). Both terminologies are well recognized in the art. CDR region sequences have also been defined by AbM, Contact and IMGT. Exemplary CDR region sequences are illustrated herein, for example, in the Sequence Listing, and tables provided in the Examples below. The positions of CDRs within a canonical antibody variable region have been determined by comparison of numerous structures (Al-Lazikani et al., J. Mol. Biol. 273:927-948 (1997); Morea et al., Methods 20:267-279 (2000)). Because the number of residues within a hypervariable region varies in different antibodies, additional residues relative to the canonical positions are conventionally numbered with a, b, c and so forth next to the residue number in the canonical variable region numbering scheme (Al-Lazikani et al., supra (1997)). Such nomenclature is similarly well known to those skilled in the art.

The light chain variable region CDR1 domain is interchangeably referred to herein as LCDR1 or VL CDR1. The light chain variable region CDR2 domain is interchangeably referred to herein as LCDR2 or VL CDR2. The light chain variable region CDR3 domain is interchangeably referred to herein as LCDR3 or VL CDR3. The heavy chain variable region CDR1 domain is interchangeably referred to herein as HCDR1 or VH CDR1. The heavy chain variable region CDR2 domain is interchangeably referred to herein as HCDR2 or VH CDR2. The heavy chain variable region CDR1 domain is interchangeably referred to herein as HCDR3 or VH CDR3.

The term “hypervariable region”, such as a VH or VL, when used herein refers to the regions of an antibody variable region that are hypervariable in sequence and/or form structurally defined loops. Generally, antibodies comprise six hypervariable regions; three in the VH (HCDR1, HCDR2, HCDR3), and three in the VL (LCDR1, LCDR2, LCDR3). A number of hypervariable region delineations are in use and are encompassed herein. The “Kabat” CDRs are based on sequence variability and are the most commonly used (see, e.g., Kabat et al., Sequences of Proteins of Immunological Interest, 5th Ed. Public Health Service, National Institutes of Health, Bethesda, MD. (1991)). “Chothia” refers instead to the location of the structural loops (scc, e.g., Chothia and Lesk, J. Mol. Biol. 196:901-917 (1987)). The end of the Chothia CDR-HCDR1 loop when numbered using the Kabat numbering convention varies between H32 and H34 depending on the length of the loop (this is because the Kabat numbering scheme places the insertions at H35A and H35B; if neither 35A nor 35B is present, the loop ends at 32; if only 35A is present, the loop ends at 33; if both 35A and 35B are present, the loop ends at 34). The “AbM” hypervariable regions represent a compromise between the Kabat CDRs and Chothia structural loops, and are used by Oxford Molecular's AbM antibody modeling software (see, e.g., Martin, in Antibody Engineering, Vol. 2, Chapter 3, Springer Verlag). “Contact” hypervariable regions are based on an analysis of the available complex crystal structures.

Recently, a universal numbering system has been developed and widely adopted, ImMunoGeneTics (IMGT) Information System® (Lafranc et al., Dev. Comp. Immunol. 27 (1): 55-77 (2003)). IMGT is an integrated information system specializing in immunoglobulins (IG), T cell receptors (TR) and major histocompatibility complex (MHC) of human and other vertebrates. Herein, the CDRs are referred to in terms of both the amino acid sequence and the location within the light or heavy chain. As the “location” of the CDRs within the structure of the immunoglobulin variable domain is conserved between species and present in structures called loops, by using numbering systems that align variable domain sequences according to structural features, CDR and framework residues and are readily identified. This information can be used in grafting and replacement of CDR residues from immunoglobulins of one species into an acceptor framework from, typically, a human antibody. An additional numbering system (AHon) has been developed by Honegger and Plückthun, J. Mol. Biol. 309:657-670 (2001). Correspondence between the numbering system, including, for example, the Kabat numbering and the IMGT unique numbering system, is well known to one skilled in the art (see, e.g., Kabat, supra; Chothia and Lesk, supra; Martin, supra; Lefranc et al., supra). An Exemplary system, shown herein, combines Kabat and Chothia.

| | Exemplary | IMGT | Kabat | AbM | Chothia | Contact |
|---|---|---|---|---|---|---|
| V_{H} CDR1 | 26-35 | 27-38 | 31-35 | 26-35 | 26-32 | 30-35 |
| V_{H} CDR2 | 50-65 | 56-65 | 50-65 | 50-58 | 53-55 | 47-58 |
| V_{H} CDR3 | 95-102 | 105-117 | 95-102 | 95-102 | 96-101 | 93-101 |
| V_{L} CDR1 | 24-34 | 27-38 | 24-34 | 24-34 | 26-32 | 30-36 |
| V_{L} CDR2 | 50-56 | 56-65 | 50-56 | 50-56 | 50-52 | 46-55 |
| V_{L} CDR3 | 89-97 | 105-117 | 89-97 | 89-97 | 91-96 | 89-96 |

Hypervariable regions may comprise “extended hypervariable regions” as follows: 24-36 or 24-34 (LCDR1), 46-56 or 50-56 (LCDR2) and 89-97 or 89-96 (LCDR3) in the VL and 26-35 or 26-35A (HCDR1), 50-65 or 49-65 (HCDR2) and 93-102, 94-102, or 95-102 (HCDR3) in the VH. CDR sequences, reflecting each of the above numbering schemes, are provided herein, including in the Sequence Listing.

The term “constant region” or “constant domain” refers to a carboxy terminal portion of the light and heavy chain which is not directly involved in binding of the antibody to antigen but exhibits various effector function, such as interaction with the Fc receptor. The terms refer to the portion of an immunoglobulin molecule having a more conserved amino acid sequence relative to the other portion of the immunoglobulin, the variable region, which contains the antigen binding site. The constant region may contain the CH1, CH2 and CH3 regions of the heavy chain and the CL region of the light chain.

The term “framework” or “FR” residues are those variable region residues flanking the CDRs. FR residues are present, for example, in chimeric, humanized, human, domain antibodies, diabodies, linear antibodies, and bispecific antibodies. FR residues are those variable domain residues other than the hypervariable region residues or CDR residues.

As used herein, the term an “isolated antibody” refers to an antibody which is substantially free of other antibodies having different antigenic specificities (e.g., an isolated antibody that specifically binds to TRDV2 is substantially free of antibodies that do not bind to Vδ2; an isolated antibody that specifically binds to a second target (e.g., CD33) is substantially free of antibodies that do not bind to the second target (e.g., CD33). In addition, an isolated antibody is substantially free of other cellular material and/or chemicals.

As used herein, the term “monoclonal antibody” refers to an antibody obtained from a population of substantially homogeneous antibodies, i.e., the individual antibodies comprising the population are identical except for possible naturally occurring mutations that can be present in minor amounts. The monoclonal antibodies provided herein can be made by the hybridoma method, phage display technology, single lymphocyte gene cloning technology, or by recombinant DNA methods. For example, the monoclonal antibodies can be produced by a hybridoma which includes a B cell obtained from a transgenic nonhuman animal, such as a transgenic mouse or rat, having a genome comprising a human heavy chain transgene and a light chain transgene.

As used herein, the term “antigen-binding fragment” refers to an antibody fragment such as, for example, a diabody, a Fab, a Fab′, a F(ab′)2, an Fv fragment, a disulfide stabilized Fv fragment (dsFv), a (dsFv)₂, a bispecific dsFv (dsFv-dsFv′), a disulfide stabilized diabody (ds diabody), a single-chain antibody molecule (scFv), a single domain antibody (sdAb) an scFv dimer (bivalent diabody), a multispecific antibody formed from a portion of an antibody comprising one or more CDRs, a camelized single domain antibody, a nanobody, a domain antibody, a bivalent domain antibody, or any other antibody fragment that binds to an antigen but does not comprise a complete antibody structure. An antigen-binding fragment is capable of binding to the same antigen to which the parent antibody or a parent antibody fragment binds. According to particular embodiments, the antigen-binding fragment comprises a light chain variable region, a light chain constant region, and an Fd segment of the heavy chain. According to other particular embodiments, the antigen-binding fragment comprises Fab and F(ab′).

As used herein, the term “single-chain antibody” refers to a conventional single-chain antibody in the field, which comprises a heavy chain variable region and a light chain variable region connected by a short peptide of about 15 to about 20 amino acids. As used herein, the term “single domain antibody” refers to a conventional single domain antibody in the field, which comprises a heavy chain variable region and a heavy chain constant region or which comprises only a heavy chain variable region.

As used herein, the term “human antibody” refers to an antibody produced by a human or an antibody having an amino acid sequence corresponding to an antibody produced by a human made using any technique known in the art. This definition of a human antibody includes intact or full-length antibodies, fragments thereof, and/or antibodies comprising at least one human heavy and/or light chain polypeptide.

As used herein, the term “humanized antibody” refers to a non-human antibody that is modified to increase the sequence homology to that of a human antibody, such that the antigen-binding properties of the antibody are retained, but its antigenicity in the human body is reduced.

As used herein, the term “chimeric antibody” refers to an antibody wherein the amino acid sequence of the immunoglobulin molecule is derived from two or more species. The variable region of both the light and heavy chains often corresponds to the variable region of an antibody derived from one species of mammal (e.g., mouse, rat, rabbit, etc.) having the desired specificity, affinity, and capability, while the constant regions correspond to the sequences of an antibody derived from another species of mammal (e.g., human) to avoid eliciting an immune response in that species.

As used herein, the term “multispecific antibody” refers to an antibody that comprises a plurality of immunoglobulin variable domain sequences, wherein a first immunoglobulin variable domain sequence of the plurality has binding specificity for a first epitope and a second immunoglobulin variable domain sequence of the plurality has binding specificity for a second epitope. In an embodiment, the first and second epitopes do not overlap or do not substantially overlap. In an embodiment, the first and second epitopes are on different antigens, e.g., the different proteins (or different subunits of a multimeric protein). In an embodiment, a multispecific antibody comprises a third, fourth, or fifth immunoglobulin variable domain. In an embodiment, a multispecific antibody is a bispecific antibody molecule, a trispecific antibody molecule, or a tetraspecific antibody molecule.

As used herein, the term “bispecific antibody” refers to a multispecific antibody that binds no more than two epitopes or two antigens. A bispecific antibody is characterized by a first immunoglobulin variable domain sequence which has binding specificity for a first epitope (e.g., an epitope on a TRDV2 antigen) and a second immunoglobulin variable domain sequence that has binding specificity for a second epitope. In an embodiment, the first and second epitopes are on different antigens, e.g., the different proteins (or different subunits of a multimeric protein). In an embodiment, a bispecific antibody comprises a heavy chain variable domain sequence and a light chain variable domain sequence which have binding specificity for a first epitope and a heavy chain variable domain sequence and a light chain variable domain sequence which have binding specificity for a second epitope. In an embodiment, a bispecific antibody comprises a half antibody, or fragment thereof, having binding specificity for a first epitope and a half antibody, or fragment thereof, having binding specificity for a second epitope. In an embodiment, a bispecific antibody comprises a scFv, or fragment thereof, having binding specificity for a first epitope, and a scFv, or fragment thereof, having binding specificity for a second epitope. In an embodiment, the first epitope is located on TRDV2 and the second epitope is located on CD33.

The term “half antibody” as used herein refers to one immunoglobulin heavy chain associated with one immunoglobulin light chain. An exemplary half-antibody is depicted in SEQ ID NO:17. One skilled in the art will readily appreciate that a half-antibody can encompass a fragment thereof and can also have an antigen binding domain consisting of a single variable domain, e.g., originating from a camelidae.

As used herein, the term “TRDV2” refers to a polypeptide capable of forming a T cell receptor when expressed on the surface of γδ T cells. TRDV2-expressing γδ T cells are among the first T cells to develop in the human fetus and are the predominant γδ T cell subset in healthy adult peripheral blood cells. The term “TRDV2” includes any TRDV2 variant, isoform, and species homolog, which is naturally expressed by cells (including T cells) or can be expressed on cells transfected with genes or cDNA encoding the polypeptide. In specific embodiments, the TRDV2 is a human TRDV2. An exemplary human TRDV2 amino acid sequence is provided by GenBank Accession Number NG_001332.3.

The term “CD33” refers to a 67 kD single pass transmembrane glycoprotein and is a member of the sialic acid-binding immunoglobulin-like lectins (Siglecs) family. While its exact biological function is unclear, in normal individuals, it is primarily considered to be a myeloid differentiation antigen, with low expression in myeloid progenitors, neutrophils and macrophages while being highly expressed in circulating monocytes and dendritic cells. CD33 has been detected on blasts and leukemic stem cells of 85-90% of patients presenting with in acute myeloid leukemia (AML). The term “CD33” includes any CD33 variant, isoform, and species homolog, which is naturally expressed by cells or can be expressed on cells transfected with genes or cDNA encoding those polypeptides, unless noted, the “CD33” is a human CD33. A human CD33 amino acid sequence is provided by GenBank Accession Number BC028152.1.

As used herein, an antibody that “specifically binds to TRDV2” refers to an antibody that binds to a TRDV2, such as a human TRDV2, with a KD of 1×10⁻⁷ M or less, such as 1×10⁻⁸ M or less, 5×10⁻⁹ M or less, 1×10⁻⁹ M or less, 5×10⁻¹⁰ M or less, or 1×10⁻¹⁰ M or less.

As used herein, an antibody that “specifically binds to a second target antigen” refers to an antibody that binds to a second target antigen with a KD of 1×10⁻⁷ M or less, such as 1×10⁻⁸ M or less, 5×10⁻⁹ M or less, 1×10⁻⁹ M or less, 5×10⁻¹⁰ M or less, or 1×10⁻¹⁰ M or less.

As used herein, an antigen binding domain or antigen binding fragment that “specifically binds to a tumor-associated antigen” refers to an antigen binding domain or antigen binding fragment that binds a tumor-associated antigen, with a KD of 1×10⁻⁷ M or less, such as 1×10⁻⁸ M or less, 5×10⁻⁹ M or less, 1×10⁻⁹ M or less, 5×10⁻¹⁰ M or less, or 1×10⁻¹⁰ M or less.

As used herein, an antibody that “specifically binds to CD33” refers to an antibody that binds to a CD33, such as a human CD33, with a KD of 1×10⁻⁷ M or less, such as 1×10⁻⁸ M or less, 5×10⁻⁹ M or less, 1×10⁻⁹ M or less, 5×10⁻¹⁰ M or less, or 1×10⁻¹⁰ M or less. In certain embodiments, the antibody specifically binds the C2 domain of CD33. In other embodiments, the antibody specifically binds the V domain of CD33.

The term “KD” refers to the dissociation constant, which is obtained from the ratio of Kd to Ka (i.e., Kd/Ka) and is expressed as a molar concentration (M). KD values for antibodies can be determined using methods in the art in view of the present disclosure. For example, the KD of an antibody can be determined by using surface plasmon resonance, such as by using a biosensor system, e.g., a Biacore® system, or by using bio-layer interferometry technology, such as an Octet RED96 system. The smaller the value of the KD of an antibody, the higher affinity that the antibody binds to a target antigen.

In one aspect, provided herein is an antibody that binds to TRDV2. In some embodiments, the antibody comprises a heavy chain variable (VH) region and a light chain variable (VL) region. In a some embodiments, the TRDV2 antibody is not a single domain antibody or nanobody. In some embodiments, the TRDV2 antibody is a humanized antibody.

In certain embodiments, provided herein is a TRDV2 antibody comprising a VH region, VL region, VH CDR1, VH CDR2, VH CDR3, VL CDR1, VL CDR2, and/or VL CDR3 of any one of the antibodies described herein. In some embodiments, provided herein is a TRDV2 antibody comprising a VH region of any one of the antibodies described herein. In some embodiments, provided herein is a TRDV2 antibody comprising a VL region of any one of the antibodies described herein. In some embodiments, provided herein is a TRDV2 antibody comprising a VH region of any one of the antibodies described herein, and a VL region of any one of the antibodies described herein. In some embodiments, provided herein is a TRDV2 antibody comprising a VH CDR1, VH CDR2, and VH CDR3 of any one of the antibodies described herein. In some embodiments, provided herein is a TRDV2 antibody comprising a VL CDR1, VL CDR2, and VL CDR3 of any one of the antibodies described herein. In some embodiments, provided herein is a TRDV2 antibody comprising a VH CDR1, VH CDR2, and VH CDR3 of any one of the antibodies described herein; and a VL CDR1, VL CDR2, and VL CDR3 of any one of the antibodies described herein. Representative VH and VL amino acid sequences, including VH CDR1, VH CDR2, VH CDR3, VL CDR1, VL CDR2 and VL CDR3 amino acid sequences, of TRDV2 antibodies provided herein are provided in the Sequence Listing, as well as Tables 1 and 2.

In some embodiments, the TRDV2 antibody is a multispecific TRDV2 antibody provided herein. In some embodiments, the multispecific TRDV2 antibody is a bispecific TRDV2 antibody. In one embodiment, the multispecific TRDV2 antibody comprises: (a) a first binding domain that binds to TRDV2, and (b) a second binding domain that binds to a second target that is not TRDV2.

In certain embodiments, the first binding domain that binds to TRDV2 comprises a VH region, VL region, VH CDR1, VH CDR2, VH CDR3, VL CDR1, VL CDR2, and/or VL CDR3 of any one of the TRDV2 antibodies described herein. In some embodiments, the first binding domain that binds to TRDV2 comprises a VH region of any one of the TRDV2 antibodies described herein. In some embodiments, the first binding domain that binds to TRDV2 comprises a VL region of any one of the TRDV2 antibodies described herein. In some embodiments, the first binding domain that binds to TRDV2 comprises a VH region and a VL region of any one of the TRDV2 antibodies described herein. In some embodiments, the first binding domain that binds to TRDV2 comprises a VH CDR1, VH CDR2, and VH CDR3 of any one of the TRDV2 antibodies described herein. In some embodiments, the first binding domain that binds to TRDV2 comprises a VL CDR1, VL CDR2, and VL CDR3 of any one of the TRDV2 antibodies described herein. In some embodiments, the first binding domain that binds to TRDV2 comprises a VH CDR1, VH CDR2, VH CDR3, VL CDR1, VL CDR2, and VL CDR3 of any one of the TRDV2 antibodies described herein. Representative VH and VL amino acid sequences, including VH CDR1, VH CDR2, VH CDR3, VL CDR1, VL CDR2 and VL CDR3 amino acid sequences, of TRDV2 antibodies provided herein are provided in the Sequence Listing, as well as Tables 1 and 2.

In some embodiments, the second target is CD33. In some embodiments, second binding domain that binds CD33 has a VH region, VL region, VH CDR1, VH CDR2, VH CDR3, VL CDR1, VL CDR2, and/or VL CDR3 of a CD33 antibody provided herein. In some embodiments, second binding domain that binds CD33 has a VH region of a CD33 antibody provided herein. In some embodiments, second binding domain that binds CD33 has a VL region of a CD33 antibody provided herein. In some embodiments, second binding domain that binds CD33 has a VH region and a VL region of a CD33 antibody provided herein. In some embodiments, second binding domain that binds CD33 has a VH CDR1, VH CDR2, and VH CDR3 of a CD33 antibody provided herein. In some embodiments, second binding domain that binds CD33 has a VL CDR1, VL CDR2, and VL CDR3 of a CD33 antibody provided herein. In some embodiments, second binding domain that binds CD33 has a VH CDR1, VH CDR2, VH CDR3, VL CDR1, VL CDR2, and VL CDR3 of a CD33 antibody provided herein. Representative VH and VL amino acid sequences, including VH CDR1, VH CDR2, VH CDR3, VL CDR1, VL CDR2 and VL CDR3 amino acid sequences, of TRDV2 antibodies provided herein are provided in the Sequence Listing, as well as Tables 3 and 4.

In some embodiments, the antibody specifically binds TRDV2. In other embodiments, the TRDV2 is present on the surface of a T cell.

In some embodiments, the TRDV2 antibody is chimeric. In some embodiments, the TRDV2 antibody is human. In some embodiments, the TRDV2 antibody is humanized. In certain embodiments, the TRDV2 antibody is an isolated TRDV2 antibody. In certain embodiments, provided is a TRDV2 antibody that is an intact antibody.

In some embodiments, the TRDV2 antibody is an IgG antibody. In some embodiments, the TRDV2 antibody is an IgG1 antibody. In some embodiments, the TRDV2 antibody is an IgG2 antibody. In some embodiments, the TRDV2 antibody is an IgG3 antibody. In some embodiments, the TRDV2 antibody is an IgG4 antibody. In some embodiments, the TRDV2 antibody comprises a kappa light chain. In some embodiments, the TRDV2 antibody comprises a lambda light chain. In some embodiments, the TRDV2 antibody is a monoclonal antibody. In some embodiments, the TRDV2 antibody is multivalent. In some embodiments, the TRDV2 antibody is capable of binding at least three antigens. In some embodiments, the TRDV2 antibody is capable of binding at least four antigens. In some embodiments, the TRDV2 antibody is capable of binding at least five antigens. In some embodiments, the TRDV2 antibody is a multispecific antibody. In some embodiments, the TRDV2 antibody is a bispecific antibody. In some embodiments, the TRDV2 antibody is a trispecific antibody. In some embodiments, the TRDV2 antibody is a quadraspecific antibody.

In other embodiments, provided is a TRDV2 antibody is an antigen binding fragment of the TRDV2 antibody. In some embodiments, the antigen binding fragment of the TRDV2 antibody is a functional fragment. In some embodiments, the TRDV2 antigen binding fragment is chimeric. In some embodiments, the TRDV2 antigen binding fragment is human. In some embodiments, a TRDV2 antigen binding fragment is humanized. In certain embodiments, a TRDV2 antigen binding fragment is an isolated TRDV2 antigen binding fragment.

In some embodiments, the antigen binding fragment is a diabody. In some embodiments, the antigen binding fragment is a Fab. In some embodiments, the antigen binding fragment is a Fab′. In some embodiments, the antigen binding fragment is a F(ab′)₂. In some embodiments, the antigen binding fragment is a Fv fragment. In some embodiments, the antigen binding fragment is a disulfide stabilized Fv fragment (dsFv). In some embodiments, the antigen binding fragment is a (dsFv)₂. In some embodiments, the antigen binding fragment is a bispecific dsFv (dsFv-dsFv′). In some embodiments, the antigen binding fragment is a disulfide stabilized diabody (ds diabody). In some embodiments, the antigen binding fragment is a single-chain antibody molecule (scFv). In some embodiments, the antigen binding fragment is a single domain antibody (sdAb). In some embodiments, the antigen binding fragment is an scFv dimer (bivalent diabody). In some embodiments, the antigen binding fragment is a multispecific antibody formed from a portion of an antibody comprising one or more CDRs. In some embodiments, the antigen binding fragment is a camelized single domain antibody. In some embodiments, the antigen binding fragment is a nanobody. In some embodiments, the antigen binding fragment is a domain antibody. In some embodiments, the antigen binding fragment is a bivalent domain antibody. In some embodiments, the antigen binding fragment is an antibody fragment that binds to an antigen but does not comprise a complete antibody structure.

In some embodiments, the TRDV2 antibody is a multispecific antibody. In other embodiments, the TRDV2 antibody is a bispecific antibody. In certain embodiments, the multispecific antibody comprises an antigen binding fragment of a TRDV2 antibody provided herein. In other embodiments, the bispecific antibody comprises an antigen binding fragment of a TRDV2 antibody provided herein. In some embodiments, the TRDV2 antibody is an agonistic antibody. In certain embodiments, the TRDV2 antibody activates T cells. In other embodiments, the TRDV2 antibody is an antagonistic antibody. In certain embodiments, the TRDV2 antibody inactivates T cells. In some embodiments, the TRDV2 antibody blocks activation of T cells. In some embodiments, the TRDV2 antibody modulates the activity of T cells. In some embodiments, the TRDV2 antibody neither activates or inactivates the activity of γδ T cells. In a specific embodiment, the T cells are γδ T cells.

In specific embodiments, the γδ T cells are human γδ T cells. In specific embodiments, provided is a bispecific antibody comprising a TRDV2 antibody provided herein in a knob-in-hole format. In some embodiments, a TRDV2 antibody provided herein may be comprised in a bispecific antibody. In some embodiments, a TRDV2 bispecific antibody provided herein may be comprised in a multispecific antibody. In certain embodiments, a bispecific antibody provided herein comprises a first binding domain comprising a TRDV2 antibody provided herein that binds to a first TRDV2 epitope, and a second binding domain comprising a TRDV2 antibody provided herein that binds to a second TRDV2 epitope, wherein the first TRDV2 epitope and the second TRDV2 epitope are not the same. In a specific embodiment, a TRDV2 antibody, or antigen binding fragment thereof, provided herein specifically binds to TRDV2. In certain embodiments, a TRDV2 antibody, or antigen binding fragment thereof, provided herein does not bind to an epitope of Vδ2.

In some embodiments, the VH CDR1, VH CDR2, VH CDR3, VL CDR1, VL CDR2, and VL CDR3 sequences are according to the Kabat numbering system. In some embodiments, the VH CDR1, VH CDR2, VH CDR3, VL CDR1, VL CDR2, and VL CDR3 sequences are according to the Chothia numbering system. In some embodiments, the VH CDR1, VH CDR2, VH CDR3, VL CDR1, VL CDR2, and VL CDR3 sequences are according to the Exemplary numbering system. In some embodiments, the VH CDR1, VH CDR2, VH CDR3, VL CDR1, VL CDR2, and VL CDR3 sequences are according to the Contact numbering system. In some embodiments, the VH CDR1, VH CDR2, VH CDR3, VL CDR1, VL CDR2, and VL CDR3 sequences are according to the IMGT numbering system. In some embodiments, the VH CDR1, VH CDR2, VH CDR3, VL CDR1, VL CDR2, and VL CDR3 sequences are according to the AbM numbering system. Exemplary sets of 6 CDRs (VH CDR1-3 and VL CDR1-3) of certain antibody embodiments are provided herein. Other sets of CDRs are contemplated and within the scope of the antibody embodiments provided herein.

In some embodiments, the TRDV2 antibody is a multispecific antibody. In other embodiments, the TRDV2 antibody is a bispecific antibody. In certain embodiments, the multispecific antibody comprises an antigen binding fragment of a TRDV2 antibody provided herein. In some embodiments, the multispecific antibody comprises a first binding domain that binds to a first TRDV2 epitope and a second domain that binds to a second TRDV2 epitope, wherein the first TRDV2 epitope and the second TRDV2 epitope are different. In certain embodiments, the multispecific antibody further comprises a third binding domain that binds to a target that is not TRDV2. In some embodiments, the multispecific antibody comprises heavy chain variable regions and light chain variable region. In some embodiments, the first binding domain comprises a heavy chain variable region and a light chain variable region. In some embodiments, the second binding domain comprises a heavy chain variable region and a light chain variable region. In some embodiments, the first binding domain comprises a heavy chain variable region and a light chain variable region, and the second binding domain comprises a heavy chain variable region and a light chain variable region. In some embodiments, the first binding domain of the TRDV2 antibody is not a single domain antibody or nanobody. In a some embodiments, the second binding domain of the TRDV2 antibody is not a single domain antibody or nanobody.

According to a particular aspect, provided herein is an isolated TRDV2 antibody or antigen-binding fragment thereof comprising (a) a HC1; (b) a HC2; (c) a LC1; and (d) a LC2. The HC1 can be associated with the LC1 and the HC2 can be associated with LC2. The HC1 can comprise a HCDR1, HCDR2, and HCDR3 comprising the amino acid sequences of SEQ ID NO:1, SEQ ID NO:2, and SEQ ID NO: 3, respectively, and LC1 can comprise a LCDR1, LCDR2, and LCDR3 comprising the amino acid sequences of SEQ ID NO:4, SEQ ID NO:5, and SEQ ID NO: 6, respectively. The HC1 and LC1 form a binding site for a first antigen, and the HC2 and LC2 form a binding site for a second antigen. The binding site for a first antigen can, for example, bind a TRDV2 on a γδ T cell.

Also provided herein are anti-TRDV2 bispecific antibodies or antigen-binding fragments thereof comprising an anti-TRDV2 antibody or an antigen-binding fragment thereof and an antibody or antigen-binding fragment thereof that binds to a second target antigen. In certain embodiments the anti-TRDV2 bispecific antibody or antigen binding fragment thereof comprises (a) a HC1; (b) a HC2; (c) a LC1; and (d) a LC2, wherein HC1 is associated with LC1 and HC2 is associated with LC2. HC1 can, for example, comprise a HCDR1, HCDR2, and HCDR3 comprising the amino acid sequences of SEQ ID NO:1, SEQ ID NO:2, and SEQ ID NO:3, respectively, and LC1 can, for example, comprise a LCDR1, LCDR2, and LCDR3 comprising the amino acid sequences of SEQ ID NO:4, SEQ ID NO:5, and SEQ ID NO: 6, respectively, to form a binding site for a first antigen that specifically binds TRDV2. HC2 can, for example, comprise a HCDR1, HCDR2, and HCDR3, and LC2 can, for example, comprise a LCDR1, LCDR2, and LCDR3 to form a binding site for the second target antigen that specifically binds the second target antigen. In certain embodiments, the TRDV2 is on the surface of a γδ T cell. In certain embodiments, the second target antigen is on the surface of a second target cell. In some embodiments, The binding of the TRDV2 bispecific antibody to TRDV2 present on the surface of the γδ T cell, and the binding of the second target antigen present on the surface of the second target cell can, for example, result in the killing of the second cell.

In certain embodiments, the anti-TRDV2 antibodies or antigen binding fragments thereof binds to a first epitope located on TRDV2 and a second epitope of a cancer cell.

In some embodiments, provided herein is a bispecific antibody comprising: (a) a first binding domain that binds to a TRDV2 antigen, and (b) a second binding domain that binds to a cancer cell antigen. In some embodiments, provided herein is a bispecific antibody comprising: (a) a first binding domain that specifically binds to a TRDV2 antigen, and (b) a second binding domain that specifically binds to a cancer cell antigen.

In some embodiments, provided herein is a bispecific antibody comprising: (a) a first binding domain that binds to a first epitope on a TRDV2 antigen, and (b) a second binding domain that binds to a second epitope on a cancer cell antigen. In some embodiments, provided herein is a bispecific antibody comprising: (a) a first binding domain that specifically binds to a first epitope on a TRDV2 antigen, and (b) a second binding domain that specifically binds to a second epitope on a cancer cell antigen.

In an embodiment of the bispecific antibodies provided herein, the first epitope is located on TRDV2 and the second epitope is located on the surface of a cancer cell. In some embodiments, the second epitope is located on a cancer cell antigen. In an embodiment of the bispecific antibodies provided herein, the first epitope is located on TRDV2 and the second epitope is located on a tumor. In an embodiment of the bispecific antibodies provided herein, the first epitope is located on TRDV2 and the second epitope is located on a tumor-specific antigen. In an embodiment of the bispecific antibodies provided herein, the first epitope is located on TRDV2 and the second epitope is located on a tumor associated antigen. In an embodiment of the bispecific antibodies provided herein, the first epitope is located on TRDV2 and the second epitope is located on a neoantigen.

In some embodiments, the cancer cell is a cell of an adrenal cancer, anal cancer, appendix cancer, bile duct cancer, bladder cancer, bone cancer, brain cancer, breast cancer, cervical cancer, colorectal cancer, esophageal cancer, gallbladder cancer, gestational trophoblastic, head and neck cancer, Hodgkin lymphoma, intestinal cancer, kidney cancer, leukemia, liver cancer, lung cancer, melanoma, mesothelioma, multiple myeloma, neuroendocrine tumor, non-Hodgkin lymphoma, oral cancer, ovarian cancer, pancreatic cancer, prostate cancer, sinus cancer, skin cancer, soft tissue sarcoma spinal cancer, stomach cancer, testicular cancer, throat cancer, thyroid cancer, uterine cancer endometrial cancer, vaginal cancer, or vulvar cancer. In some embodiments, the cancer is an adrenal cancer, anal cancer, appendix cancer, bile duct cancer, bladder cancer, bone cancer, brain cancer, breast cancer, cervical cancer, colorectal cancer, esophageal cancer, gallbladder cancer, gestational trophoblastic, head and neck cancer, Hodgkin lymphoma, intestinal cancer, kidney cancer, leukemia, liver cancer, lung cancer, melanoma, mesothelioma, multiple myeloma, neuroendocrine tumor, non-Hodgkin lymphoma, oral cancer, ovarian cancer, pancreatic cancer, prostate cancer, sinus cancer, skin cancer, soft tissue sarcoma spinal cancer, stomach cancer, testicular cancer, throat cancer, thyroid cancer, uterine cancer endometrial cancer, vaginal cancer, or vulvar cancer. In some embodiments, the cancer is a adrenal cancer. In some embodiments, the cancer is a anal cancer. In some embodiments, the cancer is an appendix cancer. In some embodiments, the cancer is a bile duct cancer. In some embodiments, the cancer is a bladder cancer. In some embodiments, the cancer is a bone cancer. In some embodiments, the cancer is a brain cancer. In some embodiments, the cancer is a breast cancer. In some embodiments, the cancer is a cervical cancer. In some embodiments, the cancer is a colorectal cancer. In some embodiments, the cancer is a esophageal cancer. In some embodiments, the cancer is a gallbladder cancer. In some embodiments, the cancer is a gestational trophoblastic. In some embodiments, the cancer is a head and neck cancer. In some embodiments, the cancer is a Hodgkin lymphoma. In some embodiments, the cancer is an intestinal cancer. In some embodiments, the cancer is a kidney cancer. In some embodiments, the cancer is a leukemia. In some embodiments, the cancer is a liver cancer. In some embodiments, the cancer is a lung cancer. In some embodiments, the cancer is a melanoma. In some embodiments, the cancer is a mesothelioma. In some embodiments, the cancer is a multiple myeloma. In some embodiments, the cancer is a neuroendocrine tumor. In some embodiments, the cancer is a non-Hodgkin lymphoma. In some embodiments, the cancer is an oral cancer. In some embodiments, the cancer is a ovarian cancer. In some embodiments, the cancer is a pancreatic cancer. In some embodiments, the cancer is a prostate cancer. In some embodiments, the cancer is a sinus cancer. In some embodiments, the cancer is a skin cancer. In some embodiments, the cancer is a soft tissue sarcoma spinal cancer. In some embodiments, the cancer is a stomach cancer. In some embodiments, the cancer is a testicular cancer. In some embodiments, the cancer is a throat cancer. In some embodiments, the cancer is a thyroid cancer. In some embodiments, the cancer is a uterine cancer endometrial cancer. In some embodiments, the cancer is a vaginal cancer. In some embodiments, the cancer is a vulvar cancer.

In some embodiments, the adrenal cancer is an adrenocortical carcinoma (ACC), adrenal cortex cancer, pheochromocytoma, or neuroblastoma.

In some embodiments, the anal cancer is a squamous cell carcinoma, cloacogenic carcinoma, adenocarcinoma, basal cell carcinoma, or melanoma.

In some embodiments, the appendix cancer is a neuroendocrine tumor (NET), mucinous adenocarcinoma, goblet cell carcinoid, intestinal-type adenocarcinoma, or signet-ring cell adenocarcinoma.

In some embodiments, the bile duct cancer is an extrahepatic bile duct cancer, adenocarcinomas, hilar bile duct cancer, perihilar bile duct cancer, distal bile duct cancer, or intrahepatic bile duct cancer.

In some embodiments, the bladder cancer is transitional cell carcinoma (TCC), papillary carcinoma, flat carcinoma, squamous cell carcinoma, adenocarcinoma, small-cell carcinoma, or sarcoma.

In some embodiments, the bone cancer is a primary bone cancer, sarcoma, osteosarcoma, chondrosarcoma, sarcoma, fibrosarcoma, malignant fibrous histiocytoma, giant cell tumor of bone, chordoma, or metastatic bone cancer.

In some embodiments, the brain cancer is an astrocytoma, brain stem glioma, glioblastoma, meningioma, ependymoma, oligodendroglioma, mixed glioma, pituitary carcinoma, pituitary adenoma, craniopharyngioma, germ cell tumor, pineal region tumor, medulloblastoma, or primary CNS lymphoma.

In some embodiments, the breast cancer is a breast adenocarcinoma, invasive breast cancer, noninvasive breast cancer, breast sarcoma, metaplastic carcinoma, adenocystic carcinoma, phyllodes tumor, angiosarcoma, HER2-positive breast cancer, triple-negative breast cancer, or inflammatory breast cancer.

In some embodiments, the cervical cancer is a squamous cell carcinoma, or adenocarcinoma.

In some embodiments, the colorectal cancer is a colorectal adenocarcinoma, primary colorectal lymphoma, gastrointestinal stromal tumor, leiomyosarcoma, carcinoid tumor, mucinous adenocarcinoma, signet ring cell adenocarcinoma, gastrointestinal carcinoid tumor, or melanoma.

In some embodiments, the esophageal cancer is an adenocarcinoma or squamous cell carcinoma.

In some embodiments, the gall bladder cancer is an adenocarcinoma, papillary adenocarcinoma, adenosquamous carcinoma, squamous cell carcinoma, small cell carcinoma, or sarcoma.

In some embodiments, the gestational trophoblastic disease (GTD) is a hydatidiform mole, gestational trophoblastic neoplasia (GTN), choriocarcinoma, placental-site trophoblastic tumor (PSTT), or epithelioid trophoblastic tumor (ETT).

In some embodiments, the head and neck cancer is a laryngeal cancer, nasopharyngeal cancer, hypopharyngeal cancer, nasal cavity cancer, paranasal sinus cancer, salivary gland cancer, oral cancer, oropharyngeal cancer, or tonsil cancer.

In some embodiments, the Hodgkin lymphoma is a classical Hodgkin lymphoma, nodular sclerosis, mixed cellularity, lymphocyte-rich, lymphocyte-depleted, or nodular lymphocyte-predominant Hodgkin lymphoma (NLPHL).

In some embodiments, the intestinal cancer is a small intestine cancer, small bowel cancer, adenocarcinoma, sarcoma, gastrointestinal stromal tumors, carcinoid tumors, or lymphoma.

In some embodiments, the kidney cancer is a renal cell carcinoma (RCC), clear cell RCC, papillary RCC, chromophobe RCC, collecting duct RCC, unclassified RCC, transitional cell carcinoma, urothelial cancer, renal pelvis carcinoma, or renal sarcoma.

In some embodiments, the leukemia is an acute lymphocytic leukemia (ALL), acute myeloid leukemia (AML), chronic lymphocytic leukemia (CLL), chronic myeloid leukemia (CML), hairy cell leukemia (HCL), or a myelodysplastic syndrome (MDS). In a specific embodiment, the leukemia is AML.

In some embodiments, the liver cancer is a hepatocellular carcinoma (HCC), fibrolamellar HCC, cholangiocarcinoma, angiosarcoma, or liver metastasis.

In some embodiments, the lung cancer is a small cell lung cancer, small cell carcinoma, combined small cell carcinoma, non-small cell lung cancer, lung adenocarcinoma, squamous cell lung cancer, large-cell undifferentiated carcinoma, pulmonary nodule, metastatic lung cancer, adenosquamous carcinoma, large cell neuroendocrine carcinoma, salivary gland-type lung carcinoma, lung carcinoid, mesothelioma, sarcomatoid carcinoma of the lung, or malignant granular cell lung tumor.

In some embodiments, the melanoma is a superficial spreading melanoma, nodular melanoma, acral-lentiginous melanoma, lentigo maligna melanoma, amelanotic melanoma, desmoplastic melanoma, ocular melanoma, or metastatic melanoma.

In some embodiments, the mesothelioma is a pleural mesothelioma, peritoneal mesothelioma, pericardial mesothelioma, or testicular mesothelioma.

In some embodiments, the multiple myeloma is an active myeloma or smoldering myeloma.

In some embodiments, the neuroendocrine tumor, is a gastrointestinal neuroendocrine tumor, pancreatic neuroendocrine tumor, or lung neuroendocrine tumor.

In some embodiments, the non-Hodgkin's lymphoma is an anaplastic large-cell lymphoma, lymphoblastic lymphoma, peripheral T cell lymphoma, follicular lymphoma, cutaneous T cell lymphoma, lymphoplasmacytic lymphoma, marginal zone B-cell lymphoma, MALT lymphoma, small-cell lymphocytic lymphoma, Burkitt lymphoma, chronic lymphocytic leukemia (CLL), small lymphocytic lymphoma (SLL), precursor T-lymphoblastic leukemia/lymphoma, acute lymphocytic leukemia (ALL), adult T cell lymphoma/leukemia (ATLL), hairy cell leukemia, B-cell lymphomas, diffuse large B-cell lymphoma (DLBCL), primary mediastinal B-cell lymphoma, primary central nervous system (CNS) lymphoma, mantle cell lymphoma (MCL), marginal zone lymphomas, mucosa-associated lymphoid tissue (MALT) lymphoma, nodal marginal zone B-cell lymphoma, splenic marginal zone B-cell lymphoma, lymphoplasmacytic lymphoma, B-cell non-Hodgkin lymphoma, T cell non-Hodgkin lymphoma, natural killer cell lymphoma, cutaneous T cell lymphoma, Alibert-Bazin syndrome, Sezary syndrome, primary cutaneous anaplastic large-cell lymphoma, peripheral T cell lymphoma, angioimmunoblastic T cell lymphoma (AITL), anaplastic large-cell lymphoma (ALCL), systemic ALCL, enteropathy-type T cell lymphoma (EATL), or hepatosplenic gamma/delta T cell lymphoma.

In a specific embodiment, the cancer is multiple myeloma (MM). In another specific embodiment, the cancer is chronic lymphocytic leukemia. In other embodiments, the cancer is acute B-lymphoblastic leukemia. In yet other embodiments, the cancer is non-Hodgkin lymphoma (NHL). In some embodiments, the cancer is Hodgkin lymphoma. In some embodiments, the oral cancer is a squamous cell carcinoma, verrucous carcinoma, minor salivary gland carcinomas, lymphoma, benign oral cavity tumor, cosinophilic granuloma, fibroma, granular cell tumor, karatoacanthoma, leiomyoma, osteochondroma, lipoma, schwannoma, neurofibroma, papilloma, condyloma acuminatum, verruciform xanthoma, pyogenic granuloma, rhabdomyoma, odontogenic tumors, leukoplakia, erythroplakia, squamous cell lip cancer, basal cell lip cancer, mouth cancer, gum cancer, or tongue cancer.

In some embodiments, the ovarian cancer is a ovarian epithelial cancer, mucinous epithelial ovarian cancer, endometrioid epithelial ovarian cancer, clear cell epithelial ovarian cancer, undifferentiated epithelial ovarian cancer, ovarian low malignant potential tumors, primary peritoneal carcinoma, fallopian tube cancer, germ cell tumors, teratoma, dysgerminoma ovarian germ cell cancer, endodermal sinus tumor, sex cord-stromal tumors, sex cord-gonadal stromal tumor, ovarian stromal tumor, granulosa cell tumor, granulosa-theca tumor, Sertoli-Leydig tumor, ovarian sarcoma, ovarian carcinosarcoma, ovarian adenosarcoma, ovarian leiomyosarcoma, ovarian fibrosarcoma, Krukenberg tumor, or ovarian cyst.

In some embodiments, the pancreatic cancer is a pancreatic exocrine gland cancer, pancreatic endocrine gland cancer, or pancreatic adenocarcinoma, islet cell tumor, or neuroendocrine tumor.

In some embodiments, the prostate cancer is a prostate adenocarcinoma, prostate sarcoma, transitional cell carcinoma, small cell carcinoma, or neuroendocrine tumor.

In some embodiments, the sinus cancer is a squamous cell carcinoma, mucosa cell carcinoma, adenoid cystic cell carcinoma, acinic cell carcinoma, sinonasal undifferentiated carcinoma, nasal cavity cancer, paranasal sinus cancer, maxillary sinus cancer, ethmoid sinus cancer, or nasopharynx cancer.

In some embodiments, the skin cancer is a basal cell carcinoma, squamous cell carcinoma, melanoma, Merkel cell carcinoma, Kaposi sarcoma (KS), actinic keratosis, skin lymphoma, or keratoacanthoma.

In some embodiments, the soft tissue cancer is an angiosarcoma, dermatofibrosarcoma, epithelioid sarcoma, Ewing's sarcoma, fibrosarcoma, gastrointestinal stromal tumors (GISTs), Kaposi sarcoma, leiomyosarcoma, liposarcoma, dedifferentiated liposarcoma (DL), myxoid/round cell liposarcoma (MRCL), well-differentiated liposarcoma (WDL), malignant fibrous histiocytoma, neurofibrosarcoma, rhabdomyosarcoma (RMS), or synovial sarcoma.

In some embodiments, the spinal cancer is a spinal metastatic tumor.

In some embodiments, the stomach cancer is a stomach adenocarcinoma, stomach lymphoma, gastrointestinal stromal tumors, carcinoid tumor, gastric carcinoid tumors, Type I ECL-cell carcinoid, Type II ECL-cell carcinoid, or Type III ECL-cell carcinoid.

In some embodiments, the testicular cancer is a seminoma, non-seminoma, embryonal carcinoma, yolk sac carcinoma, choriocarcinoma, teratoma, gonadal stromal tumor, leydig cell tumor, or sertoli cell tumor.

In some embodiments, the throat cancer is a squamous cell carcinoma, adenocarcinoma, sarcoma, laryngeal cancer, pharyngeal cancer, nasopharynx cancer, oropharynx cancer, hypopharynx cancer, laryngeal cancer, laryngeal squamous cell carcinoma, laryngeal adenocarcinoma, lymphoepithelioma, spindle cell carcinoma, verrucous cancer, undifferentiated carcinoma, or lymph node cancer.

In some embodiments, the thyroid cancer is a papillary carcinoma, follicular carcinoma, Hürthle cell carcinoma, medullary thyroid carcinoma, or anaplastic carcinoma.

In some embodiments, the uterine cancer is an endometrial cancer, endometrial adenocarcinoma, endometroid carcinoma, serous adenocarcinoma, adenosquamous carcinoma, uterine carcinosarcoma, uterine sarcoma, uterine leiomyosarcoma, endometrial stromal sarcoma, or undifferentiated sarcoma.

In some embodiments, the vaginal cancer is a squamous cell carcinoma, adenocarcinoma, melanoma, or sarcoma.

In some embodiments, the vulvar cancer is a squamous cell carcinoma or adenocarcinoma.

In one embodiment, the cancer is a solid cancer. In one embodiment, the cancer is a solid tumor. In one embodiment, the cancer is a liquid cancer. In one embodiment, the cancer is a liquid tumor. In some embodiments, the cancer is a hematologic malignancy. In certain embodiments, the cancer is benign. In some embodiments, the cancer is malignant. In some embodiments, the cancer is metastatic.

In some embodiments, the second epitope is located on a cancer antigen.

In some embodiments, the cancer antigen is angiopoietin, BCMA, CD19, CD20, CD22, CD25 (IL2-R), CD30, CD33, CD37, CD38, CD52, CD56, CD123 (IL-3R), cMET, DLL/Notch, EGFR, EpCAM, FGF, FGF-R, GD2, HER2, Mesothelin, Nectin-4, prostatic acid phosphatase (PAP), PDGFRα, prostate-specific antigen (PSA), PSA3, prostate-specific membrane antigen (PSMA), RANKL, SLAMF7, STEAP1, T cell receptor gamma alternate reading frame protein (TARP), TROP2, VEGF, or VEGF-R. In some embodiments, the cancer antigen is angiopoietin. In some embodiments, the cancer antigen is BCMA. In some embodiments, the cancer antigen is CD19. In some embodiments, the cancer antigen is CD20. In some embodiments, the cancer antigen is CD22. In some embodiments, the cancer antigen is CD25 (IL2-R). In some embodiments, the cancer antigen is CD30. In some embodiments, the cancer antigen is CD33. In some embodiments, the cancer antigen is CD37. In some embodiments, the cancer antigen is CD38. In some embodiments, the cancer antigen is CD52. In some embodiments, the cancer antigen is CD56. In some embodiments, the cancer antigen is CD123 (IL-3R). In some embodiments, the cancer antigen is cMET. In some embodiments, the cancer antigen is DLL/Notch. In some embodiments, the cancer antigen is EGFR. In some embodiments, the cancer antigen is EpCAM. In some embodiments, the cancer antigen is FGF. In some embodiments, the cancer antigen is FGF-R. In some embodiments, the cancer antigen is GD2. In some embodiments, the cancer antigen is HER2. In some embodiments, the cancer antigen is Mesothelin. In some embodiments, the cancer antigen is Nectin-4. In some embodiments, the cancer antigen is PAP. In some embodiments, the cancer antigen is PDGFRα. In some embodiments, the cancer antigen is PSA. In some embodiments, the cancer antigen is PSA3. In some embodiments, the cancer antigen is PSCA. In some embodiments, the cancer antigen is PSMA. In some embodiments, the cancer antigen is RANKL. In some embodiments, the cancer antigen is SLAMF7. In some embodiments, the cancer antigen is STEAP1. In some embodiments, the cancer antigen is TARP. In some embodiments, the cancer antigen is TROP2. In some embodiments, the cancer antigen is VEGF. In some embodiments, the cancer antigen is VEGF-R.

In some embodiments, the cancer antigen is CEA, immature laminin receptor, TAG-72, HPV E6, HPV E7, BING-4, calcium-activated chloride channel 2, cyclin-B1, 9D7, EpCAM, EphA3, Her2/neu, telomerase, mesothelin, SAP-1, surviving, a BAGE family antigen, CAGE family antigen, GAGE family antigen, MAGE family antigen, SAGE family antigen, XAGE family antigen, NY-ESO-1/LAGE-1, PRAME, SSX-2, Melan-A, MART-1, Gp100, pmel17, tyrosinase, TRP-1, TRP-2, P. polypeptide, MCIR, prostate-specific antigen, β-catenin, BRCA1, BRCA2, CDK4, CML66, fibronectin, MART-2, p53, Ras, TGF-βRII, or MUC1. In some embodiments, the cancer antigen is CEA. In some embodiments, the cancer antigen is immature laminin receptor. In some embodiments, the cancer antigen is TAG-72. In some embodiments, the cancer antigen is HPV E6. In some embodiments, the cancer antigen is HPV E7. In some embodiments, the cancer antigen is BING-4. In some embodiments, the cancer antigen is calcium-activated chloride channel 2. In some embodiments, the cancer antigen is cyclin-B1. In some embodiments, the cancer antigen is 9D7. In some embodiments, the cancer antigen is EpCAM. In some embodiments, the cancer antigen is EphA3. In some embodiments, the cancer antigen is Her2/neu. In some embodiments, the cancer antigen is telomerase. In some embodiments, the cancer antigen is mesothelin. In some embodiments, the cancer antigen is SAP-1. In some embodiments, the cancer antigen is surviving. In some embodiments, the cancer antigen is a BAGE family antigen. In some embodiments, the cancer antigen is CAGE family antigen. In some embodiments, the cancer antigen is GAGE family antigen. In some embodiments, the cancer antigen is MAGE family antigen. In some embodiments, the cancer antigen is SAGE family antigen. In some embodiments, the cancer antigen is XAGE family antigen. In some embodiments, the cancer antigen is NY-ESO-1/LAGE-1. In some embodiments, the cancer antigen is PRAME. In some embodiments, the cancer antigen is SSX-2. In some embodiments, the cancer antigen is Melan-A. In some embodiments, the cancer antigen is MART-1. In some embodiments, the cancer antigen is Gp100. In some embodiments, the cancer antigen is pmel17. In some embodiments, the cancer antigen is tyrosinase. In some embodiments, the cancer antigen is TRP-1. In some embodiments, the cancer antigen is TRP-2. In some embodiments, the cancer antigen is P. polypeptide. In some embodiments, the cancer antigen is MC1R. In some embodiments, the cancer antigen is prostate-specific antigen. In some embodiments, the cancer antigen is β-catenin. In some embodiments, the cancer antigen is BRCA1. In some embodiments, the cancer antigen is BRCA2. In some embodiments, the cancer antigen is CDK4. In some embodiments, the cancer antigen is CML66. In some embodiments, the cancer antigen is fibronectin. In some embodiments, the cancer antigen is MART-2. In some embodiments, the cancer antigen is p53. In some embodiments, the cancer antigen is Ras. In some embodiments, the cancer antigen is TGF-βRII. In some embodiments, the cancer antigen is MUC1.

The binding of the TRDV2 bispecific antibody to TRDV2 present on the surface of the γδ T cell, and the binding of the tumor associated antigen present on the surface of the cancer cell can, for example, result in the killing of the cancer cell.

In an embodiment of the bispecific antibodies provided herein, the first epitope is located on TRDV2 and the second epitope is located on CD33. In certain embodiments, the second epitope comprises an epitope in the C2 domain of CD33. In other embodiments, the second epitope comprises an epitope in the V domain of CD33. In certain embodiments, the second epitope consists of an epitope in the C2 domain of CD33. In other embodiments, the second epitope consists of an epitope in the V domain of CD33.

In an embodiment of the bispecific antibodies provided herein, the first epitope is located on TRDV2 and the second epitope is located on PD-1, PD-L1, CTLA-4, EGFR, HER-2, CD19, CD20, CD3 and/or other cancer associated immune suppressors or surface antigens.

The term “CD33” refers to a 67 kD single pass transmembrane glycoprotein and is a member of the sialic acid-binding immunoglobulin-like lectins (Siglecs) family. While its exact biological function is unclear, in normal individuals, it is primarily considered to be a myeloid differentiation antigen, with low expression in myeloid progenitors, neutrophils and macrophages while being highly expressed in circulating monocytes and dendritic cells. CD33 has been detected on blasts and leukemic stem cells of 85-90% of patients presenting with in acute myeloid leukemia (AML). The term “CD33” includes any CD33 variant, isoform, and species homolog, which is naturally expressed by cells or can be expressed on cells transfected with genes or cDNA encoding those polypeptides, unless noted, the “CD33” is a human CD33. A human CD33 amino acid sequence is provided by GenBank Accession Number BC028152.1.

Also provided herein are anti-TRDV2/anti-CD33 bispecific antibodies or antigen-binding fragments thereof comprising an anti-TRDV2 antibody or an antigen-binding fragment thereof and an anti-CD33 antibody or antigen-binding fragment thereof. In certain embodiments the anti-TRDV2/anti-CD33 bispecific antibody or antigen binding fragment thereof comprises (a) a HC1; (b) a HC2; (c) a LC1; and (d) a LC2, wherein HC1 is associated with LC1 and HC2 is associated with LC2. HC1 can, for example, comprise a HCDR1, HCDR2, and HCDR3 comprising the amino acid sequences of SEQ ID NO:1, SEQ ID NO:2, and SEQ ID NO:3, respectively, and LC1 can, for example, comprise a LCDR1, LCDR2, and LCDR3 comprising the amino acid sequences of SEQ ID NO:4, SEQ ID NO:5, and SEQ ID NO: 6, respectively, to form a binding site for a first antigen that specifically binds TRDV2. HC2 can, for example, comprise a HCDR1, HCDR2, and HCDR3 comprising the amino acid sequences of SEQ ID NO:9, SEQ ID NO: 10, and SEQ ID NO: 11, respectively, and LC2 can, for example, comprise a LCDR1, LCDR2, and LCDR3 comprising the amino acid sequences of SEQ ID NO: 12, SEQ ID NO:13, and SEQ ID NO:14, respectively, to form a binding site for a second antigen that specifically binds CD33. In certain embodiments, the TRDV2 is on the surface of a γδ T cell. In certain embodiments, the CD33 is on the surface of a cancer cell (e.g., a leukemia cell).

In certain embodiments, provided herein are anti-TRDV2/anti-CD33 bispecific antibodies or antigen-binding fragments thereof comprising an anti-TRDV2 antibody or an antigen-binding fragment thereof and an anti-CD33 C2 domain antibody or antigen-binding fragment thereof. In other embodiments, provided herein are anti-TRDV2/anti-CD33 bispecific antibodies or antigen-binding fragments thereof comprising an anti-TRDV2 antibody or an antigen-binding fragment thereof and an anti-CD33 V domain antibody or antigen-binding fragment thereof.

The binding site for a second antigen can, for example, bind a cancer antigen present on the surface of a cancer cell. The binding of the TRDV2 bispecific antibody to TRDV2 present on the surface of the γδ T cell, and the binding of the tumor associated antigen present on the surface of the cancer cell can, for example, result in the killing of the cancer cell.

Expression of CD33 tends to be restricted to hematopoietic cells but is absent on normal hematopoietic stem cells, which indicates that CD33 to target cells of AML.

The structure of CD33 consists of an amino-terminal V-set Ig-like domain (coded by exon 2 of CD33) that mediates sialic acid binding and a C2-set Ig-like domain (coded by exons 3 and 4) in its extracellular portion (Laszlo et al., 2016). Alternative splicing of CD33 RNA can lead to a shorter isoform that is expressed on the cell surface, which lacks the V—but retains the C2—set Ig-like domain (Laszlo, Estey, & Walter, 2014; Laszlo et al., 2016). The biological relevance of this splicing process was largely unknown until recent studies showed that a single nucleotide polymorphism (SNP) rs 12459419 was present in 50% of the AML population and leads to skipping of exon 2 of CD33 which results in the deletion of the V domain of CD33 (Lamba et al., 2017).

Additional CD33 antibodies that can be used for the TRDV2 multispecific antibodies provided herein include AMG330 and AMG673 (Amgen; Friedrich et al., 2014), AMV564 (Amphivena; U.S. Pat. No. 9,803,029), IMGN779 (Immunogen; U.S. Pat. No. 9,359,442), BI836858 (Boehringer Ingelheim; Vasu et al., 2016), Actimab (Actinium Pharma), gemtuzumab (Godwin, Gale, & Walter, 2017), and SGN33A (Seattle Genetics). In some embodiments of the multispecific TRDV2 antibodies provided herein, the second binding domain that binds CD33 comprises the VH CDR1-3 and VL CDR1-3 of AMG330. In some embodiments of the multispecific TRDV2 antibodies provided herein, the second binding domain that binds CD33 comprises the VH CDR1-3 and VL CDR1-3 of AMG673. In some embodiments of the multispecific TRDV2 antibodies provided herein, the second binding domain that binds CD33 comprises the VH CDR1-3 and VL CDR1-3 of AMV564. In some embodiments of the multispecific TRDV2 antibodies provided herein, the second binding domain that binds CD33 comprises the VH CDR1-3 and VL CDR1-3 of IMGN779. In some embodiments of the multispecific TRDV2 antibodies provided herein, the second binding domain that binds CD33 comprises the VH CDR1-3 and VL CDR1-3 of BI836858. In some embodiments of the multispecific TRDV2 antibodies provided herein, the second binding domain that binds CD33 comprises the VH CDR1-3 and VL CDR1-3 of Actimab. In some embodiments of the multispecific TRDV2 antibodies provided herein, the second binding domain that binds CD33 comprises the VH CDR1-3 and VL CDR1-3 of gentuzimab. In some embodiments of the multispecific TRDV2 antibodies provided herein, the second binding domain that binds CD33 comprises the VH CDR1-3 and VL CDR1-3 of SGN33A.

The TRDV2xCD33 multispecific antibody can comprise a first binding domain comprising any TRDV2 antibody provided herein. The TRDV2xCD33 multispecific antibody can further comprise a second binding domain comprising any CD33 antibody, including any CD33 antibody provided herein.

In some embodiments, the bispecific antibody provided herein is a diabody, a cross-body, or a bispecific antibody obtained via a controlled Fab arm exchange as those described herein.

In some embodiments, the bispecific antibodies include IgG-like molecules with complementary CH3 domains to force heterodimerization; recombinant IgG-like dual targeting molecules, wherein the two sides of the molecule each contain the Fab fragment or part of the Fab fragment of at least two different antibodies; IgG fusion molecules, wherein full length IgG antibodies are fused to an extra Fab fragment or parts of Fab fragment; Fc fusion molecules, wherein single chain Fv molecules or stabilized diabodies are fused to heavy-chain constant-domains, Fc-regions or parts thereof; Fab fusion molecules, wherein different Fab-fragments are fused together; ScFv- and diabody-based and heavy chain antibodies (e.g., domain antibodies, nanobodies) wherein different single chain Fv molecules or different diabodies or different heavy-chain antibodies (e.g. domain antibodies, nanobodies) are fused to each other or to another protein or carrier molecule.

In some embodiments, IgG-like molecules with complementary CH3 domains molecules include the Triomab/Quadroma (Trion Pharma/Fresenius Biotech), the Knobs-into-Holes (Genentech), CrossMAbs (Roche) and the electrostatically-matched (Amgen), the LUZ-Y (Genentech), the Strand Exchange Engineered Domain body (SEEDbody) (EMD Serono), the Biclonic (Merus) and the DuoBody (Genmab A/S).

In some embodiments, recombinant IgG-like dual targeting molecules include Dual Targeting (DT)-Ig (GSK/Domantis), Two-in-one Antibody (Genentech), Cross-linked Mabs (Karmanos Cancer Center), mAb2 (F-Star) and CovX-body (CovX/Pfizer).

In some embodiments, IgG fusion molecules include Dual Variable Domain (DVD)-Ig (Abbott), IgG-like Bispecific (InnClone/Eli Lilly), Ts2Ab (MedImmune/AZ) and BsAb (Zymogenetics), HERCULES (Biogen Idec) and TvAb (Roche).

In some embodiments, Fc fusion molecules can include ScFv/Fc Fusions (Academic Institution), SCORPION (Emergent BioSolutions/Trubion, Zymogenetics/BMS), Dual Affinity Retargeting Technology (Fc-DART) (MacroGenics) and Dual (ScFv)₂-Fab (National Research Center for Antibody Medicine—China).

In some embodiments, Fab fusion bispecific antibodies include F (ab) 2 (Medarex/AMGEN), Dual-Action or Bis-Fab (Genentech), Dock-and-Lock (DNL) (ImmunoMedics), Bivalent Bispecific (Biotecnoly and Fab-Fv (UCB-Celltech). ScFv-, diabody-based, and domain antibodies, include but are not limited to, Bispecific T Cell Engager (BITE) (Micromet), Tandem Diabody (Tandab) (Affimed), Dual Affinity Retargeting Technology (DART) (MacroGenics), Single-chain Diabody (Academic), TCR-like Antibodies (AIT, ReceptorLogics), Human Serum Albumin ScFv Fusion (Merrimack) and COMBODY (Epigen Biotech), dual targeting nanobodies (Ablynx), dual targeting heavy chain only domain antibodies.

Full length bispecific antibodies provided herein can be generated for example using Fab arm exchange (or half molecule exchange) between two mono specific bivalent antibodies by introducing substitutions at the heavy chain CH3 interface in each half molecule to favor heterodimer formation of two antibody half molecules having distinct specificity either in vitro in cell-free environment or using co-expression. The Fab arm exchange reaction is the result of a disulfide-bond isomerization reaction and dissociation-association of CH3 domains. The heavy-chain disulfide bonds in the hinge regions of the parent mono specific antibodies are reduced. The resulting free cysteines of one of the parent monospecific antibodies form an inter heavy-chain disulfide bond with cysteine residues of a second parent mono specific antibody molecule and simultaneously CH3 domains of the parent antibodies release and reform by dissociation-association. The CH3 domains of the Fab arms can be engineered to favor heterodimerization over homodimerization. The resulting product is a bispecific antibody having two Fab arms or half molecules which each binding a distinct epitope, i.e. an epitope on TRDV2 and an epitope on a second target antigen (e.g., not TRDV2). Other methods of making multispecific antibodies are known and contemplated.

“Homodimerization” as used herein refers to an interaction of two heavy chains having identical CH3 amino acid sequences. “Homodimer” as used herein refers to an antibody having two heavy chains with identical CH3 amino acid sequences.

“Heterodimerization” as used herein refers to an interaction of two heavy chains having non-identical CH3 amino acid sequences. “Heterodimer” as used herein refers to an antibody having two heavy chains with non-identical CH3 amino acid sequences.

As mentioned elsewhere, in some embodiments, the bispecific antibodies include designs such as the Triomab/Quadroma (Trion Pharma/Fresenius Biotech), Knob-in-Hole (Genentech), CrossMAbs (Roche) and the electrostatically-matched (Chugai, Amgen, NovoNordisk, Oncomed), the LUZ-Y (Genentech), the Strand Exchange Engineered Domain body (SEEDbody) (EMD Serono), the Biclonic (Merus) and the DuoBody (Genmab A/S).

In some embodiments, a TRDV2 multispecific antibody provided herein is in the knob-and-hole format. In some embodiments, a TRDV2 multispecific antibody provided herein is in a DuoBody format.

The Triomab quadroma technology can be used to generate full length bispecific antibodies provided herein. Triomab technology promotes Fab arm exchange between two parental chimeric antibodies, one parental mAb having IgG2a and the second parental mAb having rat IgG2b constant regions, yielding chimeric bispecific antibodies.

The “knob-in-hole” strategy (see, e.g., PCT Publ. No. WO2006/028936) can be used to generate full length bispecific antibodies. Briefly, selected amino acids forming the interface of the CH3 domains in human IgG can be mutated at positions affecting CH3 domain interactions to promote heterodimer formation. An amino acid with a small side chain (hole) is introduced into a heavy chain of an antibody specifically binding a first antigen and an amino acid with a large side chain (knob) is introduced into a heavy chain of an antibody specifically binding a second antigen. After co-expression of the two antibodies, a heterodimer is formed as a result of the preferential interaction of the heavy chain with a “hole” with the heavy chain with a “knob.” Exemplary CH3 substitution pairs forming a knob and a hole are (expressed as modified position in the first CH3 domain of the first heavy chain/modified position in the second CH3 domain of the second heavy chain): T366Y/F405A, T366W/F405W, F405W/Y407A, T394W/Y407T, T394S/Y407A, T366W/T394S, F405W/T394S and T366W/T366S_L368A_Y407V.

The CrossMAb technology can be used to generate full length bispecific antibodies provided herein. CrossMAbs, in addition to utilizing the “knob-in-hole” strategy to promoter Fab arm exchange, have in one of the half arms the CH1 and the CL domains exchanged to ensure correct light chain pairing of the resulting bispecific antibody (see e.g. U.S. Pat. No. 8,242,247).

Other cross-over strategies can be used to generate full length bispecific antibodies provided herein by exchanging variable or constant, or both domains between the heavy chain and the light chain or within the heavy chain in the bispecific antibodies, either in one or both arms. These exchanges include for example VH-CH1 with VL-CL, VH with VL, CH3 with CL and CH3 with CH1 as described in Int. Patent Publ. Nos. WO2009/080254, WO2009/080251, WO2009/018386 and WO2009/080252.

Other strategies such as promoting heavy chain heterodimerization using electrostatic interactions by substituting positively charged residues at one CH3 surface and negatively charged residues at a second CH3 surface can be used, as described in US Pat. Publ. No. US2010/0015133; US Pat. Publ. No. US2009/0182127; US Pat. Publ. No. US2010/028637; or US Pat. Publ. No. US2011/0123532. In other strategies, heterodimerization can be promoted by the following substitutions (expressed as modified position in the first CH3 domain of the first heavy chain/modified position in the second CH3 domain of the second heavy chain): L351Y_F405AY407V/T394W, T366I_K392M_T394W/F405A_Y407V, T366L_K392M_T394W/F405A_Y407V, L351Y_Y407A/T366A_K409F, L351Y_Y407A/T366V K409F Y407A/T366A_K409F, or T350V_L351Y_F405A Y407V/T350V_T366L_K392L_T394W as described in U.S. Pat. Publ. No. US2012/0149876 or U.S. Pat. Publ. No. US2013/0195849.

LUZ-Y technology can be utilized to generate bispecific antibodies provided herein. In this technology, a leucine zipper is added into the C terminus of the CH3 domains to drive the heterodimer assembly from parental mAbs that is removed post-purification as described in Wranik et al., (2012) J Biol Chem 287 (52): 42221-9.

SEEDbody technology can be utilized to generate bispecific antibodies provided herein. SEEDbodies have, in their constant domains, select IgG residues substituted with IgA residues to promote heterodimerszation as described in U.S. Patent No. US20070287170.

In addition to methods described above, bispecific antibodies provided herein can be generated in vitro in a cell-free environment by introducing asymmetrical mutations in the CH3 regions of two mono specific homodimeric antibodies and forming the bispecific heterodimeric antibody from two parent monospecific homodimeric antibodies in reducing conditions to allow disulfide bond isomerization according to methods described in PCT Pat. Publ. No. WO2011/131746. In the methods, the first monospecific bivalent antibody and the second monospecific bivalent antibody are engineered to have certain substitutions at the CH3 domain that promotes heterodimer stability; the antibodies are incubated together under reducing conditions sufficient to allow the cysteines in the hinge region to undergo disulfide bond isomerization; thereby generating the bispecific antibody by Fab arm exchange. The incubation conditions can optionally be restored to non-reducing conditions. Exemplary reducing agents that can be used are 2-mercaptocthylamine (2-MEA), dithiothreitol (DTT), dithioerythritol (DTE), glutathione, tris (2-carboxyethyl) phosphine (TCEP), L-cysteine and beta-mercaptoethanol, such as a reducing agent selected from the group consisting of: 2-mercaptoethylamine, dithiothreitol and tris(2-carboxyethyl) phosphine. For example, incubation for at least 90 min at a temperature of at least 20° C. in the presence of at least 25 mM 2-MEA or in the presence of at least 0.5 mM dithiothreitol at a pH from 5-8, for example at pH of 7.0 or at pH of 7.4 can be used.

In some embodiments, TRDV2 is present on the surface of a γδ T cell. In some embodiments, the TRDV2 is present on the surface of a γδ T cell, and the antigen expressed on the surface of the cancer cell is a cancer antigen. In some embodiments, the cancer cell is killed when the bispecific antibody binds to the TRDV2 on the surface of the γδ T cell and the antigen on the surface of the cancer cell. In some embodiments, the bispecific antibody induces γδ T cell dependent cytotoxicity of the cancer cell in vitro with an EC₅₀ of less than about 500 pM. In some embodiments, the bispecific antibody induces γδ T cell dependent cytotoxicity of the cancer cell in vitro with an EC₅₀ of less than about 300 pM. In some embodiments, the bispecific antibody induces γδ T cell dependent cytotoxicity of the cancer cell in vitro with an EC₅₀ of less than about 160 pM. In some embodiments, the EC₅₀ is assessed with a mixture of γδ T effector cells and target cells expressing the cancer antigen. In some embodiments, the effector cell to target cell ratio is about 0.01 to 1 to about 5 to 1. In some embodiments, the effector cell to target cell ratio is about 0.1 to 1 to about 2 to 1. In some embodiments, the effector cell to target cell ratio is about 1:1.

In certain embodiments, the anti-TRDV2 antibody or antigen-binding fragment thereof comprises a HCDR1, HCDR2, HCDR3, a LCDR1, LCDR2, and LCDR3, having the polypeptide sequence of SEQ ID NOs: 1, 2, 3, 4, 5, and 6, respectively; and the anti-CD33 antibody or antigen-binding fragment thereof comprises a HCDR1, HCDR2, HCDR3, a LCDR1, LCDR2, and LCDR3, having the polypeptide sequence of SEQ ID NOs: 9, 10, 11, 12, 13, and 14, respectively.

In specific embodiments, an antibody provided herein comprises a linker. In specific embodiments, the linker is a peptide linker. In some embodiments, the liker comprises a naturally occurring amino acid. Exemplary amino acids that can be included into the linker are Gly, Ser Pro, Thr, Glu, Lys, Arg, Ile, Leu, His and The. In some embodiments, the linker has a length that is adequate to link the VH and the VL in such a way that they form the correct conformation relative to one another so that they retain the desired activity, such as binding to the target (e.g., TRDV2).

In certain embodiments, the linker is about 5-50 amino acids long. In some embodiments, the linker is about 10-40 amino acids long. In some embodiments, the linker is about 10-35 amino acids long. In some embodiments, the linker is about 10-30 amino acids long. In some embodiments, the linker is about 10-25 amino acids long. In some embodiments, the linker is about 10-20 amino acids long. In some embodiments, the linker is about 15-20 amino acids long. In some embodiments, the linker is 6 amino acids long. In some embodiments, the linker is 7 amino acids long. In some embodiments, the linker is 8 amino acids long. In some embodiments, the linker is 9 amino acids long. In some embodiments, the linker is 10 amino acids long. In some embodiments, the linker is 11 amino acids long. In some embodiments, the linker is 12 amino acids long. In some embodiments, the linker is 13 amino acids long. In some embodiments, the linker is 14 amino acids long. In some embodiments, the linker is 15 amino acids long. In some embodiments, the linker is 16 amino acids long. In some embodiments, the linker is 17 amino acids long. In some embodiments, the linker is 18 amino acids long. In some embodiments, the linker is 19 amino acids long. In some embodiments, the linker is 20 amino acids long. In some embodiments, the linker is 21 amino acids long. In some embodiments, the linker is 22 amino acids long. In some embodiments, the linker is 23 amino acids long. In some embodiments, the linker is 24 amino acids long. In some embodiments, the linker is 25 amino acids long. In some embodiments, the linker is 26 amino acids long. In some embodiments, the linker is 27 amino acids long. In some embodiments, the linker is 28 amino acids long. In some embodiments, the linker is 29 amino acids long. In some embodiments, the linker is 30 amino acids long. In some embodiments, the linker is 31 amino acids long. In some embodiments, the linker is 32 amino acids long. In some embodiments, the linker is 33 amino acids long. In some embodiments, the linker is 34 amino acids long. In some embodiments, the linker is amino acids long. In some embodiments, the linker is 36 amino acids long. In some embodiments, the linker is 37 amino acids long. In some embodiments, the linker is 38 amino acids long. In some embodiments, the linker is 39 amino acids long. In some embodiments, the linker is 40 amino acids long. Exemplary linkers that can be used are Gly rich linkers, Gly and Ser containing linkers, Gly and Ala containing linkers, Ala and Ser containing linkers, and other flexible linkers.

Any of the VH and the VL domains identified herein (e.g., those that bind TRDV2) can be engineered into scFv format. In some embodiments, the scFv format is in the VH-linker-VL orientation. In other embodiments, the scFv format is in the VL-linker-VH orientation. Any of the VH and the VL domains identified herein can also be used to generate sc (Fv)2 structures. In some embodiments, the sc (Fv)2 structure is VH-linker-VL-linker-VL-linker-VH. In some embodiments, the sc (Fv)2 structure is VH-linker-VL-linker-VH-linker-VL. In some embodiments, the sc (Fv)2 structure is VH-linker-VH-linker-VL-linker-VL. In some embodiments, the sc (Fv)2 structure is VL-linker-VH-linker-VH-linker-VL. In some embodiments, the sc (Fv)2 structure is VL-linker-VH-linker-VL-linker-VH. In some embodiments, the sc (Fv)2 structure is VL-linker-VL-linker-VH-linker-VH. In some embodiments, the scFv comprises, from the N- to C-terminus, a VH, a first linker (L1) and a VL (VH-L1-VL). In other embodiments, the scFv comprises, from the N- to C-terminus, the VL, the L1 and the VH (VL-L1-VH). In certain embodiments, antibodies provided herein comprise two linkers. In other embodiments, antibodies provided herein comprise three linkers. In yet other embodiments, antibodies provided herein comprise four or more linkers. In certain embodiments, the antibody is an antigen binding fragment thereof.

According to another particular aspect, provided herein is an isolated anti-TRDV2/anti-CD33 bispecific antibody or antigen-binding fragment thereof that induces antibody-dependent cell-mediated cytotoxicity (ADCC). The bispecific antibody or antigen-binding fragment thereof can, for example, induce ADCC in vitro.

In certain embodiments, the bispecific antibody or antigen-binding fragment thereof induces γδ T cell dependent cytotoxicity of a cancer cell in vitro with an EC₅₀ of less than about 160 pM, when assessed in vitro at an effector to target cell ratio of 1:1. In one such embodiment, the bispecific antibody is an isolated anti-TRDV2/anti-CD33 bispecific antibody or antigen-binding fragment thereof that exhibits an EC₅₀ of less than about 160 pM, when assessed in vitro with a mixture of γδ T effector cells and Kasumi3 AML target cells, where such cells are present in an effector to target cell ratio of about 1:1 and the bispecific antibody or antigen-binding fragment thereof is present at a concentration of about 30 ng/mL.

In another embodiment, the bispecific antibody is an isolated anti-TRDV2/anti-CD33 bispecific antibody or antigen-binding fragment thereof comprising (a) a HC1; (b) a HC2; (c) a LC1; and (d) a LC2, wherein HC1 is associated with LC1 and HC2 is associated with LC2, and wherein HC1 comprises a HCDR1, HCDR2, and HCDR3 comprising the amino acid sequences of SEQ ID NO: 1, SEQ ID NO:2, and SEQ ID NO:3, respectively, and LC1 comprises a LCDR1, LCDR2, and LCDR3 comprising the amino acid sequences of SEQ ID NO:4, SEQ ID NO: 5, and SEQ ID NO:6, respectively, to form a first antigen-binding site that specifically binds TRDV2, and wherein HC2 comprises a HCDR1, HCDR2, and HCDR3 comprising the amino acid sequences of SEQ ID NO:9, SEQ ID NO: 10, and SEQ ID NO:11, respectively, and LC2 comprises a LCDR1, LCDR2, and LCDR3 comprising the amino acid sequences of SEQ ID NO: 12, SEQ ID NO: 13, and SEQ ID NO: 14 respectively, to form a second antigen-binding site that specifically binds CD33, wherein the anti-TRDV2/anti-CD33 bispecific antibody or antigen-binding fragment thereof exhibits an EC₅₀ of less than about 160 pM, when assessed in vitro with a mixture of γδ T effector cells and Kasumi3 AML target cells, where such cells are present in an effector to target cell ratio of about 1:1 and the bispecific antibody or antigen-binding fragment thereof is present at a concentration of about 30 ng/mL.

In certain embodiments, the second antigen-binding site specifically binds the C2 domain of CD33. In other embodiments, the second antigen-binding site specifically binds the V domain of CD33.

In certain embodiments, the EC₅₀ is less than about 1000 pM, less than about 900 pM, less than about 800 pM, less than about 700 pM, less than about 600 pM, less than about 500 pM, less than about 400 PM, less than about 300 PM, less than about 200 pM, less than about 190 pM, less than about 180 pM, less than about 170 pM, less than about 160 pM, less than about 150 pM, less than about 140 pM, less than about 130 pM, less than about 120 pM, less than about 110 pM, less than about 100 pM, less than about 90 pM, less than about 80 pM, less than about 70 pM, less than about 60 pM, less than about 50 pM, less than about 40 pM, less than about pM, less than about 20 pM, or less than about 10 pM.

In certain embodiments, the effector to target cell ratio can, for example, be 0.01:1, 0.02:1, 0.03:1, 0.04:1, 0.05:1, 0.06:1, 0.07:1, 0.08:1, 0.09:1, 1:1, 2:1, 3:1, 4:1, 5:1, 6:1, 7:1, 8:1, 9:1, or 10:1.

In certain embodiments, the concentration of the bispecific antibody or antigen-binding fragment thereof is about 0.000005 ng/ml, about 0.00005 ng/ml, about 0.0005, about 0.005 ng/ml, about 0.01 ng/ml, about 0.02 ng/mL, about 0.03 ng/ml, about 0.04 ng/mL, about 0.05 ng/ml, about 0.06 ng/ml, about 0.07 ng/ml, about 0.08 ng/ml, about 0.09 ng/ml, about 0.1 ng/mL, about 0.5 ng/ml, about 1.0 ng/ml, about 10 ng/ml, about 20 ng/ml about, about 30 ng/ml about 40 ng/ml, about 50 ng/ml, about 60 ng/ml, about 70 ng/mL, about 80 ng/ml, about 90 ng/mL, about 100 ng/mL, or about 1000 ng/mL.

In some embodiments described herein, immune effector properties of the anti-TRDV2 bispecific antibodies provided herein can be enhanced or silenced through Fc modifications by techniques known to those skilled in the art. For example, Fc effector functions such as Clq binding, complement dependent cytotoxicity (CDC), ADCC, antibody-dependent cell-mediated phagocytosis (ADCP), down regulation of cell surface receptors (e.g., B cell receptor; BCR), etc. can be provided and/or controlled by modifying residues in the Fc responsible for these activities.

“Antibody-dependent cell-mediated cytotoxicity” or “ADCC” refers to a cell-mediated reaction in which non-specific cytotoxic cells that express Fc receptors (FcRs) (e.g. Natural Killer (NK) cells, neutrophils, and macrophages) recognize bound antibody on a target cell and subsequently cause lysis of the target cell.

The ability of antibodies to induce ADCC can be enhanced by engineering their oligosaccharide component. Human IgG1 or IgG3 are N-glycosylated at Asn297 with the majority of the glycans in the well-known biantennary G0, G0F, G1, G1F, G2 or G2F forms. Antibodies produced by non-engineered CHO cells typically have a glycan fucose content of about at least 85%. The removal of the core fucose from the biantennary complex-type oligosaccharides attached to the Fc regions enhances the ADCC of antibodies via improved FcγRIIIa binding without altering antigen binding or CDC activity. Such Abs can be achieved using different methods reported to lead to the successful expression of relatively high defucosylated antibodies bearing the biantennary complex-type of Fc oligosaccharides such as control of culture osmolality (Konno et al., Cytotechnology 64:249-65, 2012), application of a variant CHO line Lec 13 as the host cell line (Shields et al., J Biol Chem 277:26733-26740, 2002), application of a variant CHO line EB66 as the host cell line (Olivier et al., MAbs; 2 (4), 2010; Epub ahead of print; PMID: 20562582), application of a rat hybridoma cell line YB2/0 as the host cell line (Shinkawa et al., J Biol Chem 278:3466-3473, 2003), introduction of small interfering RNA specifically against the α-1,6-fucosyltrasferase (FUT8) gene (Mori et al., Biotechnol Bioeng 88:901-908, 2004), or coexpression of β-1,4-N-acetylglucosaminyltransferase III and golgi α-mannosidase II or a potent alpha-mannosidase I inhibitor, kifunensine (Ferrara et al., J Biol Chem 281:5032-5036, 2006, Ferrara et al., Biotechnol Bioeng 93:851-861, 2006; Xhou et al., Biotechnol Bioeng 99:652-65, 2008).

In some embodiments described herein, ADCC elicited by the anti-TRDV2 bispecific antibodies provided herein can also be enhanced by certain substitutions in the antibody Fc. Exemplary substitutions are for example substitutions at amino acid positions 256, 290, 298, 312, 356, 330, 333, 334, 360, 378 or 430 (residue numbering according to the EU index) as described in U.S. Pat. No. 6,737,056.

According to another particular aspect, provided herein is an isolated anti-TRDV2 bispecific antibody or antigen-binding fragment thereof, wherein the anti-TRDV2 bispecific antibody or antigen-binding fragment thereof is chimeric.

According to another particular aspect, provided herein is an isolated anti-TRDV2 bispecific antibody or antigen-binding fragment thereof, wherein the anti-TRDV2 bispecific antibody or antigen-binding fragment thereof is human or humanized.

In some embodiments, the first binding domain is human. In some embodiments, the second binding domain is human. In other embodiments, both the first binding domain and the second binding domain are human. In some embodiments, the first binding domain is humanized. In some embodiments, the second binding domain is humanized. In other embodiments, both the first binding domain and the second binding domain are humanized. In other embodiments, both the first binding domain is human and the second binding domain is humanized. In other embodiments, both the first binding domain is humanized and the second binding domain is human.

In some embodiments, the bispecific antibody is an IgG antibody. In some embodiments, the IgG antibody is an IgG1 antibody. In some embodiments, the IgG antibody is an IgG2 antibody. In some embodiments, the IgG antibody is an IgG3 antibody. In some embodiments, the IgG antibody is an IgG4 antibody. In some embodiments, the TRDV2 antibody is a TRDV2 antigen binding fragment.

In some embodiments, the bispecific antibody is multivalent. In some embodiments, the bispecific antibody is capable of binding at least three antigens. In some embodiments, the bispecific antibody is capable of binding at least five antigens.

In certain embodiments, the bispecific antibodies provided herein are part of a multispecific antibody. In some embodiments, the multispecific antibody comprises a first binding domain that binds to a TRDV2 antigen. In some embodiments, the multispecific antibody comprises a first binding domain that binds to a TRDV2 antigen and comprises a second binding domain that binds to a second target antigen, as provided herein. In certain embodiments, the multispecific antibody binds to a TRDV2 antigen, a second target antigen, and one or more additional antigens. In some embodiments of the various antibodies provided herein, the antibody binds to an epitope of a given antigen.

In one aspect, provided is a multispecific antibody comprising: (a) a first binding domain that binds to TRDV2, and (b) a second binding domain that binds to an antigen on the surface of a cancer cell. In some embodiments, the multispecific antibody is a bispecific antibody.

In some embodiments, the first binding domain comprises a VH comprising a VH CDR1, a VH CDR2, and a VH CDR3 having an amino acid sequence of a VH CDR1, a VH CDR2, and a VH CDR3, respectively, of a VH having an amino acid sequence of SEQ ID NO:7. In some embodiments, the first binding domain comprises a VL comprising a VL CDR1, a VL CDR2, and a VL CDR3 having an amino acid sequence of a VL CDR1, a VL CDR2, and a VL CDR3, respectively, of a VL having an amino acid sequence of SEQ ID NO:8. In some embodiments, the first binding domain comprises: (i) a VH comprising a VH CDR1, a VH CDR2, and a VH CDR3 having an amino acid sequence of a VH CDR1, a VH CDR2, and a VH CDR3, respectively, of a VH having an amino acid sequence of SEQ ID NO:7; and (ii) a VL comprising a VL CDR1, a VL CDR2, and a VL CDR3 having an amino acid sequence of a VL CDR1, a VL CDR2, and a VL CDR3, respectively, of a VL having an amino acid sequence of SEQ ID NO:8. In some embodiments, the VH CDR1, VH CDR2, VH CDR3, VL CDR1, VL CDR2, and VL CDR3 amino acid sequences of the first binding domain are according to the Kabat numbering system. In some embodiments, the VH CDR1, VH CDR2, VH CDR3, VL CDR1, VL CDR2, and VL CDR3 amino acid sequences of the first binding domain are according to the Chothia numbering system. In some embodiments, the VH CDR1, VH CDR2, VH CDR3, VL CDR1, VL CDR2, and VL CDR3 amino acid sequences of the first binding domain are according to the AbM numbering system. In some embodiments, the VH CDR1, VH CDR2, VH CDR3, VL CDR1, VL CDR2, and VL CDR3 amino acid sequences of the first binding domain are according to the Contact numbering system. In some embodiments, the VH CDR1, VH CDR2, VH CDR3, VL CDR1, VL CDR2, and VL CDR3 amino acid sequences of the first binding domain are according to the IMGT numbering system. In some embodiments, the VH CDR1, VH CDR2, VH CDR3, VL CDR1, VL CDR2, and VL CDR3 amino acid sequences of the first binding domain are according to the Exemplary numbering system.

In some embodiments, the first binding domain comprises a VH comprising a VH CDR1 having an amino acid sequence of SEQ ID NO:1, a VH CDR2 having an amino acid sequence of SEQ ID NO:2, and a VH CDR3 having an amino acid sequence of SEQ ID NO:3. In some embodiments, the first binding domain comprises a VL comprising a VL CDR1 having an amino acid sequence of SEQ ID NO:4, a VL CDR2 having an amino acid sequence of SEQ ID NO:5, and a VL CDR3 having an amino acid sequence of SEQ ID NO:6. In some embodiments, the first binding domain comprises: (i) a VH comprising a VH CDR1 having an amino acid sequence of SEQ ID NO: 1, a VH CDR2 having an amino acid sequence of SEQ ID NO:2, and a VH CDR3 having an amino acid sequence of SEQ ID NO:3; and (ii) a VL comprising a VL CDR1 having an amino acid sequence of SEQ ID NO: 4, a VL CDR2 having an amino acid sequence of SEQ ID NO:5, and a VL CDR3 having an amino acid sequence of SEQ ID NO:6.

In some embodiments, the first binding domain comprises a VH having an amino acid sequence of SEQ ID NO:7. In some embodiments, the first binding domain comprises a VL having an amino acid sequence of SEQ ID NO:8. In some embodiments, the first binding domain comprises a VH having an amino acid sequence of SEQ ID NO:7, and a VL having an amino acid sequence of SEQ ID NO: 8.

In some embodiments, the first binding domain specifically binds to the TRDV2. In some embodiments, the first binding domain binds a TRDV2 antigen. In some embodiments, the first binding domain binds a TRDV2 epitope. In some embodiments, the VH CDR1, VH CDR2, VH CDR3, VL CDR1, VL CDR2 and VL CDR3 of the first binding domain form a binding site for an antigen of the TRDV2. In some embodiments, the VH CDR1, VH CDR2, VH CDR3, VL CDR1, VL CDR2 and VL CDR3 of the first binding domain form a binding site for an epitope of the TRDV2.

In some embodiments, the TRDV2 is present on the surface of a T cell.

In some embodiments, the cancer cell is a cell of an adrenal cancer, anal cancer, appendix cancer, bile duct cancer, bladder cancer, bone cancer, brain cancer, breast cancer, cervical cancer, colorectal cancer, esophageal cancer, gallbladder cancer, gestational trophoblastic, head and neck cancer, Hodgkin lymphoma, intestinal cancer, kidney cancer, leukemia, liver cancer, lung cancer, melanoma, mesothelioma, multiple myeloma, neuroendocrine tumor, non-Hodgkin lymphoma, oral cancer, ovarian cancer, pancreatic cancer, prostate cancer, sinus cancer, skin cancer, soft tissue sarcoma spinal cancer, stomach cancer, testicular cancer, throat cancer, thyroid cancer, uterine cancer endometrial cancer, vaginal cancer, or vulvar cancer.

In some embodiments, the antigen on the surface of the cancer cell is angiopoietin, BCMA, CD19, CD20, CD22, CD25 (IL2-R), CD30, CD33, CD37, CD38, CD52, CD56, CD123 (IL-3R), cMET, DLL/Notch, EGFR, EpCAM, FGF, FGF-R, GD2, HER2, Mesothelin, Nectin-4, PAP, PDGFRα, PSA, PSA3, PSMA, RANKL, SLAMF7, STEAP1, TARP, TROP2, VEGF, or VEGF-R.

In some embodiments, the antigen on the surface of the cancer cell is CEA, immature laminin receptor, TAG-72, HPV E6, HPV E7, BING-4, calcium-activated chloride channel 2, cyclin-B1, 9D7, EpCAM, EphA3, Her2/neu, telomerase, mesothelin, SAP-1, surviving, a BAGE family antigen, CAGE family antigen, GAGE family antigen, MAGE family antigen, SAGE family antigen, XAGE family antigen, NY-ESO-1/LAGE-1, PRAME, SSX-2, Melan-A, MART-1, Gp100, pmel17, tyrosinase, TRP-1, TRP-2, P. polypeptide, MCIR, prostate-specific antigen, β-catenin, or BRCA1.

In some embodiments, the antigen on the surface of the cancer cell is CD33.

In some embodiments, the second binding domain comprises a VH comprising a VH CDR1, a VH CDR2, and a VH CDR3 having an amino acid sequence of a VH CDR1, a VH CDR2, and a VH CDR3, respectively, of a VH having an amino acid sequence of SEQ ID NO: 15. In some embodiments, the second binding domain comprises a VL comprising a VL CDR1, a VL CDR2, and a VL CDR3 having an amino acid sequence of a VL CDR1, a VL CDR2, and a VL CDR3, respectively, of a VL having an amino acid sequence of SEQ ID NO: 16. In some embodiments, the second binding domain comprises: (i) a VH comprising a VH CDR1, a VH CDR2, and a VH CDR3 having an amino acid sequence of a VH CDR1, a VH CDR2, and a VH CDR3, respectively, of a VH having an amino acid sequence of SEQ ID NO: 15; and (ii) a VL comprising a VL CDR1, a VL CDR2, and a VL CDR3 having an amino acid sequence of a VL CDR1, a VL CDR2, and a VL CDR3, respectively, of a VL having an amino acid sequence of SEQ ID NO: 16. In some embodiments, the VH CDR1, VH CDR2, VH CDR3, VL CDR1, VL CDR2, and VL CDR3 amino acid sequences of the second binding domain are according to the Kabat numbering system. In some embodiments, the VH CDR1, VH CDR2, VH CDR3, VL CDR1, VL CDR2, and VL CDR3 amino acid sequences of the second binding domain are according to the Chothia numbering system. In some embodiments, the VH CDR1, VH CDR2, VH CDR3, VL CDR1, VL CDR2, and VL CDR3 amino acid sequences of the second binding domain are according to the AbM numbering system. In some embodiments, the VH CDR1, VH CDR2, VH CDR3, VL CDR1, VL CDR2, and VL CDR3 amino acid sequences of the second binding domain are according to the Contact numbering system. In some embodiments, the VH CDR1, VH CDR2, VH CDR3, VL CDR1, VL CDR2, and VL CDR3 amino acid sequences of the second binding domain are according to the IMGT numbering system. In some embodiments, the VH CDR1, VH CDR2, VH CDR3, VL CDR1, VL CDR2, and VL CDR3 amino acid sequences of the second binding domain are according to the Exemplary numbering system.

In some embodiments, the second binding domain comprises a VH comprising a VH CDR1 having an amino acid sequence of SEQ ID NO:9, a VH CDR2 having an amino acid sequence of SEQ ID NO:10, and a VH CDR3 having an amino acid sequence of SEQ ID NO:11. In some embodiments, the second binding domain comprises a VL comprising a VL CDR1 having an amino acid sequence of SEQ ID NO:12, a VL CDR2 having an amino acid sequence of SEQ ID NO:13, and a VL CDR3 having an amino acid sequence of SEQ ID NO: 14. In some embodiments, the second binding domain comprises: (i) a VH comprising a VH CDR1 having an amino acid sequence of SEQ ID NO:9, a VH CDR2 having an amino acid sequence of SEQ ID NO: 10, and a VH CDR3 having an amino acid sequence of SEQ ID NO: 11; and (ii) a VL comprising a VL CDR1 having an amino acid sequence of SEQ ID NO: 12, a VL CDR2 having an amino acid sequence of SEQ ID NO:13, and a VL CDR3 having an amino acid sequence of SEQ ID NO: 14.

In some embodiments, the second binding domain comprises a VH having an amino acid sequence of SEQ ID NO: 15. In some embodiments, the second binding domain comprises a VL having an amino acid sequence of SEQ ID NO:16. In some embodiments, the second binding domain comprises a VH having an amino acid sequence of SEQ ID NO: 15, and a VL having an amino acid sequence of SEQ ID NO: 16.

In some embodiments, the VH CDR1, VH CDR2, VH CDR3, VL CDR1, VL CDR2 and VL CDR3 of the second binding domain form a binding site for an antigen of the CD33. In some embodiments, the VH CDR1, VH CDR2, VH CDR3, VL CDR1, VL CDR2 and VL CDR3 of the first binding domain form an epitope for the CD33.

In some embodiments, the second binding domain specifically binds to the antigen on the surface of the cancer cell.

In some embodiments, the first binding domain is multivalent, the second binding domain is multivalent, or wherein both the first binding domain and the second binding domain are multivalent. In some embodiments, the first binding domain is multivalent. In some embodiments, the second binding domain is multivalent. In some embodiments, both the first binding domain and the second binding domain are multivalent.

In some embodiments, the first binding domain is capable of binding at least two antigens, or wherein the second binding domain is capable of binding at least two antigens. In some embodiments, the first binding domain is capable of binding at least two antigens. In some embodiments, the second binding domain is capable of binding at least two antigens. In some embodiments, the first binding domain is capable of binding at least three antigens, or wherein the second binding domain is capable of binding at least three antigens. In some embodiments, the first binding domain is capable of binding at least three antigens. In some embodiments, the second binding domain is capable of binding at least three antigens. In some embodiments, the first binding domain is capable of binding at least four antigens, or wherein the second binding domain is capable of binding at least four antigens. In some embodiments, the first binding domain is capable of binding at least four antigens. In some embodiments, the second binding domain is capable of binding at least four antigens. In some embodiments, the first binding domain is capable of binding at least five antigens, or wherein the second binding domain is capable of binding at least five antigens. In some embodiments, the first binding domain is capable of binding at least five antigens. In some embodiments, the second binding domain is capable of binding at least five antigens.

In some embodiments, the multispecific antibody is a bispecific antibody. In some embodiments, the multispecific antibody is a trispecific antibody. In some embodiments, the multispecific antibody is a quadraspecific antibody.

In some embodiments, the antibody is a humanized antibody. In some embodiments, the antibody is an IgG antibody. In some embodiments, the IgG antibody is an IgG1, IgG2, IgG3, or lgG4 antibody. In some embodiments, the antibody comprises a kappa light chain. In some embodiments, the antibody comprises a lambda light chain. In some embodiments, the antibody is a monoclonal antibody.

In another aspect, provided is a multispecific antibody comprising: a first means capable of binding TRDV2 on the surface of a T cell; and a second means capable of binding a cancer antigen. In certain embodiments, the T cell is a γδ T cell. In some embodiments, the cancer antigen is on the surface of a cancer cell. In some embodiments, the multispecific antibody is a bispecific antibody.

In another general aspect, provided herein is an isolated nucleic acid encoding a monoclonal antibody or antigen-binding fragment thereof provided herein. In another general aspect, provided herein is an isolated nucleic acid encoding a bispecific antibody or antigen-binding fragment thereof. It will be appreciated by those skilled in the art that the coding sequence of a protein can be changed (e.g., replaced, deleted, inserted, etc.) without changing the amino acid sequence of the protein. Accordingly, it will be understood by those skilled in the art that nucleic acid sequences encoding monoclonal antibodies and/or bispecific antibodies provided herein can be altered without changing the amino acid sequences of the proteins.

In another general aspect, provided herein is a vector comprising an isolated nucleic acid encoding a monoclonal antibody or antigen-binding fragment thereof. In another general aspect, provided herein is a vector comprising an isolated nucleic acid encoding a bispecific antibody or antigen-binding fragment thereof. Any vector known to those skilled in the art in view of the present disclosure can be used, such as a plasmid, a cosmid, a phage vector or a viral vector. In some embodiments, the vector is a recombinant expression vector such as a plasmid. The vector can include any element to establish a conventional function of an expression vector, for example, a promoter, ribosome binding element, terminator, enhancer, selection marker, and origin of replication. The promoter can be a constitutive, inducible or repressible promoter. A number of expression vectors capable of delivering nucleic acids to a cell are known in the art and can be used herein for production of an antibody or antigen-binding fragment thereof in the cell. Conventional cloning techniques or artificial gene synthesis can be used to generate a recombinant expression vector according to embodiments provided herein. Such techniques are well known to those skilled in the art in view of the present disclosure.

In another general aspect, provided herein is a host cell comprising an isolated nucleic acid encoding a monoclonal antibody and/or bispecific antibody or an antigen-binding fragment provided herein. Any host cell known to those skilled in the art in view of the present disclosure can be used for recombinant expression of antibodies or antigen-binding fragments thereof provided herein. In some embodiments, the host cells are E. coli TG1 or BL21 cells (for expression of, e.g., an scFv or Fab antibody), CHO-DG44 or CHO-K1 cells or HEK293 cells (for expression of, e.g., a full-length IgG antibody). According to particular embodiments, the recombinant expression vector is transformed into host cells by conventional methods such as chemical transfection, heat shock, or electroporation, where it is stably integrated into the host cell genome such that the recombinant nucleic acid is effectively expressed.

In another aspect, provided is a nucleic acid encoding a multispecific TRDV2 antibody provided herein. In another aspect, provided is a vector comprising the nucleic acid encoding a multispecific TRDV2 antibody provided herein. In another aspect, provided is a host cell comprising a vector comprising the nucleic acid encoding a multispecific TRDV2 antibody provided herein. In another aspect, provided is a kit comprising a vector comprising the nucleic acid encoding a multispecific TRDV2 antibody provided herein, and packaging for the same

In another aspect, provided is a pharmaceutical composition comprising a multispecific TRDV2 antibody provided herein, and a pharmaceutically acceptable carrier.

In another aspect, provided is a method of producing a pharmaceutical composition comprising a multispecific TRDV2 antibody provided herein, comprising combining the multispecific antibody with a pharmaceutically acceptable carrier to obtain the pharmaceutical composition.

In another aspect, provided is a process for making an antibody that binds to more than one target molecule, the molecule comprising: a step for performing a function of obtaining a binding domain capable of binding to TRDV2 antigen on a γδ T cell; a step for performing a function of obtaining a binding domain capable of binding to an antigen on the surface of a cancer cell; and a step for performing a function of providing an antibody capable of binding to a TRDV2 antigen on a γδ T cell and an antigen on the surface of a cancer cell. In some embodiments, the step for performing a function of obtaining a binding domain capable of binding to an antigen on the surface of a cancer cell is repeated n times and further comprising n steps for performing a function of providing a binding domain capable of binding to a TRDV2 antigen on a γδ T cell and n number of target molecules, wherein n is at least 2.

In another general aspect, provided herein is a method of producing a bispecific antibody or antigen-binding fragment thereof disclosed herein. The methods comprise culturing a cell comprising a nucleic acid encoding the bispecific antibody or antigen-binding fragment thereof under conditions to produce a bispecific antibody or antigen-binding fragment thereof disclosed herein and recovering the antibody or antigen-binding fragment thereof from the cell or cell culture (e.g., from the supernatant). Expressed antibodies or antigen-binding fragments thereof can be harvested from the cells and purified according to conventional techniques known in the art and as described herein.

### Pharmaceutical Compositions

In another general aspect, provided herein is a pharmaceutical composition comprising an isolated bispecific antibody or antigen-binding fragment thereof and a pharmaceutically acceptable carrier. The term “pharmaceutical composition” as used herein means a product comprising an antibody provided herein together with a pharmaceutically acceptable carrier. Antibodies provided herein, and compositions comprising the antibodies, are also useful in the manufacture of a medicament for therapeutic applications mentioned herein.

As used herein, the term “carrier” refers to any excipient, diluent, filler, salt, buffer, stabilizer, solubilizer, oil, lipid, lipid containing vesicle, microsphere, liposomal encapsulation, or other material well known in the art for use in pharmaceutical formulations. It will be understood that the characteristics of the carrier, excipient or diluent will depend on the route of administration for a particular application. As used herein, the term “pharmaceutically acceptable carrier” refers to a non-toxic material that does not interfere with the effectiveness of a composition provided herein or the biological activity of a composition provided herein. According to particular embodiments, in view of the present disclosure, any pharmaceutically acceptable carrier suitable for use in an antibody pharmaceutical composition can be used herein.

The formulation of pharmaceutically active ingredients with pharmaceutically acceptable carriers is known in the art, e.g., Remington: The Science and Practice of Pharmacy (e.g. 21st edition (2005), and any later editions). Non-limiting examples of additional ingredients include: buffers, diluents, solvents, tonicity regulating agents, preservatives, stabilizers, and chelating agents. One or more pharmaceutically acceptable carriers can be used in formulating the pharmaceutical compositions provided herein.

In one embodiment, the pharmaceutical composition is a liquid formulation. An exemplary liquid formulation is an aqueous formulation, i.e., a formulation comprising water. The liquid formulation can comprise a solution, a suspension, an emulsion, a microemulsion, a gel, and the like. An aqueous formulation typically comprises at least 50% w/w water, or at least 60%, 70%, 75%, 80%, 85%, 90%, or at least 95% w/w of water.

In one embodiment, the pharmaceutical composition can be formulated as an injectable which can be injected, for example, via an injection device (e.g., a syringe or an infusion pump). The injection can be delivered subcutaneously, intramuscularly, intraperitoneally, intravitreally, or intravenously, for example.

In another embodiment, the pharmaceutical composition is a solid formulation, e.g., a freeze-dried or spray-dried composition, which can be used as is, or whereto the physician or the patient adds solvents, and/or diluents prior to use. Solid dosage forms can include tablets, such as compressed tablets, and/or coated tablets, and capsules (e.g., hard or soft gelatin capsules). The pharmaceutical composition can also be in the form of sachets, dragees, powders, granules, lozenges, or powders for reconstitution, for example.

The dosage forms can be immediate release, in which case they can comprise a water-soluble or dispersible carrier, or they can be delayed release, sustained release, or modified release, in which case they can comprise water-insoluble polymers that regulate the rate of dissolution of the dosage form in the gastrointestinal tract or under the skin.

In other embodiments, the pharmaceutical composition can be delivered intranasally, intrabuccally, or sublingually.

The pH in an aqueous formulation can be between pH 3 and pH 10. In one embodiment, the pH of the formulation is from about 7.0 to about 9.5. In another embodiment, the pH of the formulation is from about 3.0 to about 7.0.

In another embodiment, the pharmaceutical composition comprises a buffer. Non-limiting examples of buffers include: arginine, aspartic acid, bicine, citrate, disodium hydrogen phosphate, fumaric acid, glycine, glycylglycine, histidine, lysine, maleic acid, malic acid, sodium acetate, sodium carbonate, sodium dihydrogen phosphate, sodium phosphate, succinate, tartaric acid, tricine, and tris(hydroxymethyl)-aminomethane, and mixtures thereof. The buffer can be present individually or in the aggregate, in a concentration from about 0.01 mg/ml to about 50 mg/ml, for example from about 0.1 mg/ml to about 20 mg/ml. Pharmaceutical compositions comprising each one of these specific buffers constitute alternative embodiments.

In another embodiment, the pharmaceutical composition comprises a preservative. Non-limiting examples of preservatives include: benzethonium chloride, benzoic acid, benzyl alcohol, bronopol, butyl 4-hydroxybenzoate, chlorobutanol, chlorocresol, chlorohexidine, chlorphenesin, o-cresol, m-cresol, p-cresol, ethyl 4-hydroxybenzoate, imidurea, methyl 4-hydroxybenzoate, phenol, 2-phenoxyethanol, 2-phenylethanol, propyl 4-hydroxybenzoate, sodium dehydroacetate, thiomerosal, and mixtures thereof. The preservative can be present individually or in the aggregate, in a concentration from about 0.01 mg/ml to about 50 mg/ml, for example from about 0.1 mg/ml to about 20 mg/ml. Pharmaceutical compositions comprising each one of these specific preservatives constitute alternative embodiments.

In another embodiment, the pharmaceutical composition comprises an isotonic agent. Non-limiting examples of isotonic agents include a salt (such as sodium chloride), an amino acid (such as glycine, histidine, arginine, lysine, isoleucine, aspartic acid, tryptophan, and threonine), an alditol (such as glycerol, 1,2-propanediol propyleneglycol), 1,3-propanediol, and 1,3-butanediol), polyethyleneglycol (e.g. PEG400), and mixtures thereof. Another example of an isotonic agent includes a sugar. Non-limiting examples of sugars can include mono-, di-, or polysaccharides, or water-soluble glucans, including for example fructose, glucose, mannose, sorbose, xylose, maltose, lactose, sucrose, trehalose, dextran, pullulan, dextrin, cyclodextrin, alpha and beta-HPCD, soluble starch, hydroxyethyl starch, and sodium carboxymethyl-cellulose. Another example of an isotonic agent is a sugar alcohol, wherein the term “sugar alcohol” is defined as a C (4-8) hydrocarbon having at least one-OH group. Non-limiting examples of sugar alcohols include mannitol, sorbitol, inositol, galactitol, dulcitol, xylitol, and arabitol. The isotonic agent can be present individually or in the aggregate, in a concentration from about 0.01 mg/ml to about 50 mg/ml, for example from about 0.1 mg/ml to about 20 mg/ml. Pharmaceutical compositions comprising each one of these specific isotonic agents constitute alternative embodiments.

In another embodiment, the pharmaceutical composition comprises a chelating agent. Non-limiting examples of chelating agents include citric acid, aspartic acid, salts of ethylenediaminetetraacetic acid (EDTA), and mixtures thereof. The chelating agent can be present individually or in the aggregate, in a concentration from about 0.01 mg/ml to about 50 mg/ml, for example from about 0.1 mg/ml to about 20 mg/ml. Pharmaceutical compositions comprising each one of these specific chelating agents constitute alternative embodiments.

In another embodiment, the pharmaceutical composition comprises a stabilizer. Non-limiting examples of stabilizers include one or more aggregation inhibitors, one or more oxidation inhibitors, one or more surfactants, and/or one or more protease inhibitors.

In another embodiment, the pharmaceutical composition comprises a stabilizer, wherein said stabilizer is carboxy-/hydroxycellulose and derivates thereof (such as HPC, HPC-SL, HPC-L and HPMC), cyclodextrins, 2-methylthioethanol, polyethylene glycol (such as PEG 3350), polyvinyl alcohol (PVA), polyvinyl pyrrolidone, salts (such as sodium chloride), sulphur-containing substances such as monothioglycerol), or thioglycolic acid. The stabilizer can be present individually or in the aggregate, in a concentration from about 0.01 mg/ml to about 50 mg/ml, for example from about 0.1 mg/ml to about 20 mg/ml. Pharmaceutical compositions comprising each one of these specific stabilizers constitute alternative embodiments.

In further embodiments, the pharmaceutical composition comprises one or more surfactants, such as a surfactant, at least one surfactant, or two different surfactants. The term “surfactant” refers to any molecules or ions that are comprised of a water-soluble (hydrophilic) part, and a fat-soluble (lipophilic) part. The surfactant can, for example, be selected from the group consisting of anionic surfactants, cationic surfactants, nonionic surfactants, and/or zwitterionic surfactants. The surfactant can be present individually or in the aggregate, in a concentration from about 0.1 mg/ml to about 20 mg/ml. Pharmaceutical compositions comprising each one of these specific surfactants constitute alternative embodiments.

In a further embodiment, the pharmaceutical composition comprises one or more protease inhibitors, such as, e.g., EDTA, and/or benzamidine hydrochloric acid (HC1). The protease inhibitor can be present individually or in the aggregate, in a concentration from about 0.1 mg/ml to about 20 mg/ml. Pharmaceutical compositions comprising each one of these specific protease inhibitors constitute alternative embodiments.

In another general aspect, provided herein is a method of producing a pharmaceutical composition comprising a bispecific antibody or antigen-binding fragment thereof provided herein, comprising combining a bispecific antibody or antigen-binding fragment thereof with a pharmaceutically acceptable carrier to obtain the pharmaceutical composition.

### Alternative Binding Agents

While TRDV2 antibodies are exemplified herein, it is understood that other molecules that bind to TRDV2 (TRDV2 molecules) are also contemplated. Such TRDV2 molecules include alternative binding agents, include equivalents of the antibodies provided herein. In some embodiments, the TRDV2 molecules of the present disclosure comprise a non-immunoglobulin binding agent. In some embodiments, the first binding domain comprises a non-immunoglobulin binding agent. In some embodiments, the second binding domain comprises a non-immunoglobulin binding agent.

In certain embodiments, such a non-immunoglobulin binding agent can bind the same targets exemplified herein. For example, in som embodiment, the non-immunoglobulin binding agent can bind to the same epitope as an antibody disclosed herein. In some embodiments, a non-immunoglobulin binding agent is identified as an agent that displaces or is displaced by an antibody of the present disclosure in a competitive binding assay. These alternative binding agents may include, for example, any of the engineered protein scaffolds known in the art. Such scaffolds include, for example, anticalins, which are based upon the lipocalin scaffold, a protein structure characterized by a rigid beta-barrel that supports four hypervariable loops which form the ligand binding site. Novel binding specificities may be engineered by targeted random mutagenesis in the loop regions, in combination with functional display and guided selection (see, e.g., Skerra, 2008, FEBS J. 275:2677-83). Other suitable scaffolds may include, for example, adnectins, or monobodies, based on the tenth extracellular domain of human fibronectin III (see, e.g., Koide and Koide, 2007, Methods Mol. Biol. 352:95-109); affibodies, based on the Z domain of staphylococcal protein A (see, e.g., Nygren et al., 2008, FEBS J. 275:2668-76); DARPins, based on ankyrin repeat proteins (see, e.g., Stumpp et al., 2008, Drug. Discov. Today 13:695-701); fynomers, based on the SH3 domain of the human Fyn protein kinase (see, e.g., Grabulovski et al., 2007, J. Biol. Chem. 282:3196-204); affitins, based on Sac7d from Sulfolobus acidolarius (see, e.g., Krehenbrink et al., 2008, J. Mol. Biol. 383:1058-68); affilins, based on human y-B-crystallin (see, e.g., Ebersbach et al., 2007, J. Mol. Biol. 372:172-85); avimers, based on the A domain of membrane receptor proteins (see, e.g., Silverman et al., 2005, Biotechnol. 23:1556-61); cysteine-rich knottin peptides (see, e.g., Kolmar, 2008, FEBS J. 275:2684-90); and engineered Kunitz-type inhibitors (see, e.g., Nixon and Wood, 2006, Curr. Opin. Drug. Discov. Dev. 9:261-68). For a review, see, for example, Gebauer and Skerra, 2009, Curr. Opin. Chem. Biol. 13:245-55.

### Methods of Use

Also provided is a method of targeting an antigen on the surface of a cancer cell, the method comprising exposing the cancer cell to an anti-TRDV2 bispecific antibody or antigen binding fragment thereof provided herein. Also provided is a method of targeting an antigen on the surface of a cancer cell, the method comprising exposing the cancer cell to a pharmaceutical composition comprising an anti-TRDV2 bispecific antibody or antigen binding fragment thereof provided herein

The functional activity of bispecific antibodies and antigen-binding fragments thereof that bind TRDV2 or a cancer antigen can be characterized by methods known in the art and as described herein. Methods for characterizing antibodies and antigen-binding fragments thereof that bind TRDV2 or a cancer antigen include, but are not limited to, affinity and specificity assays including Biacore, ELISA, and OCTETRED analysis; binding assays to detect the binding of antibodies to cancer cells by FACS; binding assays to detect the binding of antibodies to TRDV2 on γδ T cells. According to particular embodiments, the methods for characterizing antibodies and antigen-binding fragments thereof that bind TRDV2 or a cancer antigen include those described below.

In one aspect, provided is a method of directing a yd T cell expressing TRDV2 to a cancer cell, the method comprising contacting the γδ T cell with a multispecific TRDV2 antibody provided herein, wherein the contacting directs the yo T cell to the cancer cell. In another aspect, provided is a method of inhibiting growth or proliferation of cancer cells expressing a cancer antigen on the cell surface, the method comprising contacting the cancer cells with a multispecific TRDV2 antibody provided herein, wherein contacting the cancer cells with the pharmaceutical composition inhibits growth or proliferation of the cancer cells. In some embodiments, the cancer cells are in the presence of a γδ T cell expressing TRDV2 while in contact with the multispecific antibody.

In one aspect, provided is a method for eliminating cancer cells in a subject, comprising administering an effective amount of a multispecific TRDV2 antibody provided herein to the subject. In another aspect, provided is a method for treating cancer in a subject, comprising administering an effective amount of a multispecific TRDV2 antibody provided herein to the subject. In some embodiments, the subject is a subject in need thereof. In some embodiments, the subject is a human.

In one aspect, provided is a method of activating a γδ T cell expressing TRDV2, comprising contacting the γδ T cell with the multispecific TRDV2 antibody provided herein. In some embodiments, the contacting results in an increase in CD69, CD25, and/or Granzyme B expression, as compared to a control γδ T cell expressing TRDV2.

Also provided is a method of directing Vδ2-expressing γδ T cells to a cancer cell. The methods can comprise contacting the Vδ2-expressing γδ T cell with an anti-TRDV2 bispecific antibody or antigen binding fragment thereof provided herein, wherein the anti-TRDV2 bispecific antibody or antigen binding fragment thereof directs the Vδ2-expressing γδ T cell to the cancer. Also provided is a method of directing a γδ T cell expressing TRDV2 to a cancer cell, the method comprising contacting the γδ T cell with a bispecific antibody provided herein, wherein the contacting directs the γδ T cell to the cancer cell.

Also provided is a method for inhibiting growth or proliferation of cancer cells. The methods can comprise contacting the Vδ2-expressing γδ T cells with an anti-TRDV2 bispecific antibody or antigen binding fragment thereof provided herein, wherein contacting the cancer cells with the anti-TRDV2 bispecific antibody or antigen binding fragment thereof composition inhibits the growth or proliferation of the cancer cells. Also provided is a method of inhibiting growth or proliferation of cancer cells expressing a cancer antigen on the cell surface, the method comprising contacting the cancer cells with a bispecific antibody provided herein, wherein contacting the cancer cells with the pharmaceutical composition inhibits growth or proliferation of the cancer cells. In some embodiments, the cancer cells are in the presence of a γδ T cell expressing TRDV2 while in contact with the bispecific antibody.

In another general aspect, provided herein is a method of treating a cancer in a subject in need thereof, comprising administering to the subject an isolated bispecific antibody or antigen binding fragment thereof that specifically binds TRDV2 and a cancer antigen presented on the surface of a cancer cell, or a pharmaceutical composition disclosed herein. In some embodiments, provided is a method for eliminating cancer cells expressing the cancer antigen in a subject, comprising administering an effective amount of a bispecific antibody provided herein to the subject. In some embodiments, provided is a method for treating a disease caused all or in part by cancer cells expressing the cancer antigen in a subject, comprising administering an effective amount of a bispecific antibody provided herein to the subject. In some embodiments, the subject is a subject in need thereof. In some embodiments, the subject is a human. In some embodiments, the disease is cancer. In specific embodiments, the bispecific antibody binds TRDV2 and a cancer antigen. In a certain embodiments, the cancer is a CD33-expressing cancer.

In some embodiments, the antigen on the surface of the cancer cell is a tumor-specific antigen, a tumor-associated antigen, or a neoantigen.

In some embodiments, the cancer cell is a cell of an adrenal cancer, anal cancer, appendix cancer, bile duct cancer, bladder cancer, bone cancer, brain cancer, breast cancer, cervical cancer, colorectal cancer, esophageal cancer, gallbladder cancer, gestational trophoblastic, head and neck cancer, Hodgkin lymphoma, intestinal cancer, kidney cancer, leukemia, liver cancer, lung cancer, melanoma, mesothelioma, multiple myeloma, neuroendocrine tumor, non-Hodgkin lymphoma, oral cancer, ovarian cancer, pancreatic cancer, prostate cancer, sinus cancer, skin cancer, soft tissue sarcoma spinal cancer, stomach cancer, testicular cancer, throat cancer, thyroid cancer, uterine cancer endometrial cancer, vaginal cancer, or vulvar cancer.

In some embodiments, the adrenal cancer is an adrenocortical carcinoma (ACC), adrenal cortex cancer, pheochromocytoma, or neuroblastoma.

In some embodiments, the anal cancer is a squamous cell carcinoma, cloacogenic carcinoma, adenocarcinoma, basal cell carcinoma, or melanoma.

In some embodiments, the appendix cancer is a neuroendocrine tumor (NET), mucinous adenocarcinoma, goblet cell carcinoid, intestinal-type adenocarcinoma, or signet-ring cell adenocarcinoma.

In some embodiments, the bile duct cancer is an extrahepatic bile duct cancer, adenocarcinomas, hilar bile duct cancer, perihilar bile duct cancer, distal bile duct cancer, or intrahepatic bile duct cancer.

In some embodiments, the bladder cancer is transitional cell carcinoma (TCC), papillary carcinoma, flat carcinoma, squamous cell carcinoma, adenocarcinoma, small-cell carcinoma, or sarcoma.

In some embodiments, the bone cancer is a primary bone cancer, sarcoma, osteosarcoma, chondrosarcoma, sarcoma, fibrosarcoma, malignant fibrous histiocytoma, giant cell tumor of bone, chordoma, or metastatic bone cancer.

In some embodiments, the brain cancer is an astrocytoma, brain stem glioma, glioblastoma, meningioma, ependymoma, oligodendroglioma, mixed glioma, pituitary carcinoma, pituitary adenoma, craniopharyngioma, germ cell tumor, pineal region tumor, medulloblastoma, or primary CNS lymphoma.

In some embodiments, the breast cancer is a breast adenocarcinoma, invasive breast cancer, noninvasive breast cancer, breast sarcoma, metaplastic carcinoma, adenocystic carcinoma, phyllodes tumor, angiosarcoma, HER2-positive breast cancer, triple-negative breast cancer, or inflammatory breast cancer.

In some embodiments, the cervical cancer is a squamous cell carcinoma, or adenocarcinoma.

In some embodiments, the colorectal cancer is a colorectal adenocarcinoma, primary colorectal lymphoma, gastrointestinal stromal tumor, leiomyosarcoma, carcinoid tumor, mucinous adenocarcinoma, signet ring cell adenocarcinoma, gastrointestinal carcinoid tumor, or melanoma.

In some embodiments, the esophageal cancer is an adenocarcinoma or squamous cell carcinoma.

In some embodiments, the gall bladder cancer is an adenocarcinoma, papillary adenocarcinoma, adenosquamous carcinoma, squamous cell carcinoma, small cell carcinoma, or sarcoma.

In some embodiments, the gestational trophoblastic disease (GTD) is a hydatidiform mole, gestational trophoblastic neoplasia (GTN), choriocarcinoma, placental-site trophoblastic tumor (PSTT), or epithelioid trophoblastic tumor (ETT).

In some embodiments, the head and neck cancer is a laryngeal cancer, nasopharyngeal cancer, hypopharyngeal cancer, nasal cavity cancer, paranasal sinus cancer, salivary gland cancer, oral cancer, oropharyngeal cancer, or tonsil cancer.

In some embodiments, the Hodgkin lymphoma is a classical Hodgkin lymphoma, nodular sclerosis, mixed cellularity, lymphocyte-rich, lymphocyte-depleted, or nodular lymphocyte-predominant Hodgkin lymphoma (NLPHL).

In some embodiments, the intestinal cancer is a small intestine cancer, small bowel cancer, adenocarcinoma, sarcoma, gastrointestinal stromal tumors, carcinoid tumors, or lymphoma.

In some embodiments, the kidney cancer is a renal cell carcinoma (RCC), clear cell RCC, papillary RCC, chromophobe RCC, collecting duct RCC, unclassified RCC, transitional cell carcinoma, urothelial cancer, renal pelvis carcinoma, or renal sarcoma.

In some embodiments, the leukemia is an acute lymphocytic leukemia (ALL), acute myeloid leukemia (AML), chronic lymphocytic leukemia (CLL), chronic myeloid leukemia (CML), hairy cell leukemia (HCL), or a myelodysplastic syndrome (MDS).

In some embodiments, the liver cancer is a hepatocellular carcinoma (HCC), fibrolamellar HCC, cholangiocarcinoma, angiosarcoma, or liver metastasis.

In some embodiments, the lung cancer is a small cell lung cancer, small cell carcinoma, combined small cell carcinoma, non-small cell lung cancer, lung adenocarcinoma, squamous cell lung cancer, large-cell undifferentiated carcinoma, pulmonary nodule, metastatic lung cancer, adenosquamous carcinoma, large cell neuroendocrine carcinoma, salivary gland-type lung carcinoma, lung carcinoid, mesothelioma, sarcomatoid carcinoma of the lung, or malignant granular cell lung tumor.

In some embodiments, the melanoma is a superficial spreading melanoma, nodular melanoma, acral-lentiginous melanoma, lentigo maligna melanoma, amelanotic melanoma, desmoplastic melanoma, ocular melanoma, or metastatic melanoma.

In some embodiments, the mesothelioma is a pleural mesothelioma, peritoneal mesothelioma, pericardial mesothelioma, or testicular mesothelioma.

In some embodiments, the multiple myeloma is an active myeloma or smoldering myeloma.

In some embodiments, the neuroendocrine tumor, is a gastrointestinal neuroendocrine tumor, pancreatic neuroendocrine tumor, or lung neuroendocrine tumor.

In some embodiments, the non-Hodgkin's lymphoma is an anaplastic large-cell lymphoma, lymphoblastic lymphoma, peripheral T cell lymphoma, follicular lymphoma, cutaneous T cell lymphoma, lymphoplasmacytic lymphoma, marginal zone B-cell lymphoma, MALT lymphoma, small-cell lymphocytic lymphoma, Burkitt lymphoma, chronic lymphocytic leukemia (CLL), small lymphocytic lymphoma (SLL), precursor T-lymphoblastic leukemia/lymphoma, acute lymphocytic leukemia (ALL), adult T cell lymphoma/leukemia (ATLL), hairy cell leukemia, B-cell lymphomas, diffuse large B-cell lymphoma (DLBCL), primary mediastinal B-cell lymphoma, primary central nervous system (CNS) lymphoma, mantle cell lymphoma (MCL), marginal zone lymphomas, mucosa-associated lymphoid tissue (MALT) lymphoma, nodal marginal zone B-cell lymphoma, splenic marginal zone B-cell lymphoma, lymphoplasmacytic lymphoma, B-cell non-Hodgkin lymphoma, T cell non-Hodgkin lymphoma, natural killer cell lymphoma, cutaneous T cell lymphoma, Alibert-Bazin syndrome, Sezary syndrome, primary cutaneous anaplastic large-cell lymphoma, peripheral T cell lymphoma, angioimmunoblastic T cell lymphoma (AITL), anaplastic large-cell lymphoma (ALCL), systemic ALCL, enteropathy-type T cell lymphoma (EATL), or hepatosplenic gamma/delta T cell lymphoma.

In some embodiments, the oral cancer is a squamous cell carcinoma, verrucous carcinoma, minor salivary gland carcinomas, lymphoma, benign oral cavity tumor, cosinophilic granuloma, fibroma, granular cell tumor, karatoacanthoma, leiomyoma, ostcochondroma, lipoma, schwannoma, neurofibroma, papilloma, condyloma acuminatum, verruciform xanthoma, pyogenic granuloma, rhabdomyoma, odontogenic tumors, leukoplakia, crythroplakia, squamous cell lip cancer, basal cell lip cancer, mouth cancer, gum cancer, or tongue cancer.

In some embodiments, the ovarian cancer is a ovarian epithelial cancer, mucinous epithelial ovarian cancer, endometrioid epithelial ovarian cancer, clear cell epithelial ovarian cancer, undifferentiated epithelial ovarian cancer, ovarian low malignant potential tumors, primary peritoneal carcinoma, fallopian tube cancer, germ cell tumors, teratoma, dysgerminoma ovarian germ cell cancer, endodermal sinus tumor, sex cord-stromal tumors, sex cord-gonadal stromal tumor, ovarian stromal tumor, granulosa cell tumor, granulosa-theca tumor, Sertoli-Leydig tumor, ovarian sarcoma, ovarian carcinosarcoma, ovarian adenosarcoma, ovarian leiomyosarcoma, ovarian fibrosarcoma, Krukenberg tumor, or ovarian cyst.

In some embodiments, the pancreatic cancer is a pancreatic exocrine gland cancer, pancreatic endocrine gland cancer, or pancreatic adenocarcinoma, islet cell tumor, or neuroendocrine tumor.

In some embodiments, the prostate cancer is a prostate adenocarcinoma, prostate sarcoma, transitional cell carcinoma, small cell carcinoma, or neuroendocrine tumor.

In some embodiments, the sinus cancer is a squamous cell carcinoma, mucosa cell carcinoma, adenoid cystic cell carcinoma, acinic cell carcinoma, sinonasal undifferentiated carcinoma, nasal cavity cancer, paranasal sinus cancer, maxillary sinus cancer, ethmoid sinus cancer, or nasopharynx cancer.

In some embodiments, the skin cancer is a basal cell carcinoma, squamous cell carcinoma, melanoma, Merkel cell carcinoma, Kaposi sarcoma (KS), actinic keratosis, skin lymphoma, or keratoacanthoma.

In some embodiments, the soft tissue cancer is an angiosarcoma, dermatofibrosarcoma, epithelioid sarcoma, Ewing's sarcoma, fibrosarcoma, gastrointestinal stromal tumors (GISTs), Kaposi sarcoma, leiomyosarcoma, liposarcoma, dedifferentiated liposarcoma (DL), myxoid/round cell liposarcoma (MRCL), well-differentiated liposarcoma (WDL), malignant fibrous histiocytoma, neurofibrosarcoma, rhabdomyosarcoma (RMS), or synovial sarcoma.

In some embodiments, the spinal cancer is a spinal metastatic tumor.

In some embodiments, the stomach cancer is a stomach adenocarcinoma, stomach lymphoma, gastrointestinal stromal tumors, carcinoid tumor, gastric carcinoid tumors, Type I ECL-cell carcinoid, Type II ECL-cell carcinoid, or Type III ECL-cell carcinoid.

In some embodiments, the testicular cancer is a seminoma, non-seminoma, embryonal carcinoma, yolk sac carcinoma, choriocarcinoma, teratoma, gonadal stromal tumor, leydig cell tumor, or sertoli cell tumor.

In some embodiments, the throat cancer is a squamous cell carcinoma, adenocarcinoma, sarcoma, laryngeal cancer, pharyngeal cancer, nasopharynx cancer, oropharynx cancer, hypopharynx cancer, laryngeal cancer, laryngeal squamous cell carcinoma, laryngeal adenocarcinoma, lymphoepithelioma, spindle cell carcinoma, verrucous cancer, undifferentiated carcinoma, or lymph node cancer.

In some embodiments, the thyroid cancer is a papillary carcinoma, follicular carcinoma, Hürthle cell carcinoma, medullary thyroid carcinoma, or anaplastic carcinoma.

In some embodiments, the uterine cancer is an endometrial cancer, endometrial adenocarcinoma, endometroid carcinoma, serous adenocarcinoma, adenosquamous carcinoma, uterine carcinosarcoma, uterine sarcoma, uterine leiomyosarcoma, endometrial stromal sarcoma, or undifferentiated sarcoma.

In some embodiments, the vaginal cancer is a squamous cell carcinoma, adenocarcinoma, melanoma, or sarcoma.

In some embodiments, the vulvar cancer is a squamous cell carcinoma or adenocarcinoma.

In some embodiments, the cancer antigen is angiopoietin, BCMA, CD19, CD20, CD22, CD25 (IL2-R), CD30, CD33, CD37, CD38, CD52, CD56, CD123 (IL-3R), cMET, DLL/Notch, EGFR, EpCAM, FGF, FGF-R, GD2, HER2, Mesothelin, Nectin-4, PDGFRα, RANKL, SLAMF7, TROP2, VEGF, or VEGF-R. In some embodiments, the cancer antigen is CEA, immature laminin receptor, TAG-72, HPV E6, HPV E7, BING-4, calcium-activated chloride channel 2, cyclin-B1, 9D7, EpCAM, EphA3, Her2/neu, telomerase, mesothelin, SAP-1, surviving, a BAGE family antigen, CAGE family antigen, GAGE family antigen, MAGE family antigen, SAGE family antigen, XAGE family antigen, NY-ESO-1/LAGE-1, PRAME, SSX-2, Melan-A, MART-1, Gp100, pmel17, tyrosinase, TRP-1, TRP-2, P. polypeptide, MC1R, prostate-specific antigen, β-catenin, BRCA1, BRCA2, CDK4, CML66, fibronectin, MART-2, p53, Ras, TGF-βRII, or MUC1.

In another general aspect, provided herein is a method of targeting CD33 on the surface of a cancer cell, the method comprising exposing the cancer cell to an anti-TRDV2/anti-CD33 bispecific antibody or antigen binding fragment thereof or a pharmaceutical composition provided herein.

In certain embodiments, the C2 domain of CD33 is targeted. In other embodiments, the V domain of CD33 is targeted.

The functional activity of bispecific antibodies and antigen-binding fragments thereof that bind TRDV2 and/or CD33 can be characterized by methods known in the art and as described herein. Methods for characterizing antibodies and antigen-binding fragments thereof that bind TRDV2 and/or CD33 include, but are not limited to, affinity and specificity assays including Biacore, ELISA, and OCTETRED analysis; binding assays to detect the binding of antibodies to CD33 on cancer cells by FACS; binding assays to detect the binding of antibodies to TRDV2 on γδ T cells. According to particular embodiments, the methods for characterizing antibodies and antigen-binding fragments thereof that bind TRDV2 and/or CD33 include those described below.

In another general aspect, provided herein is a method of directing Vδ2-expressing yd T cells to a cancer cell. The methods comprise contacting the Vδ2-expressing γδ T cell with an anti-TRDV2/anti-CD33 bispecific antibody or antigen binding fragment thereof, wherein the anti-TRDV2/anti-CD33 bispecific antibody or antigen binding fragment thereof directs the Vδ2-expressing γδ T cell to a cancer cell having CD33 on its surface.

In another general aspect, provided herein is a method for inhibiting growth or proliferation of cancer cells. The methods comprise contacting the Vδ2-expressing γδ T cells with an anti-TRDV2/anti-CD33 bispecific antibody or antigen binding fragment thereof, wherein contacting the cancer cells with the anti-TRDV2/anti-CD33 bispecific antibody or antigen binding fragment thereof composition inhibits the growth or proliferation of the cancer cells.

In another general aspect, provided herein is a method of treating a cancer in a subject in need thereof, comprising administering to the subject an isolated bispecific antibody or antigen binding fragment thereof that specifically binds TRDV2 and a tumor-associated antigen presented on the surface of a tumor cell (e.g., CD33) or a pharmaceutical composition disclosed herein. The cancer can, for example, be a CD33-expressing cancer. The cancer can, for example, be a CD33-expressing cancer. The cancer can, for example, be a hematologic cancer. The hematologic cancer can, for example, be a leukemia, a lymphoma, and a myeloma. The leukemia can be an acute myeloid leukemia (AML) or an acute lymphocytic leukemia (ALL).

According to particular embodiments, provided are compositions used in the treatment of a cancer. For cancer therapy, the compositions can be used in combination with another treatment including, but not limited to, a chemotherapy, an anti-CD20 mAb, an anti-TIM-3 mAb, an anti-CTLA-4 antibody, an anti-PD-L1 antibody, an anti-PD-1 antibody, a PD-1/PD-L1 therapy, IDO, an anti-OX40 antibody, an anti-GITR antibody, an anti-CD40 antibody, an anti-CD38 antibody, cytokines, oncolytic viruses, TLR agonists, STING agonist, other immuno-oncology drugs, an antiangiogenic agent, a radiation therapy, an antibody-drug conjugate (ADC), a targeted therapy, or other anticancer drugs.

Also provided is a method of activating a γδ T cell expressing TRDV2, comprising contacting the γδ T cell with an anti-TRDV2 bispecific antibody provided herein. In some embodiments, the contacting results in an increase in CD69, CD25, and/or Granzyme B expression, as compared to a control γδ T cell expressing TRDV2.

In various embodiments of the methods provided herein, the bispecific antibody specifically binds TRDV2 and the C2 domain of CD33. In other various embodiments of the methods provided herein, the bispecific antibody specifically binds TRDV2 and the V domain of CD33.

According to certain embodiments, the pharmaceutical composition comprises an effective amount of an anti-TRDV2 bispecific antibody or antigen-binding fragment thereof provided herein.

As used herein, the term “effective amount” refers to an amount of an active ingredient or component that elicits the desired biological or medicinal response in a subject.

According to particular embodiments, an effective amount refers to the amount of therapy which is sufficient to achieve one, two, three, four, or more of the following effects: (i) reduce or ameliorate the severity of the disease, disorder or condition to be treated or a symptom associated therewith; (ii) reduce the duration of the disease, disorder or condition to be treated, or a symptom associated therewith; (iii) prevent the progression of the disease, disorder or condition to be treated, or a symptom associated therewith; (iv) cause regression of the disease, disorder or condition to be treated, or a symptom associated therewith; (v) prevent the development or onset of the disease, disorder or condition to be treated, or a symptom associated therewith; (vi) prevent the recurrence of the disease, disorder or condition to be treated, or a symptom associated therewith; (vii) reduce hospitalization of a subject having the disease, disorder or condition to be treated, or a symptom associated therewith; (viii) reduce hospitalization length of a subject having the disease, disorder or condition to be treated, or a symptom associated therewith; (ix) increase the survival of a subject with the disease, disorder or condition to be treated, or a symptom associated therewith; (xi) inhibit or reduce the disease, disorder or condition to be treated, or a symptom associated therewith in a subject; and/or (xii) enhance or improve the prophylactic or therapeutic effect(s) of another therapy.

The effective amount or dosage can vary according to various factors, such as the disease, disorder or condition to be treated, the means of administration, the target site, the physiological state of the subject (including, e.g., age, body weight, health), whether the subject is a human or an animal, other medications administered, and whether the treatment is prophylactic or therapeutic. Treatment dosages are optimally titrated to optimize safety and efficacy.

According to particular embodiments, the compositions described herein are formulated to be suitable for the intended route of administration to a subject. For example, the compositions described herein can be formulated to be suitable for intravenous, subcutaneous, or intramuscular administration.

As used herein, the terms “treat,” “treating,” and “treatment” are all intended to refer to an amelioration or reversal of at least one measurable physical parameter related to a cancer, which is not necessarily discernible in the subject, but can be discernible in the subject. The terms “treat,” “treating,” and “treatment,” can also refer to causing regression, preventing the progression, or at least slowing down the progression of the disease, disorder, or condition. In a particular embodiment, “treat,” “treating,” and “treatment” refer to an alleviation, prevention of the development or onset, or reduction in the duration of one or more symptoms associated with the disease, disorder, or condition, such as a tumor or a cancer. In a particular embodiment, “treat,” “treating,” and “treatment” refer to prevention of the recurrence of the disease, disorder, or condition. In a particular embodiment, “treat,” “treating,” and “treatment” refer to an increase in the survival of a subject having the disease, disorder, or condition. In a particular embodiment, “treat,” “treating,” and “treatment” refer to elimination of the disease, disorder, or condition in the subject.

In some embodiments, an anti-TRDV2 bispecific antibody provided herein is used in combination with a supplemental therapy.

According to particular embodiments, provided are compositions used in the treatment of a cancer. For cancer therapy, the compositions can be used in combination with another treatment including, but not limited to, a chemotherapy, an anti-CD20 mAb, an anti-TIM-3 mAb, an anti-CTLA-4 antibody, an anti-PD-L1 antibody, an anti-PD-1 antibody, a PD-1/PD-L1 therapy, IDO, an anti-OX40 antibody, an anti-GITR antibody, an anti-CD40 antibody, an anti-CD38 antibody, cytokines, oncolytic viruses, TLR agonists, STING agonist, other immuno-oncology drugs, an antiangiogenic agent, a radiation therapy, an antibody-drug conjugate (ADC), a targeted therapy, or other anticancer drugs.

As used herein, the term “in combination,” in the context of the administration of two or more therapies to a subject, refers to the use of more than one therapy. The use of the term “in combination” does not restrict the order in which therapies are administered to a subject. For example, a first therapy (e.g., a composition described herein) can be administered prior to (e.g., 5 minutes, 15 minutes, 30 minutes, 45 minutes, 1 hour, 2 hours, 4 hours, 6 hours, 12 hours, 16 hours, 24 hours, 48 hours, 72 hours, 96 hours, 1 week, 2 weeks, 3 weeks, 4 weeks, 5 weeks, 6 weeks, 8 weeks, or 12 weeks before), concomitantly with, or subsequent to (e.g., 5 minutes, 15 minutes, 30 minutes, 45 minutes, 1 hour, 2 hours, 4 hours, 6 hours, 12 hours, 16 hours, 24 hours, 48 hours, 72 hours, 96 hours, 1 week, 2 weeks, 3 weeks, 4 weeks, 5 weeks, 6 weeks, 8 weeks, or 12 weeks after) the administration of a second therapy to a subject.

TRDV2 antibodies provided herein may also be used as agents to detect cells expressing TRDV2. Thus, in another aspect, provided is a method of detecting a cell expressing TRDV2, comprising contacting a cell with a TRDV2 antibody provided herein. In certain embodiments, the cell is in a population of cells. In certain embodiments, the detecting is by ELISA. In some embodiments, the detecting is by FACS analysis. Also provided are kits comprising a TRDV2 antibody provided herein, and instructions for use.

### Enrichment and Detection Methods

In one aspect, the TRDV2 antibodies provided herein are used as agents to detect TRDV2-expressing cells. Thus, in other methods, provided is a method of detecting a cell expressing TRDV2, comprising contacting a cell with a TRDV2 antibody provided herein. In certain embodiments, the detecting is by ELISA. In some embodiments, the detecting is by FACS analysis. Also provided are kits comprising a TRDV2 antibody provided herein, and instructions for use.

Enrichment, isolation, separation, purification, sorting, selecting, capturing or detecting, or any combination thereof can be done using known technologies such as bead, microfluidics, solid support, columns, and the like. For example, TRDV2 cells may be separated or visualized using known methods when bound to the TRDV2 antibodies provided herein.

The TRDV2 antibodies or multispecific TRDV2 antibodies provided herein can be used to selectively enrich, isolate, separate, purify, sort, select, capture or detect TRDV2-expressing cells. The TRDV2 antibodies or multispecific TRDV2 antibodies provided herein may be utilized in a bispecific format, e.g. containing a first antigen binding domain that specifically binds TRDV2 and a second antigen binding domain that specifically binds a second target. In other embodiments, the multispecific TRDV2 antibodies provided herein may be utilized in a format that further incorporates a third antigen binding domain that specifically binds a third antigen (e.g., at a trispecific antibody). In other embodiments, the multispecific TRDV2 antibodies provided herein may be utilized in a format that further incorporates a fourth antigen binding domain that specifically binds a fourth antigen. (e.g., as a quadraspecific antibody).

In one aspect, provided herein is a method of enriching a TRDV2-expressing cell comprising: providing a sample comprising the TRDV2-expressing cell; contacting the sample with a TRDV2 antibody provided herein; and enriching the TRDV2-expressing cell bound to the TRDV2 antibody. In one aspect, provided herein is a method of isolating a TRDV2-expressing cell comprising: providing a sample comprising the TRDV2-expressing cell; contacting the sample with a TRDV2 antibody provided herein; and isolating the TRDV2-expressing cell bound to the TRDV2 antibody. In one aspect, provided herein is a method of separating a TRDV2-expressing cell comprising: providing a sample comprising the TRDV2-expressing cell; contacting the sample with a TRDV2 antibody provided herein; and separating the TRDV2-expressing cell bound to the TRDV2 antibody. In one aspect, provided herein is a method of purifying a TRDV2-expressing cell comprising: providing a sample comprising the TRDV2-expressing cell; contacting the sample with a TRDV2 antibody provided herein; and purifying the TRDV2-expressing cell bound to the TRDV2 antibody. In one aspect, provided herein is a method of sorting a TRDV2-expressing cell comprising: providing a sample comprising the TRDV2-expressing cell; contacting the sample with a TRDV2 antibody provided herein; and sorting the TRDV2-expressing cell bound to the TRDV2 antibody. In one aspect, provided herein is a method of selecting a TRDV2-expressing cell comprising: providing a sample comprising the TRDV2-expressing cell; contacting the sample with a TRDV2 antibody provided herein; and selecting the TRDV2-expressing cell bound to the TRDV2 antibody. In one aspect, provided herein is a method of capturing a TRDV2-expressing cell comprising: providing a sample comprising the TRDV2-expressing cell; contacting the sample with a TRDV2 antibody provided herein; and capturing the TRDV2-expressing cell bound to the TRDV2 antibody. In one aspect, provided herein is a method of detecting a TRDV2-expressing cell comprising: providing a sample comprising the TRDV2-expressing cell; contacting the sample with a TRDV2 antibody provided herein; and detecting the TRDV2-expressing cell bound to the TRDV2 antibody.

In one aspect, provided herein is a method of enriching a TRDV2-expressing cell comprising: contacting a TRDV2-expressing cell with a TRDV2 antibody provided herein; and enriching the TRDV2-expressing cell bound to the TRDV2 antibody. In one aspect, provided herein is a method of isolating a TRDV2-expressing cell comprising: contacting a TRDV2-expressing cell with a TRDV2 antibody provided herein; and isolating the TRDV2-expressing cell bound to the TRDV2 antibody. In one aspect, provided herein is a method of separating a TRDV2-expressing cell comprising: contacting a TRDV2-expressing cell with a TRDV2 antibody provided herein; and separating the TRDV2-expressing cell bound to the TRDV2 antibody. In one aspect, provided herein is a method of purifying a TRDV2-expressing cell comprising: contacting a TRDV2-expressing cell with a TRDV2 antibody provided herein; and purifying the TRDV2-expressing cell bound to the TRDV2 antibody. In one aspect, provided herein is a method of sorting a TRDV2-expressing cell comprising: contacting a TRDV2-expressing cell with a TRDV2 antibody provided herein; and sorting the TRDV2-expressing cell bound to the TRDV2 antibody. In one aspect, provided herein is a method of selecting a TRDV2-expressing cell comprising: contacting a TRDV2-expressing cell with a TRDV2 antibody provided herein; and selecting the TRDV2-expressing cell bound to the TRDV2 antibody. In one aspect, provided herein is a method of capturing a TRDV2-expressing cell comprising: contacting a TRDV2-expressing cell with a TRDV2 antibody provided herein; and capturing the TRDV2-expressing cell bound to the TRDV2 antibody. In one aspect, provided herein is a method of detecting a TRDV2-expressing cell comprising: contacting a TRDV2-expressing cell with a TRDV2 antibody provided herein; and detecting the TRDV2-expressing cell bound to the TRDV2 antibody.

In one aspect, provided herein is a method of enriching a TRDV2-expressing cell comprising: contacting a TRDV2-expressing cell with a TRDV2 antibody provided herein; and enriching the TRDV2-expressing cell based on binding of the TRDV2-expressing cell to the TRDV2 antibody. In one aspect, provided herein is a method of isolating a TRDV2-expressing cell comprising: contacting a TRDV2-expressing cell with a TRDV2 antibody provided herein; and isolating the TRDV2-expressing cell based on binding of the TRDV2-expressing cell to the TRDV2 antibody. In one aspect, provided herein is a method of separating a TRDV2-expressing cell comprising: contacting a TRDV2-expressing cell with a TRDV2 antibody provided herein; and separating the TRDV2-expressing cell based on binding of the TRDV2-expressing cell to the TRDV2 antibody. In one aspect, provided herein is a method of purifying a TRDV2-expressing cell comprising: contacting a TRDV2-expressing cell with a TRDV2 antibody provided herein; and purifying the TRDV2-expressing cell based on binding of the TRDV2-expressing cell to the TRDV2 antibody. In one aspect, provided herein is a method of sorting a TRDV2-expressing cell comprising: contacting a TRDV2-expressing cell with a TRDV2 antibody provided herein; and sorting the TRDV2-expressing cell based on binding of the TRDV2-expressing cell to the TRDV2 antibody. In one aspect, provided herein is a method of selecting a TRDV2-expressing cell comprising: contacting a TRDV2-expressing cell with a TRDV2 antibody provided herein; and selecting the TRDV2-expressing cell based on binding of the TRDV2-expressing cell to the TRDV2 antibody. In one aspect, provided herein is a method of capturing a TRDV2-expressing cell comprising: contacting a TRDV2-expressing cell with a TRDV2 antibody provided herein; and capturing the TRDV2-expressing cell based on binding of the TRDV2-expressing cell to the TRDV2 antibody. In one aspect, provided herein is a method of detecting a TRDV2-expressing cell comprising: contacting a TRDV2-expressing cell with a TRDV2 antibody provided herein; and detecting the TRDV2-expressing cell based on binding of the TRDV2-expressing cell to the TRDV2 antibody.

In certain embodiments of the methods, the TRDV2-expressing cell is a T cell. In some embodiments of the methods, the TRDV2-expressing cell is in a population of cells. In some embodiments of the methods, the TRDV2-expressing cell is in a population of lymphocytes. In some embodiments of the methods, the TRDV2-expressing cell is in a population of T cells. In some embodiments of the methods, the TRDV2-expressing cell is provided as a population of cells. In some embodiments of the methods, the TRDV2-expressing cell is provided as a population of lymphocytes. In some embodiments of the methods, the TRDV2-expressing cell is provided as a population of T cells. In some embodiments of the methods, the TRDV2-expressing cell is provided as a sample comprising a population of cells. In some embodiments of the methods, the TRDV2-expressing cell is provided as a sample comprising a population of lymphocytes. In some embodiments of the methods, the TRDV2-expressing cell is provided as a sample comprising a population of T cells. In some embodiments of the methods, the sample is a blood sample. In some embodiments of the methods, the sample is a tissue sample. In some embodiments of the methods, the sample is a tissue culture sample.

In some embodiments of the methods, the TRDV2 antibody is a multispecific TRDV2 antibody provided herein. In some embodiments of the methods, the TRDV2 antibody is a bispecific TRDV2 antibody provided herein. In some embodiments of the methods, the TRDV2 antibody is a trispecific TRDV2 antibody provided herein. In some embodiments of the methods, the TRDV2 antibody is a quadraspecific TRDV2 antibody provided herein. In certain embodiments, the TRDV2 antibody specifically binds to TRDV2. In one embodiment, the multispecific TRDV2 antibody comprises: (a) a first binding domain that binds TRDV2, and (b) a second binding domain that binds to a second target. In one embodiment, the multispecific TRDV2 antibody comprises: (a) a first binding domain that binds TRDV2, and (b) a second binding domain that binds to a second target, and (c) a third binding domain that binds to a third target. In one embodiment, the multispecific TRDV2 antibody comprises: (a) a first binding domain that binds TRDV2, and (b) a second binding domain that binds to a second target, (c) a third binding domain that binds to a third target, and (d) a fourth binding domain that binds to a fourth target. In one embodiment, the multispecific TRDV2 antibody comprises: (a) a first binding domain that specifically binds TRDV2, and (b) a second binding domain that specifically binds to a second target. In one embodiment, the multispecific TRDV2 antibody comprises: (a) a first binding domain that specifically binds TRDV2, and (b) a second binding domain that specifically binds to a second target, and (c) a third binding domain that specifically binds to a third target. In one embodiment, the multispecific TRDV2 antibody comprises: (a) a first binding domain that specifically binds TRDV2, and (b) a second binding domain that specifically binds to a second target, (c) a third binding domain that specifically binds to a third target, and (d) a fourth binding domain that specifically binds to a fourth target.

In some embodiments of the methods, the TRDV2 antibody is a multispecific TRDV2 antibody, wherein the second target is CD123. In some embodiments of the methods, the TRDV2 antibody is a multispecific TRDV2 antibody, wherein the second target is CD33. In some embodiments of the methods, the TRDV2 antibody is a multispecific TRDV2 antibody, wherein the second target is TRBC1. In some embodiments of the methods, the TRDV2 antibody is a multispecific TRDV2 antibody, wherein the second target is BCMA. In some embodiments of the methods, the TRDV2 antibody is a multispecific TRDV2 antibody, wherein the second target is PSMA.

In specific embodiments of the methods provided herein, the method uses multi-marker detection. In some embodiments, the multi-marker detection uses a multispecific TRDV2 antibody provided herein. In some embodiments, the multi-marker detection uses a bispecific TRDV2 antibody provided herein. In some embodiments, the multi-marker detection uses a trispecific TRDV2 antibody provided herein. In some embodiments, the multi-marker detection uses a quadraspecific TRDV2 antibody provided herein.

In certain embodiments of the methods provided herein, the methods are included as steps in a T cell manufacturing process. In certain embodiments, the cells are CAR-T cells. In certain embodiments of the methods provided herein, the methods are included as steps in a T cell modification process.

In certain embodiments of the methods provided herein, the methods are included as steps in a diagnostic method. In certain embodiments of the methods provided herein, the methods are included as steps in a method to quantify the TRDV2-expressing T cells.

In certain embodiments of the methods provided herein, the method further comprises expanding the enriched, isolated, separated, purified, sorted, selected, captured or detected TRDV2-expressing cells. In certain embodiments, the expanding is in vitro. In certain embodiments, the expanding is in vivo. In certain embodiments of the methods provided herein, the method further comprises growing the enriched, isolated, separated, purified, sorted, selected, captured or detected TRDV2-expressing cells. In certain embodiments, the growing is in vitro. In certain embodiments, the growing is in vivo. In certain embodiments of the methods provided herein, the method further comprises quantifying the enriched, isolated, separated, purified, sorted, selected, captured or detected TRDV2-expressing cells.

### EMBODIMENTS

Provided herein are the following non-limiting embodiments.

In one set of embodiments, provided are:
1. A bispecific antibody comprising:
   (a) a first binding domain that binds to a TRDV2 antigen, and
   (b) a second binding domain that binds to an antigen on the surface of a cancer cell.
2. The bispecific antibody of embodiment 1, wherein the first binding domain comprises:
   (i) a VH comprising a VH CDR1 having an amino acid sequence of SEQ ID NO: 1, a VH CDR2 having an amino acid sequence of SEQ ID NO: 2, and a VH CDR3 having an amino acid sequence of SEQ ID NO: 3; and
   (ii) a VL comprising a VL CDR1 having an amino acid sequence of SEQ ID NO: 4, a VL CDR2 having an amino acid sequence of SEQ ID NO: 5, and a VL CDR3 having an amino acid sequence of SEQ ID NO: 6.
3. The bispecific antibody of embodiment 2, wherein the first binding domain comprises a VH having an amino acid sequence of SEQ ID NO:7.
4. The bispecific antibody of embodiment 2, wherein the first binding domain comprises a VL having an amino acid sequence of SEQ ID NO:8.
5. The bispecific antibody of embodiment 2, wherein the first binding domain comprises a VH having an amino acid sequence of SEQ ID NO:7, and a VL having an amino acid sequence of SEQ ID NO:8.
6. The bispecific antibody of any one of embodiments 1 to 5, wherein the antigen on the surface of the cancer cell is a tumor-specific antigen, a tumor associated antigen, or a neoantigen.
7. The bispecific antibody of any one of embodiments 1 to 6, wherein the cancer cell is a cell of an adrenal cancer, anal cancer, appendix cancer, bile duct cancer, bladder cancer, bone cancer, brain cancer, breast cancer, cervical cancer, colorectal cancer, esophageal cancer, gallbladder cancer, gestational trophoblastic, head and neck cancer, Hodgkin lymphoma, intestinal cancer, kidney cancer, leukemia, liver cancer, lung cancer, melanoma, mesothelioma, multiple myeloma, neuroendocrine tumor, non-Hodgkin lymphoma, oral cancer, ovarian cancer, pancreatic cancer, prostate cancer, sinus cancer, skin cancer, soft tissue sarcoma spinal cancer, stomach cancer, testicular cancer, throat cancer, thyroid cancer, uterine cancer endometrial cancer, vaginal cancer, or vulvar cancer.
8. The bispecific antibody of any one of embodiments 1 to 7, wherein
   (i) the adrenal cancer is an adrenocortical carcinoma (ACC), adrenal cortex cancer, pheochromocytoma, or neuroblastoma;
   (ii) the anal cancer is a squamous cell carcinoma, cloacogenic carcinoma, adenocarcinoma, basal cell carcinoma, or melanoma;
   (iii) the appendix cancer is a neuroendocrine tumor (NET), mucinous adenocarcinoma, goblet cell carcinoid, intestinal-type adenocarcinoma, or signet-ring cell adenocarcinoma;
   (iv) the bile duct cancer is an extrahepatic bile duct cancer, adenocarcinomas, hilar bile duct cancer, perihilar bile duct cancer, distal bile duct cancer, or intrahepatic bile duct cancer;
   (v) the bladder cancer is transitional cell carcinoma (TCC), papillary carcinoma, flat carcinoma, squamous cell carcinoma, adenocarcinoma, small-cell carcinoma, or sarcoma;
   (vi) the bone cancer is a primary bone cancer, sarcoma, osteosarcoma, chondrosarcoma, sarcoma, fibrosarcoma, malignant fibrous histiocytoma, giant cell tumor of bone, chordoma, or metastatic bone cancer;
   (vii) the brain cancer is an astrocytoma, brain stem glioma, glioblastoma, meningioma, ependymoma, oligodendroglioma, mixed glioma, pituitary carcinoma, pituitary adenoma, craniopharyngioma, germ cell tumor, pineal region tumor, medulloblastoma, or primary CNS lymphoma;
   (viii) the breast cancer is a breast adenocarcinoma, invasive breast cancer, noninvasive breast cancer, breast sarcoma, metaplastic carcinoma, adenocystic carcinoma, phyllodes tumor, angiosarcoma, HER2-positive breast cancer, triple-negative breast cancer, or inflammatory breast cancer;
   (ix) the cervical cancer is a squamous cell carcinoma, or adenocarcinoma;
   (x) the colorectal cancer is a colorectal adenocarcinoma, primary colorectal lymphoma, gastrointestinal stromal tumor, leiomyosarcoma, carcinoid tumor, mucinous adenocarcinoma, signet ring cell adenocarcinoma, gastrointestinal carcinoid tumor, or melanoma;
   (xi) the esophageal cancer is an adenocarcinoma or squamous cell carcinoma;
   (xii) the gall bladder cancer is an adenocarcinoma, papillary adenocarcinoma, adenosquamous carcinoma, squamous cell carcinoma, small cell carcinoma, or sarcoma;
   (xiii) the gestational trophoblastic disease (GTD) is a hydatidiform mole, gestational trophoblastic neoplasia (GTN), choriocarcinoma, placental-site trophoblastic tumor (PSTT), or epithelioid trophoblastic tumor (ETT);
   (xiv) the head and neck cancer is a laryngeal cancer, nasopharyngeal cancer, hypopharyngeal cancer, nasal cavity cancer, paranasal sinus cancer, salivary gland cancer, oral cancer, oropharyngeal cancer, or tonsil cancer;
   (xv) the Hodgkin lymphoma is a classical Hodgkin lymphoma, nodular sclerosis, mixed cellularity, lymphocyte-rich, lymphocyte-depleted, or nodular lymphocyte-predominant Hodgkin lymphoma (NLPHL);
   (xvi) the intestinal cancer is a small intestine cancer, small bowel cancer, adenocarcinoma, sarcoma, gastrointestinal stromal tumors, carcinoid tumors, or lymphoma;
   (xvii) the kidney cancer is a renal cell carcinoma (RCC), clear cell RCC, papillary RCC, chromophobe RCC, collecting duct RCC, unclassified RCC, transitional cell carcinoma, urothelial cancer, renal pelvis carcinoma, or renal sarcoma;
   (xviii) the leukemia is an acute lymphocytic leukemia (ALL), acute myeloid leukemia (AML), chronic lymphocytic leukemia (CLL), chronic myeloid leukemia (CML), hairy cell leukemia (HCL), or a myelodysplastic syndrome (MDS);
   (xix) the liver cancer is a hepatocellular carcinoma (HCC), fibrolamellar HCC, cholangiocarcinoma, angiosarcoma, or liver metastasis;
   (xx) the lung cancer is a small cell lung cancer, small cell carcinoma, combined small cell carcinoma, non-small cell lung cancer, lung adenocarcinoma, squamous cell lung cancer, large-cell undifferentiated carcinoma, pulmonary nodule, metastatic lung cancer, adenosquamous carcinoma, large cell neuroendocrine carcinoma, salivary gland-type lung carcinoma, lung carcinoid, mesothelioma, sarcomatoid carcinoma of the lung, or malignant granular cell lung tumor;
   (xxi) the melanoma is a superficial spreading melanoma, nodular melanoma, acral-lentiginous melanoma, lentigo maligna melanoma, amelanotic melanoma, desmoplastic melanoma, ocular melanoma, or metastatic melanoma;
   (xxii) the mesothelioma is a pleural mesothelioma, peritoneal mesothelioma, pericardial mesothelioma, or testicular mesothelioma;
   (xxiii) the multiple myeloma is an active myeloma or smoldering myeloma;
   (xxiv) the neuroendocrine tumor, is a gastrointestinal neuroendocrine tumor, pancreatic neuroendocrine tumor, or lung neuroendocrine tumor;
   (xxv) the non-Hodgkin's lymphoma is an anaplastic large-cell lymphoma, lymphoblastic lymphoma, peripheral T cell lymphoma, follicular lymphoma, cutaneous T cell lymphoma, lymphoplasmacytic lymphoma, marginal zone B-cell lymphoma, MALT lymphoma, small-cell lymphocytic lymphoma, Burkitt lymphoma, chronic lymphocytic leukemia (CLL), small lymphocytic lymphoma (SLL), precursor T-lymphoblastic leukemia/lymphoma, acute lymphocytic leukemia (ALL), adult T cell lymphoma/leukemia (ATLL), hairy cell leukemia, B-cell lymphomas, diffuse large B-cell lymphoma (DLBCL), primary mediastinal B-cell lymphoma, primary central nervous system (CNS) lymphoma, mantle cell lymphoma (MCL), marginal zone lymphomas, mucosa-associated lymphoid tissue (MALT) lymphoma, nodal marginal zone B-cell lymphoma, splenic marginal zone B-cell lymphoma, lymphoplasmacytic lymphoma, B-cell non-Hodgkin lymphoma, T cell non-Hodgkin lymphoma, natural killer cell lymphoma, cutaneous T cell lymphoma, Alibert-Bazin syndrome, Sezary syndrome, primary cutaneous anaplastic large-cell lymphoma, peripheral T cell lymphoma, angioimmunoblastic T cell lymphoma (AITL), anaplastic large-cell lymphoma (ALCL), systemic ALCL, enteropathy-type T cell lymphoma (EATL), or hepatosplenic gamma/delta T cell lymphoma;
   (xxvi) the oral cancer is a squamous cell carcinoma, verrucous carcinoma, minor salivary gland carcinomas, lymphoma, benign oral cavity tumor, eosinophilic granuloma, fibroma, granular cell tumor, karatoacanthoma, leiomyoma, osteochondroma, lipoma, schwannoma, neurofibroma, papilloma, condyloma acuminatum, verruciform xanthoma, pyogenic granuloma, rhabdomyoma, odontogenic tumors, leukoplakia, erythroplakia, squamous cell lip cancer, basal cell lip cancer, mouth cancer, gum cancer, or tongue cancer;
   (xxvii) the ovarian cancer is a ovarian epithelial cancer, mucinous epithelial ovarian cancer, endometrioid epithelial ovarian cancer, clear cell epithelial ovarian cancer, undifferentiated epithelial ovarian cancer, ovarian low malignant potential tumors, primary peritoneal carcinoma, fallopian tube cancer, germ cell tumors, teratoma, dysgerminoma ovarian germ cell cancer, endodermal sinus tumor, sex cord-stromal tumors, sex cord-gonadal stromal tumor, ovarian stromal tumor, granulosa cell tumor, granulosa-theca tumor, Sertoli-Leydig tumor, ovarian sarcoma, ovarian carcinosarcoma, ovarian adenosarcoma, ovarian leiomyosarcoma, ovarian fibrosarcoma, Krukenberg tumor, or ovarian cyst;
   (xxviii) the pancreatic cancer is a pancreatic exocrine gland cancer, pancreatic endocrine gland cancer, or pancreatic adenocarcinoma, islet cell tumor, or neuroendocrine tumor;
   (xxix) the prostate cancer is a prostate adenocarcinoma, prostate sarcoma, transitional cell carcinoma, small cell carcinoma, or neuroendocrine tumor;
   (xxx) the sinus cancer is a squamous cell carcinoma, mucosa cell carcinoma, adenoid cystic cell carcinoma, acinic cell carcinoma, sinonasal undifferentiated carcinoma, nasal cavity cancer, paranasal sinus cancer, maxillary sinus cancer, ethmoid sinus cancer, or nasopharynx cancer; (xxxi) the skin cancer is a basal cell carcinoma, squamous cell carcinoma, melanoma, Merkel cell carcinoma, Kaposi sarcoma (KS), actinic keratosis, skin lymphoma, or keratoacanthoma;
   (xxxii) the soft tissue cancer is an angiosarcoma, dermatofibrosarcoma, epithelioid sarcoma, Ewing's sarcoma, fibrosarcoma, gastrointestinal stromal tumors (GISTs), Kaposi sarcoma, leiomyosarcoma, liposarcoma, dedifferentiated liposarcoma (DL), myxoid/round cell liposarcoma (MRCL), well-differentiated liposarcoma (WDL), malignant fibrous histiocytoma, neurofibrosarcoma, rhabdomyosarcoma (RMS), or synovial sarcoma;
   (xxxiii) the spinal cancer is a spinal metastatic tumor;
   (xxxiv) the stomach cancer is a stomach adenocarcinoma, stomach lymphoma, gastrointestinal stromal tumors, carcinoid tumor, gastric carcinoid tumors, Type I ECL-cell carcinoid, Type II ECL-cell carcinoid, or Type III ECL-cell carcinoid;
   (xxxv) the testicular cancer is a seminoma, non-seminoma, embryonal carcinoma, yolk sac carcinoma, choriocarcinoma, teratoma, gonadal stromal tumor, leydig cell tumor, or sertoli cell tumor;
   (xxxiv) the throat cancer is a squamous cell carcinoma, adenocarcinoma, sarcoma, laryngeal cancer, pharyngeal cancer, nasopharynx cancer, oropharynx cancer, hypopharynx cancer, laryngeal cancer, laryngeal squamous cell carcinoma, laryngeal adenocarcinoma, lymphoepithelioma, spindle cell carcinoma, verrucous cancer, undifferentiated carcinoma, or lymph node cancer;
   (xxxv) the thyroid cancer is a papillary carcinoma, follicular carcinoma, Hürthle cell carcinoma, medullary thyroid carcinoma, or anaplastic carcinoma;
   (xxxvi) the uterine cancer is an endometrial cancer, endometrial adenocarcinoma, endometroid carcinoma, serous adenocarcinoma, adenosquamous carcinoma, uterine carcinosarcoma, uterine sarcoma, uterine leiomyosarcoma, endometrial stromal sarcoma, or undifferentiated sarcoma;
   (xxxvii) the vaginal cancer is a squamous cell carcinoma, adenocarcinoma, melanoma, or sarcoma; or
   (xxxviii) the vulvar cancer is a squamous cell carcinoma or adenocarcinoma.
9. The bispecific antibody of any one of embodiments 1 to 8, wherein the cancer antigen is angiopoietin, BCMA, CD19, CD20, CD22, CD25 (IL2-R), CD30, CD33, CD37, CD38, CD52, CD56, CD123 (IL-3R), cMET, DLL/Notch, EGFR, EpCAM, FGF, FGF-R, GD2, HER2, Mesothelin, Nectin-4, PDGFRα, RANKL, SLAMF7, TROP2, VEGF, VEGF-R.
10. The bispecific antibody of any one of embodiments 1 to 8, wherein the cancer antigen is a CEA, immature laminin receptor, TAG-72, HPV E6, HPV E7, BING-4, calcium-activated chloride channel 2, cyclin-B1, 9D7, EpCAM, EphA3, Her2/neu, telomerase, mesothelin, SAP-1, surviving, a BAGE family antigen, CAGE family antigen, GAGE family antigen, MAGE family antigen, SAGE family antigen, XAGE family antigen, NY-ESO-1/LAGE-1, PRAME, SSX-2, Melan-A, MART-1, Gp100, pmel17, tyrosinase, TRP-1, TRP-2, P. polypeptide, MC1R, prostate-specific antigen, β-catenin, BRCA1, BRCA2, CDK4, CML66, fibronectin, MART-2, p53, Ras, TGF-βRII, or MUC1 antigen.
11. The bispecific antibody of any one of embodiments 1 to 10, wherein the TRDV2 is present on the surface of a γδ T cell.
12. The bispecific antibody of any one of embodiments 1 to 10, wherein the TRDV2 is present on the surface of a γδ T cell, and the antigen expressed on the surface of the cancer cell is a cancer antigen.
13. The bispecific antibody of embodiment 12, wherein the cancer cell is killed when the bispecific antibody binds to the TRDV2 on the surface of the γδ T cell and the antigen on the surface of the cancer cell.
14. The bispecific antibody of any one of embodiments 1 to 13, wherein the first binding domain is humanized, the second binding domain is humanized, or both the first binding domain and the second binding domain are humanized.
15. The bispecific antibody of any one of embodiments 1 to 14, wherein the bispecific antibody is an IgG antibody.
16. The bispecific antibody of embodiment 15, wherein the IgG antibody is an IgG1, IgG2, IgG3, IgG4 antibody.
17. The bispecific antibody of any one of embodiments 12 to 16, wherein the bispecific antibody induces γδ T cell dependent cytotoxicity of the cancer cell in vitro with an EC₅₀ of less than about 500 pM.
18. The bispecific antibody of embodiment 17, wherein the bispecific antibody induces γδ T cell dependent cytotoxicity of the cancer cell in vitro with an EC₅₀ of less than about 300 pM.
19. The bispecific antibody of embodiment 18, wherein the bispecific antibody induces γδ T cell dependent cytotoxicity of the cancer cell in vitro with an EC₅₀ of less than about 160 pM.
20. The bispecific antibody of any one of embodiments 17 to 19, wherein the EC₅₀ is assessed with a mixture of γδ T effector cells and target cells expressing the cancer antigen.
21. The bispecific antibody of embodiment 20, wherein the effector cell to target cell ratio is about 0.01 to 1 to about 5 to 1.
22. The bispecific antibody of embodiment 21, wherein the effector cell to target cell ratio is about 0.1 to 1 to about 2 to 1.
23. The bispecific antibody of embodiment 22, wherein the effector cell to target cell ratio is about 1:1.
24. The bispecific antibody of any one of embodiments 1 to 23, wherein the bispecific antibody is multivalent.
25. The bispecific antibody of embodiment 24, wherein the bispecific antibody is capable of binding at least three antigens.
26. The bispecific antibody of embodiment 25, wherein the bispecific antibody is capable of binding at least five antigens.
27. A bispecific antibody comprising: a first means capable of binding TRDV2 on the surface of the γδ T cell; and a second means capable of binding a cancer antigen.
28. The bispecific antibody of embodiment 27, wherein the cancer antigen is on the surface of a cancer cell.
29. A nucleic acid encoding the bispecific antibody of any one of embodiments 1 to 28.
30. A vector comprising the nucleic acid of embodiment 29.
31. A host cell comprising the vector of embodiment 30.
32. A kit comprising the vector of embodiment 30 and packaging for the same 33. A pharmaceutical composition comprising the bispecific antibody of any one of embodiments 1 to 28, and a pharmaceutically acceptable carrier.
34. A method of producing the pharmaceutical composition of embodiment 33, comprising combining the bispecific antibody with a pharmaceutically acceptable carrier to obtain the pharmaceutical composition.
35. A process for making an antibody that binds to more than one target molecule, the molecule comprising: a step for performing a function of obtaining a binding domain capable of binding to TRDV2 antigen on a γδ T cell; a step for performing a function of obtaining a binding domain capable of binding to an antigen on the surface of a cancer cell; and a step for performing a function of providing an antibody capable of binding to a TRDV2 antigen on a γδ T cell and an antigen on the surface of a cancer cell.
36. The process of embodiment 35, wherein the step for performing a function of obtaining a binding domain capable of binding to an antigen on the surface of a cancer cell is repeated n times and further comprising n steps for performing a function of providing a binding domain capable of binding to a TRDV2 antigen on a γδ T cell and n number of target molecules, wherein n is at least 2.
37. A method of directing a γδ T cell expressing TRDV2 to a cancer cell, the method comprising contacting the γδ T cell with the bispecific antibody of any one of embodiments 1 to 28, wherein the contacting directs the γδ T cell to the cancer cell.
38. A method of inhibiting growth or proliferation of cancer cells expressing a cancer antigen on the cell surface, the method comprising contacting the cancer cells with the bispecific antibody of any one of embodiments 1 to 28, wherein contacting the cancer cells with the pharmaceutical composition inhibits growth or proliferation of the cancer cells.
39. The method of embodiment 38, wherein the cancer cells are in the presence of a yd T cell expressing TRDV2 while in contact with the bispecific antibody.
40. A method for eliminating cancer cells or treating cancer in a subject, comprising administering an effective amount of the bispecific antibody of any one of embodiments 1 to 28 to the subject.
41. The method of embodiment 40, wherein the subject is a subject in need thereof.
42. The method of embodiment 40 or 41, wherein the subject is a human.
43. A method of activating a γδ T cell expressing TRDV2, comprising contacting the γδ T cell with the bispecific antibody of any one of embodiments 1 to 28.
44. The method of embodiment 43, wherein the contacting results in an increase in CD69, CD25, and/or Granzyme B expression, as compared to a control γδ T cell expressing TRDV2.
45. The method of embodiment 38 or 39, wherein
   (i) the cancer cells are cells of an adrenal cancer, anal cancer, appendix cancer, bile duct cancer, bladder cancer, bone cancer, brain cancer, breast cancer, cervical cancer, colorectal cancer, esophageal cancer, gallbladder cancer, gestational trophoblastic, head and neck cancer, Hodgkin lymphoma, intestinal cancer, kidney cancer, leukemia, liver cancer, lung cancer, melanoma, mesothelioma, multiple myeloma, neuroendocrine tumor, non-Hodgkin lymphoma, oral cancer, ovarian cancer, pancreatic cancer, prostate cancer, sinus cancer, skin cancer, soft tissue sarcoma spinal cancer, stomach cancer, testicular cancer, throat cancer, thyroid cancer, uterine cancer endometrial cancer, vaginal cancer, or vulvar cancer;
   (ii) the cancer antigen is angiopoietin, BCMA, CD19, CD20, CD22, CD25 (IL2-R), CD30, CD33, CD37, CD38, CD52, CD56, CD123 (IL-3R), cMET, DLL/Notch, EGFR, EpCAM, FGF, FGF-R, GD2, HER2, Mesothelin, Nectin-4, PDGFRα, RANKL, SLAMF7, TROP2, VEGF, or VEGF-R; and/or
   (iii) the cancer antigen is CEA, immature laminin receptor, TAG-72, HPV E6, HPV E7, BING-4, calcium-activated chloride channel 2, cyclin-B1, 9D7, EpCAM, EphA3, Her2/neu, telomerase, mesothelin, SAP-1, surviving, a BAGE family antigen, CAGE family antigen, GAGE family antigen, MAGE family antigen, SAGE family antigen, XAGE family antigen, NY-ESO-1/LAGE-1, PRAME, SSX-2, Melan-A, MART-1, Gp100, pmel17, tyrosinase, TRP-1, TRP-2, P. polypeptide, MC1R, prostate-specific antigen, β-catenin, or BRCA1.
46. The method of any one of embodiments 40 to 44, wherein the cancer is an adrenal cancer, anal cancer, appendix cancer, bile duct cancer, bladder cancer, bone cancer, brain cancer, breast cancer, cervical cancer, colorectal cancer, esophageal cancer, gallbladder cancer, gestational trophoblastic, head and neck cancer, Hodgkin lymphoma, intestinal cancer, kidney cancer, leukemia, liver cancer, lung cancer, melanoma, mesothelioma, multiple myeloma, neuroendocrine tumor, non-Hodgkin lymphoma, oral cancer, ovarian cancer, pancreatic cancer, prostate cancer, sinus cancer, skin cancer, soft tissue sarcoma spinal cancer, stomach cancer, testicular cancer, throat cancer, thyroid cancer, uterine cancer endometrial cancer, vaginal cancer, or vulvar cancer.
47. An isolated TRDV2 bispecific antibody or antigen binding fragment thereof, the isolated TRDV2 bispecific antibody or antigen binding fragment thereof comprising:
   a. a first heavy chain (HC1);
   b. a second heavy chain (HC2);
   c. a first light chain (LC1); and
   d. a second light chain (LC2),
   wherein HC1 is associated with LC1 and HC2 is associated with LC2, and wherein HC1 comprises a heavy chain complementarity determining region 1 (HCDR1), HCDR2, and HCDR3 comprising the amino acid sequences of SEQ ID NO: 1, SEQ ID NO:2, and SEQ ID NO:3, respectively, and LC1 comprises a light chain complementarity determining region 1 (LCDR1), LCDR2, and LCDR3 comprising the amino acid sequences of SEQ ID NO:4, SEQ ID NO: 5, and SEQ ID NO:6, respectively, to form a binding site for a first antigen, and wherein HC2 and LC2 form a binding site for a second antigen.
48. The isolated TRDV2 bispecific antibody or antigen binding fragment thereof embodiment 47, wherein HC1 comprises an amino acid sequence having at least 95% identity to an amino acid sequence of SEQ ID NO:7, and LC1 comprises an amino acid sequence having at least 95% identity to the amino acid sequence of SEQ ID NO:8.
49. The isolated TRDV2 bispecific antibody or antigen binding fragment thereof embodiment 48, wherein HC1 comprises the amino acid sequence of SEQ ID NO: 7, and LC1 comprises the amino acid sequence of SEQ ID NO:8.
50 The isolated TRDV2 bispecific antibody or antigen binding fragment thereof any one of embodiments 47 to 49, wherein the binding site for a first antigen binds to TRDV2 on a γδ T cell.
51. The isolated TRDV2 bispecific antibody or antigen binding fragment thereof any one of embodiments 47 to 50, wherein the binding site for a second antigen binds to a cancer antigen present on the surface of a cancer cell.
52. The isolated TRDV2 bispecific antibody or antigen binding fragment of embodiment 51, wherein the binding of the bispecific antibody to TRDV2 present on the surface of the γδ T cell and the binding of the cancer antigen present on the surface of the cancer cell results in the killing of the cancer cell.
53. The isolated TRDV2 bispecific antibody or antigen binding fragment of any one of embodiments 47 to 52, wherein HC1 and LC1 are humanized.
54. The isolated TRDV2 bispecific antibody or antigen binding fragment thereof any one of embodiments 47 to 53, wherein HC2 and LC2 bind to CD33.
55. The isolated TRDV2 bispecific antibody or antigen binding fragment thereof any one of embodiments 47 to 54, wherein the bispecific antibody or antigen binding fragment thereof is an IgG1, an IgG2, an IgG3, or an IgG4 isotype.
56. The isolated TRDV2 bispecific antibody or antigen binding fragment thereof any one of embodiments 47 to 55, wherein the bispecific antibody or antigen binding fragment thereof is an IgG4 isotype.
57. The isolated TRDV2 bispecific antibody or antigen binding fragment thereof any one of embodiments 47 to 56, wherein the bispecific antibody or antigen binding fragment thereof induces γδ T cell dependent cytotoxicity of a cancer cell in vitro with an EC₅₀ of less than about 500 pM.
58. The isolated TRDV2 bispecific antibody or antigen binding fragment thereof embodiment 57, wherein the bispecific antibody or antigen binding fragment thereof induces γδ T cell dependent cytotoxicity of a cancer cell in vitro with an EC₅₀ of less than about 300 pM.
59. The isolated TRDV2 bispecific antibody or antigen binding fragment thereof embodiment 57, wherein the bispecific antibody or antigen binding fragment thereof induces γδ T cell dependent cytotoxicity of a cancer cell in vitro with an EC₅₀ of less than about 160 pM.
60. The isolated TRDV2 bispecific antibody or antigen binding fragment thereof any one of embodiments 57 to 59, wherein the EC₅₀ is assessed with a mixture of γδ T effector cells and Kasumi3 AML target cells.
61. The isolated TRDV2 bispecific antibody or antigen binding fragment thereof embodiment 60, wherein the effector cell to target cell ratio is about 0.01 to 1 to about 5 to 1.
62. The isolated TRDV2 bispecific antibody or antigen binding fragment thereof embodiment 61, wherein the effector cell to target cell ratio is about 0.1 to 1 to about 2 to 1.
63. The isolated TRDV2 bispecific antibody or antigen binding fragment thereof embodiment 62, wherein the effector cell to target cell ratio is about 1:1.
64. The isolated TRDV2 bispecific antibody or antigen binding fragment thereof any one of embodiments 47 to 63, wherein the bispecific antibody or antigen binding fragment thereof is multivalent.
65 The isolated TRDV2 bispecific antibody or antigen binding fragment thereof embodiment 64, wherein the bispecific antibody or antigen binding fragment thereof is capable of binding at least three antigens.
66 The isolated TRDV2 bispecific antibody or antigen binding fragment thereof embodiment 64, wherein the bispecific antibody or antigen binding fragment thereof is capable of binding at least five antigens.
67 An isolated γδ T cell bispecific antibody or antigen binding fragment thereof, the isolated γδ T cell bispecific antibody or antigen binding fragment thereof comprising:
   a. a HC1;
   b. a HC2;
   c. a LC1; and
   d. a LC2,
   wherein HC1 is associated with LC1 and HC2 is associated with LC2, wherein HC1 and LC1 form a binding site for a first antigen on a γδ T cell, and wherein HC2 and LC2 form a binding site for a second antigen.
68. A bispecific antibody comprising: a first means capable of specifically binding a T cell receptor gamma chain; and a second means capable of specifically binding a target molecule that is not a T cell receptor gamma chain.
69. A process for making a molecule capable of specifically binding to more than one target molecule, the molecule comprising: a step for performing a function of obtaining an oligopeptide or polypeptide capable of binding to a T cell receptor gamma chain; a step for performing a function of obtaining an oligopeptide or polypeptide capable of binding to a target; and a step for performing a function of providing a molecule capable of specifically binding to a T cell receptor gamma chain and a target molecule.
70. The process of embodiment 69, wherein the step for performing a function of obtaining an oligopeptide or polypeptide capable of binding to a target is repeated n times and further comprising n steps for performing a function of providing a molecule capable of specifically binding to a T cell receptor gamma chain and n number of target molecules, wherein n is at least 2.
71. An isolated anti-TRDV2/anti-CD33 bispecific antibody or antigen binding fragment thereof comprising:
   a. a HC1;
   b. a HC2
   c. a LC1; and
   d. a LC2,
   wherein HC1 is associated with LC1 and HC2 is associated with LC2, and wherein HC1 comprises a HCDR1, HCDR2, and HCDR3 comprising the amino acid sequences of SEQ ID NO:1, SEQ ID NO:2, and SEQ ID NO:3, respectively, and LC1 comprises a LCDR1, LCDR2, and LCDR3 comprising the amino acid sequences of SEQ ID NO:4, SEQ ID NO:5, and SEQ ID NO:6, respectively, to form a binding site for a first antigen that specifically binds Vδ2, and
   wherein HC2 comprises a HCDR1, HCDR2, and HCDR3 comprising the amino acid sequences of SEQ ID NO:9, SEQ ID NO: 10, and SEQ ID NO:11, respectively, and LC2 comprises a LCDR1, LCDR2, and LCDR3 comprising the amino acid sequences of SEQ ID NO:12, SEQ ID NO:13, and SEQ ID NO: 14, respectively, to form a binding site for a second antigen that specifically binds CD33.
72. The isolated anti-TRDV2/anti-CD33 bispecific antibody or antigen binding fragment thereof embodiment 71, wherein HC1 comprises an amino acid sequence having at least 95% identity to an amino acid sequence of SEQ ID NO: 7, and LC1 comprises an amino acid sequence having at least 95% identity to the amino acid sequence of SEQ ID NO:8.
73 The isolated anti-TRDV2/anti-CD33 bispecific antibody or antigen binding fragment thereof embodiment 72, wherein HC1 comprises the amino acid sequence of SEQ ID NO:7, and LC1 comprises the amino acid sequence of SEQ ID NO:8.
74. The isolated anti-TRDV2/anti-CD33 bispecific antibody or antigen binding fragment thereof any one of embodiments 71 to 73, wherein HC2 comprises an amino acid sequence having at least 95% identity to the amino acid sequence of SEQ ID NO: 15 and LC2 comprises an amino acid sequence having at least 95% identity to the amino acid sequence of SEQ ID NO:16.
75 The isolated anti-TRDV2/anti-CD33 bispecific antibody or antigen binding fragment thereof embodiment 74, wherein HC2 comprises the amino acid sequence of SEQ ID NO:15 and LC2 comprises the amino acid sequence of SEQ ID NO:16.
76. The isolated anti-TRDV2/anti-CD33 bispecific antibody or antigen binding fragment thereof any one of embodiments 71 to 75, wherein the TRDV2 is on the surface of a γδ T cell.
77. The isolated anti-TRDV2/anti-CD33 bispecific antibody or antigen binding fragment thereof any one of embodiments 73 to 76, wherein the CD33 is on the surface of a tumor cell or a CD34+ stem cell.
78. The isolated anti-TRDV2/anti-CD33 bispecific antibody or antigen binding fragment thereof any one of embodiments 71 to 77, wherein the binding of the bispecific antibody to TRDV2 present on the surface of the γδ T cell and the binding of the CD33 on the surface of the cancer cell results in the killing of the cancer cell.
79 The isolated anti-TRDV2/anti-CD33 bispecific antibody or antigen binding fragment thereof any one of embodiments 71 to 78, wherein HC1 and LC1 are humanized.
80. The isolated anti-TRDV2/anti-CD33 bispecific antibody or antigen binding fragment thereof any one of embodiments 71 to 79, wherein HC2 and LC2 are humanized.
81. The isolated anti-TRDV2/anti-CD33 bispecific antibody or antigen binding fragment thereof any one of embodiments 71 to 80, wherein the bispecific antibody or antigen binding fragment thereof is an IgG1, an IgG2, an IgG3, or an IgG4 isotype.
82. The isolated anti-TRDV2/anti-CD33 bispecific antibody or antigen binding fragment thereof any one of embodiments 71 to 81, wherein the bispecific antibody or antigen binding fragment thereof is an IgG4 isotype.
83. The isolated anti-TRDV2/anti-CD33 bispecific antibody or antigen binding fragment thereof any one of embodiments 71 to 82, wherein the bispecific antibody or antigen binding fragment thereof induces γδ T cell dependent cytotoxicity of a cancer cell in vitro with an EC₅₀ of less than about 500 pM.
84. The isolated anti-TRDV2/anti-CD33 bispecific antibody or antigen binding fragment thereof embodiment 83, wherein the bispecific antibody or antigen binding fragment thereof induces γδ T cell dependent cytotoxicity of a cancer cell in vitro with an EC₅₀ of less than about 300 pM.
85. The isolated anti-TRDV2/anti-CD33 bispecific antibody or antigen binding fragment thereof embodiment 83, wherein the bispecific antibody or antigen binding fragment thereof induces γδ T cell dependent cytotoxicity of a cancer cell in vitro with an EC₅₀ of less than about 160 pM.
86 The isolated anti-TRDV2/anti-CD33 bispecific antibody or antigen binding fragment thereof any one of embodiments 83 to 85, wherein the EC₅₀ is assessed with a mixture of γδ T effector cells and Kasumi3 AML target cells.
87. The isolated anti-TRDV2/anti-CD33 bispecific antibody or antigen binding fragment thereof embodiment 86, wherein the effector cell to target cell ratio is about 0.01 to 1 to about 5 to 1.
88. The isolated anti-TRDV2/anti-CD33 bispecific antibody or antigen binding fragment thereof embodiment 87, wherein the effector cell to target cell ratio is about 0.1 to 1 to about 2 to 1.
89. The isolated anti-TRDV2/anti-CD33 bispecific antibody or antigen binding fragment thereof embodiment 88, wherein the effector cell to target cell ratio is about 1:1.
90. A method of making the isolated anti-TRDV2/anti-CD33 bispecific antibody or antigen binding fragment thereof any one of embodiments 71 to 89, the method comprising culturing a cell comprising a nucleic acid encoding the anti-TRDV2/anti-CD33 bispecific antibody or antigen binding fragment thereof under conditions to produce the bispecific antibody or antigen binding fragment thereof and recovering the bispecific antibody or antigen binding fragment thereof.
91. An isolated TRDV2 bispecific antibody or antigen epitope binding fragment thereof, wherein the isolated TRDV2 bispecific antibody or antigen epitope binding fragment thereof comprises a binding site for a first antigen and a binding site for a second antigen, wherein the binding site for the first antigen binds a TRDV2 epitope on a γδ T cell and the binding site for the second antigen binds an epitope of the second antigen on a surface of a target cell, and the binding of the TRDV2 epitope on the γδ T cell and the binding of the second antigen epitope on the target cell results in the killing of the target cell.
92. An isolated TRDV2 bispecific antibody or antigen binding fragment thereof, wherein the isolated TRDV2 bispecific antibody or antigen binding fragment thereof comprises:
   a. a HC1;
   b. a HC2;
   c. a LC1; and
   d. a LC2,
   wherein HC1 is associated with LC1 and HC2 is associated with LC2, and wherein HC1 comprises a HCDR1, HCDR2, and HCDR3 comprising the amino acid sequences of SEQ ID NO:1, SEQ ID NO:2, and SEQ ID NO:3, respectively, and LC1 comprises a LCDR1, LCDR2, and LCDR3 comprising the amino acid sequences of SEQ ID NO:4, SEQ ID NO:5, and SEQ ID NO:6, respectively, to form the binding site for the first antigen, and wherein HC2 and LC2 form the binding site for the second antigen epitope.
93. The isolated TRDV2 bispecific antibody or antigen binding fragment thereof embodiment 92, wherein HC1 comprises an amino acid sequence having at least 95% identity to an amino acid sequence of SEQ ID NO:7, and LC1 comprises an amino acid sequence having at least 95% identity to the amino acid sequence of SEQ ID NO:8.
94. The isolated TRDV2 bispecific antibody or antigen binding fragment thereof embodiment 93, wherein HC1 comprises the amino acid sequence of SEQ ID NO: 7, and LC1 comprises the amino acid sequence of SEQ ID NO:8.
95. The isolated TRDV2 bispecific antibody or antigen binding fragment of any one of embodiments 92 to 94, wherein HC1 and LC1 are humanized.
96. The isolated TRDV2 bispecific antibody or antigen binding fragment thereof any one of embodiments 92 to 95, wherein HC2 and LC2 bind to a CD33 epitope.
97. The isolated TRDV2 bispecific antibody or antigen binding fragment thereof embodiment 96, wherein HC2 comprises an amino acid sequence having at least 95% identity to the amino acid sequence of SEQ ID NO: 15 and LC2 comprises an amino acid sequence having at least 95% identity to the amino acid sequence of SEQ ID NO:16.
98. The isolated TRDV2 bispecific antibody or antigen binding fragment thereof embodiment 97, wherein HC2 comprises the amino acid sequence of SEQ ID NO: 15 and LC2 comprises the amino acid sequence of SEQ ID NO:16.
99. The isolated TRDV2 bispecific antibody or antigen binding fragment thereof any one of embodiments 91 to 98, wherein the bispecific antibody or antigen binding fragment thereof is an IgG1, an IgG2, an IgG3, or an IgG4 isotype.
100. The isolated TRDV2 bispecific antibody or antigen binding fragment thereof any one of embodiments 91 to 99, wherein the bispecific antibody or antigen binding fragment thereof is an IgG4 isotype.
101. The isolated TRDV2 bispecific antibody or antigen binding fragment thereof any one of embodiments 91 to 100, wherein the bispecific antibody or antigen binding fragment thereof induces γδ T cell dependent cytotoxicity of a cancer cell in vitro with an EC₅₀ of less than about 500 pM.
102. The isolated TRDV2 bispecific antibody or antigen binding fragment thereof embodiment 101, wherein the bispecific antibody or antigen binding fragment thereof induces γδ T cell dependent cytotoxicity of a cancer cell in vitro with an EC₅₀ of less than about 300 pM.
103. The isolated TRDV2 bispecific antibody or antigen binding fragment thereof embodiment 102, wherein the bispecific antibody or antigen binding fragment thereof induces γδ T cell dependent cytotoxicity of a cancer cell in vitro with an EC₅₀ of less than about 160 pM.
104. The isolated TRDV2 bispecific antibody or antigen binding fragment thereof any one of embodiments 101 to 103, wherein the EC₅₀ is assessed with a mixture of γδ T effector cells and Kasumi3 AML target cells.
105. The isolated TRDV2 bispecific antibody or antigen binding fragment thereof embodiment 104 wherein the effector cell to target cell ratio is about 0.01 to 1 to about 5 to 1.
106. The isolated TRDV2 bispecific antibody or antigen binding fragment thereof embodiment 105, wherein the effector cell to target cell ratio is about 0.1 to 1 to about 2 to 1.
107. The isolated TRDV2 bispecific antibody or antigen binding fragment thereof embodiment 106, wherein the effector cell to target cell ratio is about 1:1.
108. An isolated γδ T cell bispecific antibody or antigen binding fragment thereof, wherein the isolated γδ T cell bispecific antibody or antigen binding fragment thereof comprises a binding site for a first antigen epitope and a binding site for a second antigen epitope, wherein the binding site for the first antigen epitope binds a first antigen on a γδ T cell and the binding site for the second antigen epitope binds the second antigen epitope on a surface of a target cell, and the binding of the first antigen epitope on the γδ T cell and the binding of the second antigen epitope on the target cell results in the killing of the target cell.
109. An isolated nucleic acid encoding a TRDV2 bispecific antibody or antigen binding fragment thereof, the isolated TRDV2 bispecific antibody or antigen binding fragment thereof comprising:
   a. a HC1;
   b. a HC2;
   c. a LC1; and
   d. a LC2,
   wherein HC1 is associated with LC1 and HC2 is associated with LC2, and wherein HC1 comprises a HCDR1, HCDR2, and HCDR3 comprising the amino acid sequences of SEQ ID NO:1, SEQ ID NO:2, and SEQ ID NO:3, respectively, and LC1 comprises a LCDR1, LCDR2, and LCDR3 comprising the amino acid sequences of SEQ ID NO:4, SEQ ID NO:5, and SEQ ID NO:6, respectively, to form a binding site for a first antigen, and wherein HC2 and LC2 form a binding site for a second antigen.
110. The isolated nucleic acid of embodiment 109, wherein HC1 comprises an amino acid sequence having at least 95% identity to the amino acid sequence of SEQ ID NO:7, and LC1 comprises an amino acid sequence having at least 95% identity to the amino acid sequence of SEQ ID NO:8.
111. The isolated nucleic acid of embodiment 110, wherein HC1 comprises the amino acid sequence of SEQ ID NO:7, and LC1 comprises the amino acid sequence of SEQ ID NO:8.
112. The isolated nucleic acid of any one of embodiments 109 to 111, wherein the binding site for a first antigen binds to TRDV2 on a γδ T cell.
113. The isolated nucleic acid of any one of embodiments 109 to 112, wherein the binding site for a second antigen binds to a cancer antigen present on the surface of a cancer cell.
114. The isolated nucleic acid of embodiment 113, wherein the binding of the bispecific antibody to TRDV2 present on the surface of the γδ T cell and the binding of the cancer antigen present on the surface of the cancer cell results in the killing of the cancer cell.
115. The isolated nucleic acid of any one of embodiments 109 to 114, wherein HC1 and LC1 are humanized.
116. The isolated nucleic acid of any one of embodiments 109 to 115, wherein HC2 and LC2 bind to CD33.
117. The isolated nucleic acid of any one of embodiments 109 to 116, wherein the bispecific antibody or antigen binding fragment thereof is an IgG1, an IgG2, an IgG3, or an IgG4 isotype.
118. The isolated nucleic acid of any one of embodiments 109 to 117, wherein the bispecific antibody or antigen binding fragment thereof is an IgG4 isotype.
119. The isolated nucleic acid of any one of embodiments 109 to 118, wherein the bispecific antibody or antigen binding fragment thereof induces γδ T cell dependent cytotoxicity of a cancer cell in vitro with an EC₅₀ of less than about 500 pM.
120. The isolated nucleic acid of embodiment 119, wherein the bispecific antibody or antigen binding fragment thereof induces γδ T cell dependent cytotoxicity of a cancer cell in vitro with an EC₅₀ of less than about 300 pM.
121. The isolated nucleic acid of embodiment 119, wherein the bispecific antibody or antigen binding fragment thereof induces γδ T cell dependent cytotoxicity of a cancer cell in vitro with an EC₅₀ of less than about 160 pM.
122. The isolated nucleic acid of any one of embodiments 119 to 121, wherein the EC₅₀ is assessed with a mixture of γδ T effector cells and Kasumi3 AML target cells.
123. The isolated nucleic acid of embodiment 122, wherein the effector cell to target cell ratio is about 0.01 to 1 to about 5 to 1.
124. The isolated nucleic acid of embodiment 123, wherein the effector cell to target cell ratio is about 0.1 to 1 to about 2 to 1.
125. The isolated nucleic acid of embodiment 124, wherein the effector cell to target cell ratio is about 1:1.
126. The isolated nucleic acid of any one of embodiments 109 to 125, wherein the bispecific antibody or antigen binding fragment thereof is multivalent.
127. The isolated nucleic acid of embodiment 126, wherein the bispecific antibody or antigen binding fragment thereof is capable of binding at least three antigens.
128. The isolated nucleic acid of embodiment 126, wherein the bispecific antibody or antigen binding fragment thereof is capable of binding at least five antigens.
129. A vector comprising the isolated nucleic acid of any one of embodiments 109 to 128.
130. A host cell comprising the vector of embodiment 129.
131. A kit comprising the vector of embodiment 129 and packaging for the same.
132. A pharmaceutical composition comprising an isolated TRDV2 bispecific antibody or antigen binding fragment thereof, the isolated TRDV2 bispecific antibody or antigen binding fragment thereof comprising:
   a. a HC1;
   b. a HC2;
   c. a LC1; and
   d. a LC2,
   wherein HC1 is associated with LC1 and HC2 is associated with LC2, and wherein HC1 comprises a HCDR1, HCDR2, and HCDR3 comprising the amino acid sequences of SEQ ID NO:1, SEQ ID NO:2, and SEQ ID NO:3, respectively, and LC1 comprises a LCDR1, LCDR2, and LCDR3 comprising the amino acid sequences of SEQ ID NO:4, SEQ ID NO:5, and SEQ ID NO:6, respectively, to form a binding site for a first antigen, and wherein HC2 and LC2 form a binding site for a second antigen, and a pharmaceutically acceptable carrier.
133. The pharmaceutical composition of embodiment 132, wherein HC1 comprises an amino acid sequence having at least 95% identity to the amino acid sequence of SEQ ID NO:7, and LC1 comprises an amino acid sequence having at least 95% identity to the amino acid sequence of SEQ ID NO:8.
134. The pharmaceutical composition of embodiment 133, wherein HC1 comprises the amino acid sequence of SEQ ID NO:7, and LC1 comprises the amino acid sequence of SEQ ID NO:8.
135. The pharmaceutical composition of any one of embodiments 132 to 134, wherein the binding site for a first antigen binds to TRDV2 on a γδ T cell.
136. The pharmaceutical composition of any one of embodiments 132 to 135, wherein the binding site for a second antigen binds to a cancer antigen present on the surface of a cancer cell.
137. The pharmaceutical composition of embodiment 136, wherein the binding of the bispecific antibody to TRDV2 present on the surface of the γδ T cell and the binding of the cancer antigen present on the surface of the cancer cell results in the killing of the cancer cell.
138. The pharmaceutical composition of any one of embodiments 132 to 137, wherein HC1 and LC1 are humanized.
139. The pharmaceutical composition of any one of embodiments 132 to 138, wherein HC2 and LC2 bind to CD33.
140. The pharmaceutical composition of any one of embodiments 132 to 139, wherein the bispecific antibody or antigen binding fragment thereof is an IgG1, an IgG2, an IgG3, or an IgG4 isotype.
141. A method of directing a Vδ2-expressing γδ T cell to a cancer cell, the method comprising contacting a Vδ2-expressing γδ T cell with the pharmaceutical composition of any one of embodiments 132 to 140, wherein contacting the Vδ2-expressing γδ T cell with the pharmaceutical composition directs the Vδ2-expressing γδ T cell to a cancer cell.
142. A method of inhibiting growth or proliferation of cancer cells expressing a cancer antigen on the cell surface, the method comprising contacting the cancer cells with the pharmaceutical composition of any one of embodiments 132 to 140, wherein contacting the cancer cells with the pharmaceutical composition inhibits growth or proliferation of the cancer cells.
143. The method of embodiment 142, wherein the cancer cell is in the presence of a Vδ2-expressing γδ T cell while in contact with anti-TRDV2 bispecific antibody or antigen binding fragment thereof.
144. A method for treating a cancer in a subject in need thereof, the method comprising:
   a. identifying a subject in need of cancer treatment; and
   b. administering to the subject in need thereof the pharmaceutical composition of any one of embodiments 132 to 140,
   wherein administering the pharmaceutical composition to the subject in need thereof treats the cancer in the subject.
145. A method of activating a Vδ2-expressing γδ T cell, the method comprising contacting the Vδ2-expressing γδ T cell with the pharmaceutical composition of any one of embodiments 132 to 140, wherein contacting the Vδ2-expressing γδ T cell with the pharmaceutical composition results in an increase in CD69, CD25, and/or Granzyme B expression as compared to a control Vδ2-expressing γδ T cell.
146. A method of producing the pharmaceutical composition of any one of embodiments 132 to 140, the method comprising combining the bispecific antibody or antigen binding fragment thereof with a pharmaceutically acceptable carrier to obtain the pharmaceutical composition.

Provided in the Examples herein are exemplary multi-specific (bispecific) antibodies that bind to TRDV2 and CD33. These Examples are illustrative of exemplary bispecific antibodies that can effectively target a variety of cells and tissues in a subject.

In some embodiments, provided herein is a bispecific antibody comprising: (a) a first binding domain that binds to a TRDV2 antigen, and (b) a second binding domain that binds to a second target antigen. In some embodiments, provided herein is a bispecific antibody comprising: (a) a first binding domain that specifically binds to a TRDV2 antigen, and (b) a second binding domain that specifically binds to a second target antigen. In some embodiments, provided herein is a bispecific antibody comprising: (a) a first binding domain that binds to a first epitope on a TRDV2 antigen, and (b) a second binding domain that binds to a second epitope on a second target antigen. In some embodiments, provided herein is a bispecific antibody comprising: (a) a first binding domain that specifically binds to a first epitope on a TRDV2 antigen, and (b) a second binding domain that specifically binds to a second epitope on a second target antigen. In certain embodiments, the second target antigen is CD33.

Exemplary binding agents that bind to TRDV2, as well as exemplary binding agents that bind to CD33 are provided elsewhere herein, for example in the Examples, as well as Table 1.1, Table 1.2, and Tables 1-8.

Particular embodiments of this invention are described herein. Upon reading the foregoing description, variations of the disclosed embodiments may become apparent to individuals working in the art, and it is expected that those skilled artisans may employ such variations as appropriate. Accordingly, it is intended that the invention be practiced otherwise than as specifically described herein, and that the invention includes all modifications and equivalents of the subject matter recited in the claims appended hereto as permitted by applicable law. Moreover, any combination of the above-described elements in all possible variations thereof is encompassed by the invention unless otherwise indicated herein or otherwise clearly contradicted by context. A number of embodiments of the invention have been described. Nevertheless, it will be understood that various modifications may be made without departing from the spirit and scope of the invention. Accordingly, the descriptions in the Examples section are intended to illustrate but not limit the scope of invention described in the claims.

### EXAMPLES

### Example 1: Production of Bispecific Antibodies that Bind γδ T Cells

1.1: Production of MABS that Bind γδ T Cell Antigens

Antigens or portions of antigens specific for γδ T cells are used to immunize an animal (e.g., a mouse or a rabbit). To generate the γδ T cell monoclonal antibodies, peripheral blood mononuclear cells are isolated from the whole blood of the immunized animal, and antigen specific B cells are grown. B cells secreting reactive antibodies for the γδ T cell antigens are identified by an antigen-binding ELISA screening of the B cell culture supernatants. High binding ELISA plates are coated with the γδ T cell antigen overnight. The ELISA plates are blocked, and diluted B cell culture supernatants are added to the plates. The plates are incubated at room temperature and following incubation, a secondary antibody specific for recognizing the yd T cell antigen antibody is added to the plate to determine if the γδ T cell antigen antibody bound the γδ T cell antigen. Binding of the antibody is determined by reaction of a substrate on the secondary antibody.

After the identification of monoclonal antibodies that are capable of binding γδ T cell antigens, the variable regions of the heavy and light chains of the yo T cell antibody are sequenced. Constructs are created for the expression of the heavy and light chain of the γδ T cell antibody. The constructs are transfected into a host cell to express the heavy and light chains, and the γδ T cell antibody is isolated from the supernatant.

### 1.2: Production of γδ T Cell Bispecific Antibodies

The variable region sequence of the γδ T cell monoclonal antibody and a second monoclonal antibody capable of binding a target antigen on a target cell of interest are used to generate a bispecific antibody to be tested for γδ T cell re-directed killing of the target cells. Target antigens of interest can be selected from, but not limited, antigens described in Zhang et al., Nucleic Acids Research 47(D1):D721-D728 (2019). γδ T cell bispecific antibodies are produced as full-length antibodies in the knob-into-hole format as human IgG4, as previously described (Atwell et al., J. Mol. Biol. 270:26-35 (1997)). Nucleic acid sequences encoding variable regions are sub-cloned into custom mammalian expression vectors containing the constant region of IgG4 expression cassettes using standard PCR restriction enzyme based cloning techniques. The bispecific antibodies are expressed by transient transfection in Chinese hamster ovary cell line. The antibodies are initially purified by MAB SELECT SURE Protein A column (GE Healthcare, Piscataway, New Jersey) (Brown, Bottomley et al. Biochem Soc Trans. 1998 August; 26 (3):S249). The column is equilibrated with Phosphate Buffer Saline (PBS), pH 7.2 and is loaded with fermentation supernatant at a flow rate of 2 mL/min. After loading, the column is washed with PBS (4 column volumes (CV)) followed by elution in 30 mM sodium acetate, pH 3.5. Fractions containing protein peaks as monitored by absorbance at 280 nm in Akta Explorer (GE healthcare) are pooled together and are neutralized to pH 5.0 by adding 1% of 3M sodium acetate, pH 9.0. As a polishing step, the antibodies are purified on a preparative size exclusion chromatography (SEC) using a SUPERDEX 200 column (GE healthcare). The integrity of sample is assessed by endotoxin measurement and SDS polyacrylamide gel electrophoresis under reducing and non-reducing conditions. The final protein concentrations are determined.

### 1.3: Production of Anti-TRDV2 Bispecific Antibodies

Variable region sequences of an anti-TRDV2 monoclonal antibody and a second monoclonal antibody capable of binding a target antigen on a target cell of interest are used to generate a bispecific antibody to be tested for γδ T cell re-directed killing of the target cells. Target antigens of interest can be selected from, but not limited to, antigens described in Zhang et al., Nucleic Acids Research 47(D1):D721-D728 (2019). Anti-TRDV2 bispecific antibodies are produced as full-length antibodies in the knob-into-hole format as human IgG1, as previously described (Atwell et al., J. Mol. Biol. 270:26-35 (1997)). Nucleic acid sequences encoding variable regions are sub-cloned into custom mammalian expression vectors containing the constant region of IgG1 expression cassettes using standard PCR restriction enzyme based cloning techniques. The bispecific antibodies are expressed by transient transfection in Chinese hamster ovary cell line. The antibodies are initially purified by Mab Select SuRe Protein A column (GE Healthcare, Piscataway, New Jersey) (Brown, Bottomley et al. Biochem Soc Trans. 1998 August; 26 (3):S249). The column is equilibrated with Phosphate Buffer Saline (PBS), pH 7.2 and is loaded with fermentation supernatant at a flow rate of 2 mL/min. After loading, the column is washed with PBS (4 column volumes (CV)) followed by elution in 30 mM sodium acetate, pH 3.5. Fractions containing protein peaks as monitored by Absorbance at 280 nm in Akta Explorer (GE healthcare) are pooled together and are neutralized to pH 5.0 by adding 1% of 3M sodium acetate, pH 9.0. As a polishing step, the antibodies are purified on a preparative size exclusion chromatography (SEC) using a SUPERDEX 200 column (GE healthcare). The integrity of sample is assessed by endotoxin measurement and SDS polyacrylamide gel electrophoresis under reducing and non-reducing conditions. The final protein concentrations are determined.

### Example 2—Bispecific Antibodies that Bind TRDV2 and a Cancer Antigen

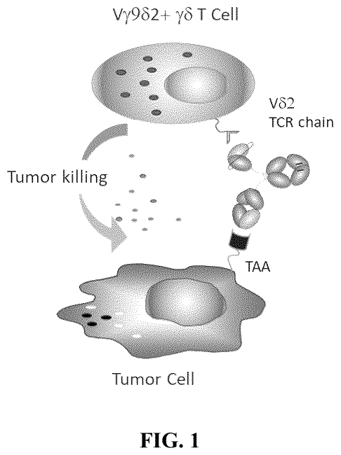

Examples 2.1-2.4 are based on the premise that γδ T cells, which mainly express heterodimers of TRDV2 and Vδ2 chains demonstrate potent anti-tumor functions. These cells express TCR-TRDV2 and the majority, if not all, of these cells exhibit efficient cytotoxicity of tumor target cells. This ability is then harnessed using bispecific antibodies constructed such that one arm binds to the TRDV2 structure and the other arm binds to a tumor-associated antigen expressed by the tumor cells. Thus, the bispecific antibody bridges the effector and target cells together-resulting in tumor cell killing. This mechanism of action is described in the schematic outlined in FIG. 1.

The subsequent examples can be divided into the following categories: (1) Generation and characterization of bispecific antibodies capable of binding to the TRDV2 arm expressed on γδ T cells and a cancer antigen (e.g., CD33) on cancer cells (Examples 2.1, 2.2, and 2.3); and (2) evidence for bispecific antibody-enabled target cell killing by γδ T cells expanded in vitro (Example 2.4).

### Example 2.1: Production of Anti-TRDV2 MAB

The mouse IgG1 anti-human T cell receptor TRDV2 clone B6 was sourced commercially. Sample preparation and LC/MSMS analysis were performed by Lake Pharma. (San Carlos, CA). The sample was reduced and alkylated, divided into seven aliquots, and proteolytically digested with Trypsin/LysC, Chymotrypsin, LysC, Pepsin, and AspN, Elastase, and Proteinase K enzymes. Resulting peptides were desalted using a ZIPTIP C18 Pipette Tips and separated on-line using reverse phase chromatography. Mass spectrometry was performed on Thermo Q-EXACTIVE spectrometer using HCD fragmentation. MS data sets were analyzed using PEAKS software by matching de novo sequence tags to an IMGT-based antibody sequences database. Gaps in the sequence were assigned using Contig sequence assembly of de novo identified peptides. All CDRs and hyper-mutations were confirmed by inspecting the MS/MS spectra.

The sequences obtained are shown in Tables 1 and 2.

TABLE 1
CDR Sequences of anti-TRDV2 mAb.

| | | SEQ | | SEQ | | SEQ |
|---|---|---|---|---|---|---|
| | | ID | | ID | | ID |
| Antibody | HCDR1 | NO: | HCDR2 | NO: | HCDR3 | NO: |
| B6 | ENPMH | 1 | IIYTDTGEPTYAAEFKG | 2 | EGGSHWYLDV | 3 |
| | | SEQ | | SEQ | | SEQ |
| | | ID | | ID | | ID |
| Antibody | LCDR1 | NO: | LCDR2 | NO: | LCDR3 | NO: |
| B6 | RASESVDKYGISFMN | 4 | AASNQGS | 5 | QQSKEVPRT | 6 |

TABLE 2
Heavy chain and light chain sequences of anti-TRDV2 mAb.

| | | SEQ |
|---|---|---|
| | | ID |
| mAb | Heavy Chain Amino Acid Sequence | NO: |
| B6 | QIQLVQSGPELKKPGETVKISCKASGYIFTENPMHWVKQAPGKGFKWMGIIY | 7 |
| | TDTGEPTYAAEFKGRFAFSLETSASTAYLQINYIKTEDTATYFCVREGGSHW | |
| | YLDVWGTGTTVTVSS | |
| | | SEQ |
| | | ID |
| | Light Chain Amino Acid Sequence | NO: |
| B6 | DIVLTQSPASLAVSLGQRATISCRASESVDKYGISFMNWFQQKPGQPPKLLIYA | 8 |
| | ASNQGSGVPARFSGSGSGTDFSLNIHPMEEDDTAMYFCQQSKEVPRTFGGGT | |
| | KLEIK | |

### Example 2.2: Preparation of Anti-TRDV2/Anti-CD33 Bispecific Antibodies

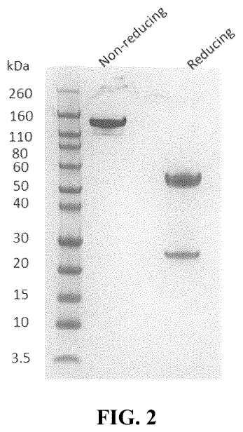

The variable region sequence of B6 (anti-TRDV2) and C33B904 (anti-CD33 antibody) (HCDRs and LCDRs in Table 3, HC and LC in Table 4) was used to generate a bispecific antibody to be tested for T cell re-directed killing of acute myeloid leukemia (AML) cells. The bispecific antibodies: VG56 (anti-TRDV2×CD33) and VG53 (anti-TRDV2×Null) were produced as full-length antibodies in the knob-into-hole format as human IgG1, as previously described (Atwell et al. J. Mol. Biol. 270:26-35, 1997). Nucleic acid sequences encoding variable regions were sub-cloned into a custom mammalian expression vectors containing constant region of IgG1 expression cassettes using standard PCR restriction enzyme based cloning techniques, and sequenced verified. The bispecific antibodies were expressed by transient transfection in Chinese hamster ovary cell line. The antibodies were initially purified by Mab Select SuRe Protein A column (GE Healthcare, Piscataway, New Jersey) (Brown, Bottomley et al. Biochem Soc Trans. 26 (3):S249, 1998). The column was equilibrated with Phosphate Buffer Saline (PBS), pH 7.2 and loaded with fermentation supernatant at a flow rate of 2 mL/min. After loading, the column was washed with PBS (4 column volumes (CV)) followed by elution in 30 mM sodium acetate, pH 3.5. Fractions containing protein peaks as monitored by Absorbance at 280 nm in Akta Explorer (GE healthcare) were pooled together and were neutralized to pH 5.0 by adding 1% of 3M sodium acetate, pH 9.0. As a polishing step, the antibodies were purified on a preparative size exclusion chromatography (SEC) using a SUPERDEX 200 column (GE healthcare). The integrity of sample was assessed by endotoxin measurement and SDS polyacrylamide gel electrophoresis under reducing and non-reducing conditions. A representative gel for VG56 is shown in FIG. 2. The final protein concentrations were 1.0 mg/ml for anti-TRDV2/anti-CD33 and 1.0 mg/mL for anti-TRDV2/Null. The final EU levels of anti-TRDV2/anti-CD33 and anti-TRDV2/Null based on these were <3.0 EU/mg.

TABLE 3
CDR Sequences of anti-CD33 mAb.

| | | SEQ | | SEQ | | SEQ |
|---|---|---|---|---|---|---|
| | | ID | | ID | | ID |
| Antibody | HCDR1 | NO: | HCDR2 | NO: | HCDR3 | NO: |
| C33B904 | DYAMH | 9 | GIGWSGGSIVYADSVKG | 10 | DSPYGDFFDY | 11 |
| | | SEQ | | SEQ | | SEQ |
| | | ID | | ID | | ID |
| Antibody | LCDR1 | NO: | LCDR2 | NO: | LCDR3 | NO: |
| C33B904 | KSSQTVFYSSNNKNYLA | 12 | WASTRKS | 13 | QHYYSTPYT | 14 |

TABLE 4
Heavy chain and light chain sequences of anti-CD33 mAb.

| | | SEQ |
|---|---|---|
| | | ID |
| mAb ID | Heavy Chain Amino Acid Sequence | NO: |
| C33B904 | EVQLVESGGGLVQPGRSLRLSCAASGFTFDDYAMHWVRQAPGKGLE | 15 |
| | WVSGIGWSGGSIVYADSVKGRFTISRDNAKNSLYLQMNSLRAEDTAL | |
| | YYCAKDSPYGDFFDYWGQGTLVTVSS | |
| | | SEQ |
| | | ID |
| | Light Chain Amino Acid Sequence | NO: |
| C33B904 | DIVMTQSPDSLAVSLGERATINCKSSQTVFYSSNNKNYLAWYQQKPGQPPKL | 16 |
| | LISWASTRKSGVPDRFSGSGSGTDFTLTVSSLQAEDVAVYYCQHYYSTPYTF | |
| | GQGTKLEIK | |

TABLE 5
Sequences of half antibodies expressed in CHO cells.

| | | SEQ |
|---|---|---|
| | | ID |
| mAb ID | ‘Knob’ arm and ‘hole’ arm amino acid sequence | NO: |
| ANTI- | MAWVWTLLFLMAAAQSIQADIVLTQSPASLAVSLGQRATISCRASESVDKY | 17 |
| TRDV2 | GISFMNWFQQKPGQPPKLLIYAASNQGSGVPARFSGSGSGTDFSLNIHPMEE | |
| (light | DDTAMYFCQQSKEVPRTFGGGTKLEIKRTVAAPSVFIFPPSDEQLKSGTASV | |
| chain) | VCLLNNFYPREAKVQWKVDNALQSGNSQESVTEQDSKDSTYSLSSTLTLSK | |
| | ADYEKHKVYACEVTHQGLSSPVTKSFNRGEC | |
| ANTI- | MAWVWTLLFLMAAAQSIQAQIQLVQSGPELKKPGETVKISCKASGYIFTENP | 18 |
| TRDV2 | MHWVKQAPGKGFKWMGIIYTDTGEPTYAAEFKGRFAFSLETSASTAYLQIN | |
| (‘hole’ | YIKTEDTATYFCVREGGSHWYLDVWGTGTTVTVSSASTKGPSVFPLAPSSKS | |
| arm) | TSGGTAALGCLVKDYFPEPVTVSWNSGALTSGVHTFPAVLQSSGLYSLSSV | |
| | VTVPSSSLGTQTYICNVNHKPSNTKVDKRVEPKSCDKTHTCPPCPAPEAAGG | |
| | PSVFLFPPKPKDTLMISRTPEVTCVVVDVSHEDPEVKFNWYVDGVEVHNAK | |
| | TKPREEQYNSTYRVVSVLTVLHQDWLNGKEYKCKVSNKALPAPIEKTISKA | |
| | KGQPREPQVYTLPPSREEMTKNQVSLSCAVKGFYPSDIAVEWESNGQPENN | |
| | YKTTPPVLDSDGSFFLVSKLTVDKSRWQQGNVFSCSVMHEALHNRFTQKSL | |
| | SLSPGK | |
| anti- | MAWVWTLLFLMAAAQSIQADIVMTQSPDSLAVSLGERATINCKSSQTVFYS | 19 |
| CD33 | SNNKNYLAWYQQKPGQPPKLLISWASTRKSGVPDRFSGSGSGTDFTLTVSSL | |
| scFv | QAEDVAVYYCQHYYSTPYTFGQGTKLEIKGGSEGKSSGSGSESKSTGGSEV | |
| (‘knob’ | QLVESGGGLVQPGRSLRLSCAASGFTFDDYAMHWVRQAPGKGLEWVSGIG | |
| arm) | WSGGSIVYADSVKGRFTISRDNAKNSLYLQMNSLRAEDTALYYCAKDSPYG | |
| | DFFDYWGQGTLVTVSSEPKSSDKTHTCPPCPAPEAAGGPSVFLFPPKPKDTL | |
| | MISRTPEVTCVVVDVSHEDPEVKFNWYVDGVEVHNAKTKPREEQYNSTYR | |
| | VVSVLTVLHQDWLNGKEYKCKVSNKALPAPIEKTISKAKGQPREPQVYTLP | |
| | PSREEMTKNQVSLWCLVKGFYPSDIAVEWESNGQPENNYKTTPPVLDSDGS | |
| | FFLYSKLTVDKSRWQQGNVFSCSVMHEALHNHYTQKSLSLSPGK | |
| Anti- | MAWVWTLLFLMAAAQSIQADIVMTQSPDSLAVSLGERATINCRASQSVDY | 20 |
| RSV | NGISYMHWYQQKPGQPPKLLIYAASNPESGVPDRFSGSGSGTDFTLTISSLQ | |
| scFv | AEDVAVYYCQQIIEDPWTFGQGTKVEIKGGSEGKSSGSGSESKSTGGSQITL | |
| (‘knob’ | KESGPTLVKPTQTLTLTCTFSGFSLSTSGMGVSWIRQPPGKALEWLAHIYWD | |
| arm) | DDKRYNPSLKSRLTITKDTSKNQVVLTMTNMDPVDTATYYCARLYGFTYG | |
| | FAYWGQGTLVTVSSEPKSSDKTHTCPPCPAPEAAGGPSVFLFPPKPKDTLMI | |
| | SRTPEVTCVVVDVSHEDPEVKFNWYVDGVEVHNAKTKPREEQYNSTYRVV | |
| | SVLTVLHQDWLNGKEYKCKVSNKALPAPIEKTISKAKGQPREPQVYTLPPSR | |
| | EEMTKNQVSLWCLVKGFYPSDIAVEWESNGQPENNYKTTPPVLDSDGSFFL | |
| | YSKLTVDKSRWQQGNVFSCSVMHEALHNHYTQKSLSLSPGK | |

### Example 2.3: Characterization of Vδ2+(γδ) T Cells and PAN T Cells

Zoledronic acid selectively expands Vδ2+γδ T cells from whole PBMCs. PBMCs were isolated from whole fresh PBMCs using EasySep™ Human γδ T cell isolation kit (Stem cell Technologies; Vancouver, CA) according to manufacturer instructions. Isolated PBMCs were cultured in RPMI-10 (RPMI supplemented with 10% FBS, 1× Pen/Strep) medium with recombinant human IL-2 (rhIL-2) to a final concentration of 1000 IU/mL and recombinant human IL-15 (rhIL-15) to a final concentration of 10 ng/mL and Zoledronic acid to a final concentration of 5 μM for 14 days.

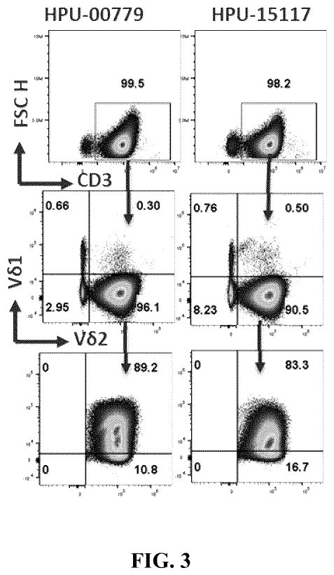

Numbers in representative dot plots show the frequency (mean±SEM) of Vδ2+ and Vδ2-TCR γδ T cells among total PBMCs cultured with Zoledronic acid+IL-2+IL-15 (right). Represented data is mean (±SEM) of five donors (n=5) from a single experiment (FIG. 3).

### Example 2.4: Evaluation of Binding and Cytotoxic Properties of the Anti-TRDV2/Anti-CD33 Bispecific Antibody Using Kasumi-3 Cells and Human γδ T Cells

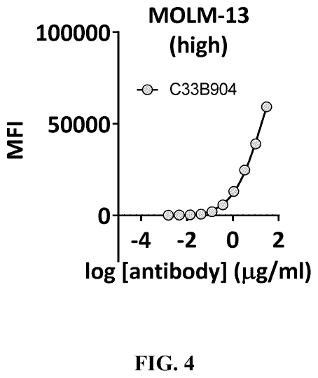

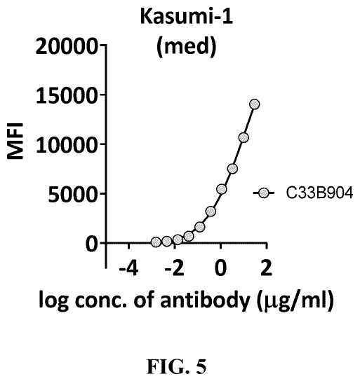

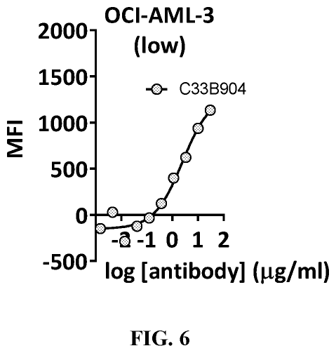

The binding of anti-CD33 clone C33B904 to a panel CD33+ cell lines were measured by FACS. The EC₅₀ and EC₉₀ were calculated for MOLM-13 (FIG. 4), Kasumi-1 (FIG. 5) and OCI-AML-3 (FIG. 6).

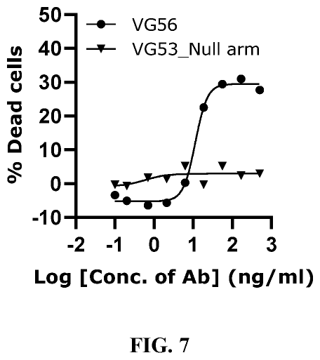

FIG. 7 shows that the anti-TRDV2/anti-CD33 bispecific antibody mediates γδ T cell cytotoxicity (from whole PBMCs) against CD33 expressing MOLM-13 cells in vitro. Healthy donor derived PBMCs (Effectors), cultured with Zoledronic acid+IL-2+IL-15 for 12 days, were co-cultured with CFSE labelled MOLM-13 cells (Targets) at 1:1 E:T ratios in the presence of various concentrations of the bispecific antibody for 24 hours. Dose response curves show anti-TRDV2/anti-CD33 and anti-TRDV2/anti-NULL bispecific mediated γδ T cell cytotoxicity against CD33 expressing kasumi-3 cells in a dose dependent manner (FIG. 7). Cytotoxicity values represented here were subtracted of basal cytotoxicity value observed in the absence of bispecific antibody. EC₅₀ values were calculated as described in methods. Representative data shown here are from a single experiment.

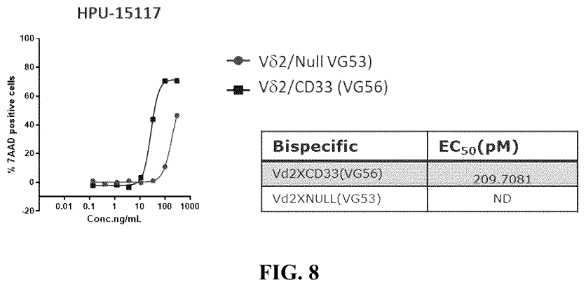

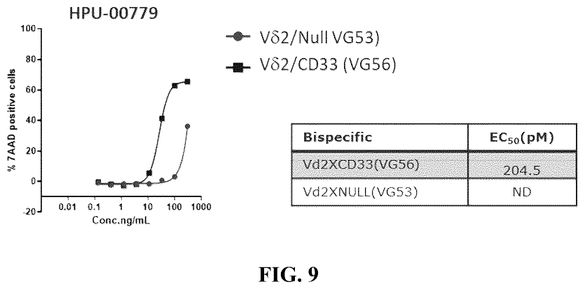

FIGS. 8-9 show that the anti-TRDV2/anti-CD33 bispecific antibody mediates γδ T cell cytotoxicity against CD33 expressing Kasumi-3 cells in vitro. Enriched γδ T cells (Effectors), isolated from PBMCs cultured with Zoledronic acid+IL-2+IL-15 for 14 days, were co-cultured with CFSE labelled Kasumi-3 cells (Targets) at 1:1 E:T ratios in the presence of various concentrations of the bispecific antibody for 24 hours. Dose response curves show anti-TRDV2/anti-CD33 and anti-TRDV2/anti-NULL bispecific mediated γδ T cell cytotoxicity against CD33 expressing kasumi-3 cells in a dose dependent manner at 1:1 (FIGS. 8-9) E:T ratios. Cytotoxicity values represented here were subtracted of basal cytotoxicity value observed in the absence of bispecific antibody. EC₅₀ values were calculated as described in methods. Representative data shown here are from a single experiment.

### Example 3-Bispecific Antibodies that Bind TRDV2 and a Cancer Cell Antigen

### Example 3.1: Preparation of Bispecific Antibodies that Bind TRDV2 and a Cancer Cell Antigen

Variable region sequences of an anti-TRDV2 monoclonal antibody and a second monoclonal antibody capable of binding an antigen on a T cell of interest are used to generate a bispecific antibody to be tested for γδ T cell re-directed killing of the target T cells.

Exemplary TRDV2 VH CDR1, VH CDR2, VH CDR3, VL CDR1, VL CDR2 and VL CDR3 sequences are provided in Table 1; and exemplary VH domain and VL domain sequences are provided in Table 2. However, any TRDV2 antibodies may be used for the preparation of the bispecific antibodies.

The second monoclonal antibody that binds a T cell antigen, is an antibody that binds a cancer antigen, as provided elsewhere herein. In addition to CD33, exemplary cancer antigens include, but are not limited to: angiopoietin, BCMA, CD19, CD20, CD22, CD25 (IL2-R), CD30, CD37, CD38, CD52, CD56, CD123 (IL-3R), cMET, DLL/Notch, EGFR, EpCAM, FGF, FGF-R, GD2, HER2, Mesothelin, Nectin-4, PDGFRα, RANKL, SLAMF7, TROP2, VEGF, or VEGF-R. In some embodiments, the cancer antigen is CEA, immature laminin receptor, TAG-72, HPV E6, HPV E7, BING-4, calcium-activated chloride channel 2, cyclin-B1, 9D7, EpCAM, EphA3, Her2/neu, telomerase, mesothelin, SAP-1, surviving, a BAGE family antigen, CAGE family antigen, GAGE family antigen, MAGE family antigen, SAGE family antigen, XAGE family antigen, NY-ESO-1/LAGE-1, PRAME, SSX-2, Melan-A, MART-1, Gp100, pmel17, tyrosinase, TRP-1, TRP-2, P. polypeptide, MCIR, prostate-specific antigen, β-catenin, BRCA1, BRCA2, CDK4, CML66, fibronectin, MART-2, p53, Ras, TGF-βRII, or MUC1.

Anti-TRDV2 bispecific antibodies are produced as full-length antibodies in the knob-into-hole format as human IgG1, as previously described (Atwell et al., J. Mol. Biol. 270:26-35 (1997)).

Nucleic acid sequences encoding variable regions are sub-cloned into custom mammalian expression vectors containing the constant region of IgG1 expression cassettes using standard PCR restriction enzyme based cloning techniques.

The bispecific antibodies are expressed by transient transfection in a CHO cell line. The antibodies are initially purified by MAB SELECT SURE Protein A column (GE Healthcare, Piscataway, New Jersey) (Brown, Bottomley et al. Biochem Soc Trans. 1998 August; 26 (3): S249). The column is equilibrated with PBS, pH 7.2 and is loaded with fermentation supernatant at a flow rate of 2 mL/min. After loading, the column is washed with PBS (4 column volumes (CV)) followed by elution in 30 mM sodium acetate, pH 3.5. Fractions containing protein peaks as monitored by absorbance at 280 nm in Akta Explorer (GE healthcare) are pooled together and are neutralized to pH 5.0 by adding 1% of 3M sodium acetate, pH 9.0. As a polishing step, the antibodies are purified on a preparative size exclusion chromatography (SEC) using a SUPERDEX 200 column (GE healthcare). The integrity of sample is assessed by endotoxin measurement and SDS polyacrylamide gel electrophoresis under reducing and non-reducing conditions. The final protein concentrations are determined.

### Example 3.2: Evaluation and Binding of Bispecific Antibodies that Bind TRDV2 and a Cancer Antigen

Assessment of binding of the bispecific antibody to Vδ2+γδ T cells and target cells expressing the T cell antigen, and resulting cytotoxicity, will be determined in vitro.

Enriched γδ T cells (effectors), isolated from PBMCs cultured with zoledronic acid, IL-2, and IL-15 for 12 days, are co-cultured with CFSE-labelled cells expressing the T cell antigen (targets) at 1:1, 5:1 and 10:1 E:T ratios in the presence of various concentrations of the bispecific antibody for 24 hours. An anti-TRDV2/anti-NULL bispecific antibody will be used as a control. Cytotoxicity values are determined by subtracting basal cytotoxicity values observed in the absence of bispecific antibodies. Dose response curves are calculated to determine if bispecific mediated γδ T cell cytotoxicity occurs against the target cells expressing the T cell antigen in a dose dependent manner at 1:1, 5:1, and 10:1 E:T ratios.

In addition, selective activation of Vδ2+γδ T cells is assessed by co-culturing whole fresh PBMCs with target cells expressing the T cell antigen in the presence of various concentrations of the anti-TRDV2/anti-T cell antigen bispecific antibody for 72 hours at 37° C. As a positive and negative control, co-cultured cells were stimulated with anti-CD3/anti-T cell antigen and anti-TRDV2/anti-NULL bispecifics for 72 hours at 37° C. The frequency of Vδ2+, Vδ2-γδ T cells and non-yo T cells positive for CD69, CD25 surface expression, and intracellular Granzyme B expression is determined.

It will be appreciated by those skilled in the art that changes could be made to the embodiments described above without departing from the broad inventive concept thereof. It is understood, therefore, that this invention is not limited to the particular embodiments disclosed, but it is intended to cover modifications within the spirit and scope of the present invention as defined by the present description.

1. A multispecific antibody comprising:
(a) a first binding domain that binds to T Cell Receptor Delta Variable 2 (TRDV2), and
(b) a second binding domain that binds to an antigen on the surface of a cancer cell, wherein the first binding domain comprises:
a heavy chain variable region (VH) comprising a VH complementarity determining region (CDR) 1 having the amino acid sequence of SEQ ID NO:1, a VH CDR2 having the amino acid sequence of SEQ ID NO:2, and a VH CDR3 having the amino acid sequence of SEQ ID NO:3, and
a light chain variable region (VL) comprising a VL CDR1 having an amino acid sequence of SEQ ID NO:4, a VL CDR2 having an amino acid sequence of SEQ ID NO:5, and a VL CDR3 having an amino acid sequence of SEQ ID NO:6.

2. The multispecific antibody of claim 1, wherein the first binding domain comprises:
(i) a VH having an amino acid sequence of SEQ ID NO:7; and
(ii) a VL having an amino acid sequence of SEQ ID NO:8.

3. (canceled)

4. (canceled)

5. The multispecific antibody of claim 1, wherein
(i) the first binding domain comprises a VH having an amino acid sequence of SEQ ID NO: 7;
(ii) the first binding domain comprises a VL having an amino acid sequence of SEQ ID NO: 8; or
(iii) the first binding domain comprises a VH having an amino acid sequence of SEQ ID NO: 7, and a VL having an amino acid sequence of SEQ ID NO:8.

6. The multispecific antibody of claim 1, wherein the VH CDR1, VH CDR2, VH CDR3, VL CDR1, VL CDR2 and VL CDR3 of the first binding domain form a binding site for an antigen of the TRDV2 or an epitope of the TRDV2.

7. The multispecific antibody of claim 1, wherein the TRDV2 is present on the surface of a T cell.

8. The multispecific antibody of claim 1, wherein the cancer cell is a cell of an adrenal cancer, anal cancer, appendix cancer, bile duct cancer, bladder cancer, bone cancer, brain cancer, breast cancer, cervical cancer, colorectal cancer, esophageal cancer, gallbladder cancer, gestational trophoblastic, head and neck cancer, Hodgkin lymphoma, intestinal cancer, kidney cancer, leukemia, liver cancer, lung cancer, melanoma, mesothelioma, multiple myeloma, neuroendocrine tumor, non-Hodgkin lymphoma, oral cancer, ovarian cancer, pancreatic cancer, prostate cancer, sinus cancer, skin cancer, soft tissue sarcoma spinal cancer, stomach cancer, testicular cancer, throat cancer, thyroid cancer, uterine cancer endometrial cancer, vaginal cancer, or vulvar cancer.

9. The multispecific antibody of claim 1, wherein the antigen on the surface of the cancer cell is angiopoietin, BCMA, CD19, CD20, CD22, CD25 (IL2-R), CD30, CD33, CD37, CD38, CD52, CD56, CD123 (IL-3R), cMET, DLL/Notch, EGFR, EpCAM, FGF, FGF-R, GD2, HER2, Mesothelin, Nectin-4, PAP, PDGFRα, PSA, PSA3, PSMA, RANKL, SLAMF7, STEAP1, TARP, TROP2, VEGF, VEGF-R, CEA, immature laminin receptor, TAG-72, HPV E6, HPV E7, BING-4, calcium-activated chloride channel 2, cyclin-B1, 9D7, EpCAM, EphA3, Her2/neu, telomerase, mesothelin, SAP-1, surviving, a BAGE family antigen, CAGE family antigen, GAGE family antigen, MAGE family antigen, SAGE family antigen, XAGE family antigen, NY-ESO-1/LAGE-1, PRAME, SSX-2, Melan-A, MART-1, Gp100, pmel17, tyrosinase, TRP-1, TRP-2, P. polypeptide, MC1R, prostate-specific antigen, β-catenin, or BRCA1.

10. The multispecific antibody of claim 1, wherein the antigen on the surface of the cancer cell is CD33,
wherein optionally the second binding domain that binds CD33 comprises:
(i) a VH comprising a VH CDR1, a VH CDR2, and a VH CDR3 having an amino acid sequence of a VH CDR1, a VH CDR2, and a VH CDR3, respectively, of a VH having the amino acid sequence of SEQ ID NO: 15; and
(ii) a VL comprising a VL CDR1, a VL CDR2, and a VL CDR3 having an amino acid sequence of a VL CDR1, a VL CDR2, and a VL CDR3, respectively, of a VL having the amino acid sequence of SEQ ID NO: 16; and
wherein optionally
(i) the VH CDR1, VH CDR2, VH CDR3, VL CDR1, VL CDR2, and VL CDR3 amino acid sequences of the second binding domain are according to the Kabat numbering system;
(ii) the VH CDR1, VH CDR2, VH CDR3, VL CDR1, VL CDR2, and VL CDR3 amino acid sequences of the second binding domain are according to the Chothia numbering system;
(iii) the VH CDR1, VH CDR2, VH CDR3, VL CDR1, VL CDR2, and VL CDR3 amino acid sequences of the second binding domain are according to the AbM numbering system;
(iv) the VH CDR1, VH CDR2, VH CDR3, VL CDR1, VL CDR2, and VL CDR3 amino acid sequences of the second binding domain are according to the Contact numbering system;
(v) the VH CDR1, VH CDR2, VH CDR3, VL CDR1, VL CDR2, and VL CDR3 amino acid sequences of the second binding domain are according to the IMGT numbering system; or
(vi) the VH CDR1, VH CDR2, VH CDR3, VL CDR1, VL CDR2, and VL CDR3 amino acid sequences of the second binding domain are according to the Exemplary numbering system.

11. (canceled)

12. (canceled)

13. The multispecific antibody of claim 10, wherein the second binding domain comprises:
(i) a VH comprising a VH CDR1 having an amino acid sequence of SEQ ID NO:9, a VH CDR2 having an amino acid sequence of SEQ ID NO:10, and a VH CDR3 having an amino acid sequence of SEQ ID NO:11; and
(ii) a VL comprising a VL CDR1 having an amino acid sequence of SEQ ID NO:12, a VL CDR2 having an amino acid sequence of SEQ ID NO:13, and a VL CDR3 having an amino acid sequence of SEQ ID NO: 14, and
wherein optionally the second binding domain comprises a VH having the amino acid sequence of SEQ ID NO:15, and/or a VL having the amino acid sequence of SEQ ID NO:16.

14. (canceled)

15. (canceled)

16. (canceled)

17. The multispecific antibody of claim 1, wherein the multispecific antibody is a bispecific antibody, a trispecific antibody, or a quadraspecific antibody.

18. The multispecific antibody of claim 1, wherein the antibody
(i) is a humanized antibody,
(ii) is an IgG antibody, optionally wherein the IgG antibody is an IgG1, IgG2, IgG3, or IgG4 antibody,
(iii) comprises a kappa light chain or a lambda light chain, or
(iv) is a monoclonal antibody.

19. A multispecific antibody comprising: a first means capable of binding TRDV2 on the surface of a γδ T cell; and a second means capable of binding a cancer antigen.

20. A nucleic acid encoding the multispecific antibody of claim 1.

21. A vector comprising the nucleic acid of claim 20.

22. A host cell comprising the vector of claim 21.

23. A kit comprising the vector of claim 21 and packaging for the same.

24. A pharmaceutical composition comprising the multispecific antibody of claim 1, and a pharmaceutically acceptable carrier.

25. A process for making an antibody that binds to more than one target molecule, the molecule comprising: a step for performing a function of obtaining a binding domain capable of binding to TRDV2 antigen on a γδ T cell; a step for performing a function of obtaining a binding domain capable of binding to an antigen on the surface of a cancer cell; and a step for performing a function of providing an antibody capable of binding to a TRDV2 antigen on a γδ T cell and an antigen on the surface of a cancer cell, wherein optionally wherein the step for performing the function of obtaining the binding domain capable of binding to the antigen on the surface of a cancer cell is repeated n times and further comprising n steps for performing a function of providing the binding domain capable of binding to a TRDV2 antigen on a γδ T cell and n number of target molecules, wherein n is at least 2.

26. (canceled)

27. A method of directing a γδ T cell expressing TRDV2 to a cancer cell, the method comprising contacting the γδ T cell with the multispecific antibody of claim 1, wherein the contacting directs the γδ T cell to the cancer cell.

28. A method of inhibiting growth or proliferation of cancer cells expressing a cancer antigen on the cell surface, the method comprising contacting the cancer cells with the multispecific antibody of claim 1, wherein contacting the cancer cells with the pharmaceutical composition inhibits growth or proliferation of the cancer cells, wherein optionally the cancer cells are in the presence of a γδ T cell expressing TRDV2 while in contact with the multispecific antibody.

29. (canceled)

30. A method for eliminating cancer cells in a subject, comprising administering an effective amount of the multispecific antibody of claim 1 to the subject, wherein optionally the subject is a subject in need thereof.

31. A method for treating cancer in a subject, comprising administering an effective amount of the multispecific antibody of claim 1 to the subject, wherein optionally the subject is a subject in need thereof.

32. (canceled)

33. A method of activating a yd T cell expressing TRDV2, comprising contacting the γδ T cell with the multispecific antibody of claim 1.
